(19) United States
(12) Patent Application Publication
(10) Pub. No.: US 2015/0031045 A1
(43) Pub. Date: Jan. 29, 2015

(54) MARKERS FOR DIAGNOSING AMYOTROPHIC LATERAL SCLEROSIS

(71) Applicant: University Of Maryland, College Park, MD (US)

(72) Inventors: Eva Chin, Silver Spring, MD (US); Dapeng Chen, Lanham, MD (US)

(21) Appl. No.: 14/511,757

(22) Filed: Oct. 10, 2014

Related U.S. Application Data
(63) Continuation-in-part of application No. PCT/US2013/036285, filed on Apr. 12, 2013.
(60) Provisional application No. 61/785,604, filed on Mar. 14, 2013.
(60) Provisional application No. 61/754,423, filed on Jan. 18, 2013.
(60) Provisional application No. 61/637,052, filed on Apr. 23, 2012.
(60) Provisional application No. 61/623,311, filed on Apr. 12, 2012.

(51) Int. Cl. G01N 33/53; C12Q 1/68

(52) U.S. Cl. CPC G01N 33/5308; C12Q 1/6883; G01N 2800/28; C12Q 2600/112
USPC 435/6.12; 435/7.9; 436/501

(57) ABSTRACT

Disclosed herein are markers for diagnosing amyotrophic lateral sclerosis (ALS) and for monitoring efficacy of treatment for ALS. These markers allow for early detection of ALS, namely before the onset of clinical symptoms. Also disclosed herein are methods for diagnosing ALS in a subject in need thereof, for monitoring the efficacy of a treatment for ALS in the subject, and for treatment of ALS the subject.

### CROSS-REFERENCE TO RELATED APPLICATIONS

This is a continuation-in-part of International Patent Application No. PCT/US2013/036285, filed on Apr. 12, 2013, which claims priority to U.S. Patent Application No. 61/785,604, filed on Mar. 14, 2013, U.S. Patent Application No. 61/754,423, filed on Jan. 18, 2013, U.S. Patent Application No. 61/637,052, filed on Apr. 23, 2012, and U.S. Patent Application No. 61/623,311, filed on Apr. 12, 2012, the entire contents of all of which are fully incorporated herein by reference.

### TECHNICAL FIELD

The present invention relates to markers for diagnosing amyotrophic lateral sclerosis (ALS), to markers for monitoring the efficacy of a treatment for ALS, to methods of diagnosing ALS, to methods for monitoring the efficacy of a treatment for ALS, and to methods for treatment of ALS.

### BACKGROUND

Amyotrophic Lateral Sclerosis (ALS or Lou Gehrig's Disease) is a progressive neurodegenerative disease characterized by muscle weakness, spasticity, and paralysis originating from selective motor neuron cell death. ALS is invariably fatal due to respiratory muscle failure, usually within 2-5 years of clinical symptom onset. The early symptoms (e.g., muscle weakness, muscle cramps, and abnormal fatigue of the arms and/or legs) of ALS are non-specific, and therefore, make early detection of ALS difficult. Additionally, current therapy for ALS (e.g., Riluzole) only extends survival by months.

ALS cases are classified as either sporadic (i.e., no known underlying familial or genetic component) or familial. Familial ALS results from inheritance of an allele of Cu,Zn-superoxide dismutase 1 (SOD1) gene, in which codon 93 is changed from a glycine residue to an alanine residue. SOD1 is a metalloprotein that prevents free radical-mediated oxidative damage to cells by catalyzing the dismutation of superoxide (O₂.⁻) to hydrogen peroxide (H₂O₂). The G93A substitution in SOD1 is a gain of function mutation, resulting in higher SOD1 activity and enhanced free-radical generating capacity. Such a gain of function mutation in mice (i.e., the G93A*SOD1 mouse) recapitulates the pathology of both sporadic and familial ALS.

Accordingly, a need exists for the identification and development of markers for the detection of ALS, especially early detection, to facilitate clinical treatment and management of disease progression. Furthermore, more effective treatments are required to delay disease progression and/or decrease mortality in subjects suffering from ALS.

### SUMMARY

The present invention is directed to a method for diagnosing amyotrophic lateral sclerosis (ALS) in a subject in need thereof, the method comprising: (a) obtaining a sample from the subject; (b) measuring levels of sarcoplasmic reticulum endoplasmic reticulum 1 (SERCA 1) and SERCA 2 proteins in the sample; and (c) comparing the levels measured in step (b) with levels of SERCA 1 and SERCA 2 proteins in a control, wherein a decrease in the levels of SERCA 1 and SERCA 2 proteins as compared to the control indicate that the subject is suffering from ALS. The sample may include at least one of a plasma sample, a serum sample, and a skeletal muscle tissue sample. The method may further comprise measuring a Ca²⁺ level in the sample, and comparing the Ca²⁺ level to a Ca²⁺ level in the control, wherein an increase in the Ca²⁺ level as compared to the control further indicates that the subject is suffering from ALS. The Ca²⁺ level may be an intracellular Ca²⁺ concentration.

The present invention is also directed to a method for diagnosing amyotrophic lateral sclerosis (ALS) in a subject in need thereof, the method comprising: (a) obtaining a sample from a subject; (b) measuring a level of endoplasmic reticulum (ER) chaperone immunoglobin binding protein (BiP) in the sample; and (c) comparing the level measured in step (b) with a level of BiP protein in a control, wherein an increase in the level of BiP protein as compared to the control indicates that the subject is suffering from ALS. The sample may include at least one of a plasma sample, a serum sample, and a skeletal muscle sample. The method may further comprise measuring a level of a protein selected from a group consisting of PERK, IRE1α, and PDI, and comparing the measured level of the protein to a level of a corresponding protein in the control, wherein an increase in the level of PERK, IRE1α, or PDI protein further indicates that the subject is suffering from ALS.

The present invention is further directed to a kit for early diagnosis of amyotrophic lateral sclerosis (ALS) in a subject, the kit comprising agents that bind and identify SERCA 1, SERCA 2, BiP, or a combination thereof. The agents may include antibodies. The kit may further comprise agents that detect a change in an mRNA selected from a group consisting of SERCA 2 mRNA, TnIs mRNA, Myoglobin mRNA, TnIf mRNA, GAPDH mRNA, MCK mRNA, and any combination thereof. The kit may further comprise agents that detect a change in an intracellular Ca²⁺ concentration. The kit may further comprise agents that bind and identify PERK, IRE1α, PDI, CHOP, Caspase-12, β-actin, α-tubulin, or a combination thereof.

The present invention is directed to a method for monitoring the efficacy of a treatment for amyotrophic lateral sclerosis (ALS) in a subject, the method comprising: (a) obtaining a first sample from the subject before the treatment and a second sample from the subject during or after treatment; (b) measuring a first level of a protein in the first sample and a second level of the protein in the second sample, wherein (i) the protein is selected from the group consisting of SERCA1 and PV; or (ii) the protein is selected from the group consisting of CHOP, Caspase-12, PERK, BiP, IRE1α, and PDI; and (c) comparing the first level of the protein and the second level of the protein, wherein (i) a second level of the protein during or after treatment of (b)(i) is higher than the first level of the protein of (b)(i) before treatment and is indicative of a therapeutic effect of the treatment in the subject; or (ii) a second level of the protein during or after treatment of (b)(ii) is lower than the first level of the protein of (b)(ii) before treatment and is indicative of a therapeutic effect of the treatment in the subject. The protein of (b)(i) may be SERCA1. The protein of (b)(ii) may be CHOP, PERK, BiP, IRE1α, or PDI.

The present invention is also directed to a method for treatment of amyotrophic lateral sclerosis (ALS) in a subject in need thereof, the method comprising administering a composition comprising a therapeutically effective amount of an agent, wherein the agent is 6-gingerol.

The present invention is further directed to a method for treatment of amyotrophic lateral sclerosis (ALS) in a subject in need thereof, the method comprising administering a composition comprising a therapeutically effective amount of an agent that increases a level of SERCA1 protein. The agent may decrease a level of CHOP protein, PERK protein, BiP protein, IRE1α protein, PDI protein, or any combination thereof.

### BRIEF DESCRIPTION OF THE DRAWINGS

FIG. 1 shows a schematic representation of alterations in excitation-contraction and excitation-transcription coupling in G93A*SOD1 ALS mice.

FIG. 2 shows intracellular Ca²⁺ transients in single muscle fibres from SOD1*G93A transgenic and control mice. Representative raw data traces for intracellular free Ca²⁺ concentration ([Ca²⁺]i) in single muscle fibres from control (CON; black line) and SOD1*G93A transgenic (ALS; gray line) mice at 70 (A), 91 (B) and 134 d (C). Intracellular Ca²⁺ transients are shown in response to 350 msec tetani at 10, 30, 50, 70 and 100 Hz. Note the increased resting/baseline [Ca²⁺]i at 91 d and 134 d as well as the elevated peak [Ca²⁺]i at 10 Hz and elevated Ca²⁺ tails at all stimulation frequencies.

FIG. 3 shows intracellular free Ca²⁺ concentration in response to electrical stimulation in SOD1*G93A transgenic and control mice. Average changes in (A) steady state tetanic [Ca²⁺]i for CON (♦; solid line) and ALS (▪; hatched line) mice at 120-140 d and in (B) resting [Ca²⁺]i for CON (solid bars) and ALS mice (hatched bars) at 70, 90 and 120-140 d. Data represent mean±SE for all fibres: 70 d (n=28), 90 d (n=30) and 120-140 d (n=42); * p<0.05. C) Raw data traces from representative fibres for 130 d old CON (black line) and ALS (blue line and red line) fibres showing the higher [Ca²⁺]i and slower decline in intracellular Ca²⁺ concentration following a 10 Hz Ca²⁺ transient. This expanded timescale shows the higher average steady state [Ca²⁺]i in ALS (“b” and “c”) relative to CON (“a”) fibres in response to 10 Hz stimulation.

FIG. 4 shows Maximum calcineurin activity in quadriceps muscle of control and G93A*SOD1 mice. Quadriceps muscle from 120-140 d old control (CON) and G93A*SOD1 1 (ALS) mice were homogenized and analyzed for calcineurin (CnA) activity.

FIG. 5 shows total and phospho NFATc1 in skeletal muscle of G93A*SOD1 ALS mice. Cytoplasmic protein was isolated from 120-140 d old wild-type (CON) and transgenic G93A*SOD1 (ALS) superficial gastrocnemius (SP-GAS) and western blot was performed by using antibody specific for NFATc1. NFATc1 bands range from 85-142 kD on Western blots with the lower molecular weight species of ˜85 kD, correspond to the hypo-phosphorylated form of NFATc1 (NFATc1) and the >90 kD forms representing the phosphorylated NFATc1 (pNFATc1). There is a higher abundance of both NFATc1 and pNFATc1 in CON vs. ALS samples. Upon activation, pNFATc1 translocates to the nucleus and disappears from the cytoplasm. Thus, in these cytoplasmic protein preparations, the less abundant NFATc1 protein (as NFATc1 or pNFATc1) in ALS muscle is interpreted as to their loss from the cytosol and translocation to nucleus.

FIG. 6 shows changes in slow fibre type-specific and oxidative gene expression markers in tibialis anterior muscle from SOD1*G93A transgenic and control mice. A) Troponin I slow (TnIs) and B) Myoglobin gene expression was assessed in tibialis anterior muscle from CON and ALS mice at 70 d (n=3), 90 d (n=3) and 120-140 d (n=5). TnIs and Myoglobin expression were assessed used qPCR for the target gene multiplexed with 18S and normalized gene expression calculated using the ΔCt method. Fold-changes in gene expression were then determined relative to CON1 (70 d) using the 2ΔΔCt method. Data shown are mean±SE. * p<0.05 vs. CON; ** p<0.01 vs. CON.

FIG. 7 shows changes in fast fibre type-specific and glycolytic gene expression markers in tibialis anterior muscle from SOD1*G93A transgenic and control mice. A) Troponin I fast (TnIf), B) Muscle Creatine Kinase (MCK) and C) glyceraldehyde phosphate dehydrogenase (GAPDH) gene expression was assessed in tibialis anterior muscle from CON and ALS mice at 70 d (n=3), 90 d (n=3) and 120-140 d (n=5). TnIf, MCK and GAPDH expression were assessed used qPCR for the target gene multiplexed with 18S and normalized gene expression calculated using the ΔCt method. Fold-changes in gene expression were then determined relative to CON1 (70 d) using the 2ΔΔCt method. Data shown are mean±SE. * p<0.05 vs. CON; ** p<0.01 vs. CON.

FIG. 8 shows protein levels for SERCA1 in superficial and deep portions of the gastrocnemius muscle of SOD1*G93A transgenic and control mice. Protein levels for SERCA1 were determined by western blot analysis and quantified by chemiluminescence. A) Raw data for superficial gastrocnemius muscle muscles for representative sample at 70 d, 90 d and 120-140 d. B) Average luminescence values obtained by densitometric analysis and expressed as arbitrary units are shown for superficial gastrocnemius muscle. Data shown represent mean±SE for CON and ALS mice at 70 d (n=3), 90 d (n=5) and 120-140 d (n=5); * p<0.05 vs. CON.

FIG. 9 shows protein levels for SERCA1 in deep portions of the gastrocnemius (DP-GAS) muscle of SOD1*G93A transgenic and control mice. Protein levels for SERCA1 were determined by western blot analysis and quantified by chemiluminescence. A) Raw data for superficial gastrocnemius muscle muscles for representative samples at 70 d, 90 d and 120-140 d. B) Average luminescence values obtained by densitometric analysis and expressed as arbitrary units are shown for superficial gastrocnemius muscle. Data shown represent mean±SE for CON and ALS mice at 70 d (n=3), 90 d (n=5) and 120-140 d (n=5); * p<0.05 vs. CON.

FIG. 10 shows protein and mRNA levels for SERCA2 in superficial gastrocnemius muscle of SOD1*G93A transgenic and control mice. A) SERCA2 protein level for superficial gastrocnemius muscle for representative sample at 70 d, 90 d and 120-140 d. Protein levels for SERCA2 were determined by western blot analysis and quantified by chemiluminescence. B) SERCA2 protein levels quantified by densitometric analysis and expressed as arbitrary units are shown for superficial gastrocnemius muscle. Data shown represent mean±SE for CON and ALS mice at 70 d (n=3), 90 d (n=5) and 120-140 d (n=5); * p<0.05 vs. CON. C) SERCA2 mRNA levels in tibialis anterior muscle for 2-3 representative samples at 70 d, 90 d and 120-140 d.

FIG. 11 shows protein levels for parvalbumin in superficial and deep gastrocnemius muscle of SOD1*G93A transgenic and control mice. Protein levels for PV were determined by western blot analysis and quantified by chemiluminescence. A) Raw data shown for superficial gastrocnemius muscle for representative sample at 70 d, 90 d and 120-140 d. Average luminescence values obtained by densitometric analysis and expressed as arbitrary units (AU) are shown for superficial (B) and deep (C) gastrocnemius muscle. Data shown represent mean±SE for CON and ALS mice at 70 d (n=3), 90 d (n=5) and 120-140 d (n=5); * p<0.05 vs. CON.

FIG. 12 shows protein levels for dihydropyridine receptor alpha 1 sub-unit in superficial gastrocnemius muscle of SOD1*G93A transgenic and control mice. Protein levels for DHPRα1 were determined by western blot analysis and quantified by chemiluminescence. A) Raw data shown for superficial gastrocnemius for 3 representative samples at 120-140 d. Membrane staining for total protein is shown to confirm equal loading. B) Average luminescence values obtained by densitometric analysis and expressed as arbitrary units (AU) are shown for superficial gastrocnemius muscle. Data shown represent mean±SE for CON and ALS mice at 120-140 d (n=5); * p<0.05 vs. CON.

FIG. 13 shows PERK and phospho-PERK are up-regulated in skeletal muscle of G93A*SOD1 ALS mice. (A) Protein was isolated from different ages of wild-type (CON) and transgenic G93A*SOD1 (ALS) superficial gastrocnemius and western blots were performed by using antibodies specific for PERK, phosphor-PERK, β-actin. Total protein was used as the loading control. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. (B) Analysis of average arbitrary units (AU) for PERK. (C) Analysis of average ratio of phosphor-PERK to total PERK. Data in B and C are presented as mean±S.E; *, p<0.05; **, p<0.01 CON versus ALS.

FIG. 14 shows IRE1α is up-regulated in skeletal muscle of G93A*SOD1 ALS mice. (A) Protein was isolated from different ages of wild-type (CON) and transgenic G93A*SOD1 (ALS) superficial gastrocnemius and western blot was performed by using antibody specific for IRE1α. Total protein was used as the loading control. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. (B) Analysis of average arbitrary units (AU) for IRE1α. Data in B are presented as mean±S.E; *, p<0.05; **, p<0.01 CON versus ALS.

FIG. 15 shows ER chaperone PDI is up-regulated in skeletal muscle of symptomatic G93A*SOD1 ALS mice. (A) Protein was isolated from different ages of wild-type (CON) and transgenic G93A*SOD1 (ALS) superficial gastrocnemius and western blot was performed by using antibody specific for PDI. Total protein was used as the loading control. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. (B) Analysis of average arbitrary units (AU) of PDI. Data in B are presented as mean±S.E; **, p<0.01 CON versus ALS.

FIG. 16 shows CHOP is up-regulated in skeletal muscle but not cardiac muscle of G93A*SOD1 ALS mice. (A) Protein was isolated from different ages of wild-type (CON) and transgenic G93A*SOD1 (ALS) superficial gastrocnemius (SP-GAS) and western blot was performed by using antibody specific for CHOP. Total protein was used for loading control. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. (B) Analysis of average arbitrary units (AU) of CHOP in SP-GAS. (C) Same as A, protein isolated from CON and ALS mice diaphragm muscle (DIA) and western blot was performed by using the identical CHOP antibody. (D) Analysis of average arbitrary units (AU) of CHOP in DIA. Data in B and E are presented as mean±S.E; *, p<0.05; **, p<0.01 CON versus ALS. (E) Same as A, protein isolated from CON and ALS mice cardiac muscle (HRT) and western blot was performed by using the identical CHOP antibody.

FIG. 17 shows caspase-12 is activated in skeletal muscle of G93A*SOD1 ALS mice. Western blot image with total protein loading control of superficial gastrocnemius (A), diaphragm (B) and cardiac muscle (C) from wild type (CON) and transgenic G93A*SOD1 (ALS) mice using specific antibody to caspase-12. Three postnatal ages are examined as follows: early pre-symptomatic (70 d; n=3 each for CON and ALS), late pre-symptomatic (90 d; n=5 each for CON and ALS), and symptomatic (120-140 d; n=3 each for CON and ALS) mice.

FIG. 18 shows caspase-12 in superficial gastrocnemius of transgenic G93A*SOD1 mice. Western blotting of soluble extracts of superficial gastrocnemius from wild type (CON) mice and transgenic G93A*SOD1 (ALS) mice and mice using specific antibody to caspase-12. Symptomatic (120-124 d) is examined

FIG. 19 shows p-eIF2α is up-regulated in skeletal muscle of G93A*SOD1 ALS mice. (A) Protein was isolated from different ages of wild-type (CON) and transgenic G93A*SOD1 (ALS) superficial gastrocnemius and western blot was performed by using antibody specific for eIF2α. Total protein was used as the loading control. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. (B) Analysis of ratio of phosphor-eIF2α to total eIF2α. Data in B are presented as mean±S.E; *, p<0.05; **, p<0.01 CON versus ALS.

FIG. 20 shows total p70S6K and phospho-p70S6K in skeletal muscle of G93A*SOD1 ALS mice. (A) Protein was isolated from different ages of wild-type (CON) and transgenic G93A*SOD1 (ALS) superficial gastrocnemius (SP-GAS) and western blot was performed by using antibody specific for p70S6K and phosphop70S6K. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. Quantification of Total p70S6K (B) and the ratio of phospo/Total p70S6K (C) in SP-GAS determined by densitometry and expressed as arbitrary units (AU). Similar data shown for deep gastrocnemius (DP-GAS): (D) raw western blot image and quantitative data for Total p70S6K (E) and the ratio of phospho to Total p70S6K (F). Average data represent mean±S.E; *, p<0.05; CON versus ALS.

FIG. 21 shows total Akt and phosphoAkt in skeletal muscle of G93A*SOD1 ALS mice. (A) Protein was isolated from different ages of wild-type (CON) and transgenic G93A*SOD1 (ALS) superficial gastrocnemius (SP-GAS) and western blot was performed by using antibody specific for Akt and phosphoAkt. Three postnatal ages were examined as follows: early pre-symptomatic (70 d; n=3 each for CON and ALS), late pre-symptomatic (90 d; n=5 each for CON and ALS), and symptomatic (120-140 d; n=3 each for CON and ALS) mice. Quantification of Total Akt (B) and the ratio of phospo/Total Akt (C) in SP-GAS determined by densitometry and expressed as arbitrary units (AU). Similar data shown for deep gastrocnemius (DP-GAS): (D) raw western blot image and quantitative data for Total Akt (E) and the ratio of phospho to Total Akt (F). Average data represent mean±S.E; *, p<0.05; CON versus ALS.

FIG. 22 shows differences in gastrocnemius muscle mass and muscle mass index in control (CON) and G93A*SOD1 (ALS) mice. Gastrocnemius muscle mass shown as absolute muscle mass (left) in grams (g) and expressed relative to animal body weight (right) in mg/g (n=4 per group). Bars represent mean±SEM. * p<0.05 vs. CON-Veh; a p<0.10 vs. CON-Veh; b p<0.12 vs. ALS-Veh.

FIG. 23 shows differences in muscle function in control (CON) and G93A*SOD1 (ALS) mice. Muscle function assessed by grip test measured in seconds (s) (n=4 per group). Grip test time in ALS-Veh and ALS-Gin was significantly lower than CON-Veh (p<0.05) but differences between ALS-Veh and ALS-Gin did not reach significance (p<0.10). Bars represent mean±SEM. * p<0.05 vs. CON-Veh; # p<0.10 vs. ALS-Veh.

FIG. 24 shows differences in stride length in control (CON) and G93A*SOD1 (ALS) mice. Stride length measured in centimeters (cm) between front and back limb. Stride length in ALS-Veh and ALS-Gin was significantly shorter than CON-Veh (p<0.05) but differences between ALS-Veh and ALS-Gin did not reach statistical significance. There was a trend for differences between ALS-Gin and ALS-Veh treated mice. Bars represent mean±SEM. *p<0.05 vs. CON-Veh; # p=0.13 vs. ALS-Veh.

FIG. 25 shows resting and peak Fura-2 ratios in control (CON) and G93A*SOD1 (ALS) mice. Resting Fura-2 ratios (left) and peak tetanic Fura-2 ratios (right) across the range of stimulation frequencies measured in single muscle fibres. There was a significant increase in resting Fura-2 ratio in ALS-Veh vs. CON-Veh and a tendency for Fura-2 ratio to be lower in ALS-Gin vs. ALS-Veh. Peak Fura-2 ratio (10 Hz) was higher in single muscle fibres from ALS-Veh treated compared to CON-Veh fibres. There was a trend for differences between fibres from CON-Veh and ALS-Gin at 10, 100, 120, and 150 Hz. Bars represent mean±SEM. (CON, n=28 fibres; ALS Gingerol, n=39; ALS Veh, n=39) *p<0.05 vs. CON-Veh; # p=0.13 vs. ALS-Veh.

FIG. 26 shows Ca²⁺ decay time in control (CON) and G93A*SOD1 (ALS) mice. Time to clear 75% of Ca²⁺ was higher in ALS-Veh single muscle fibres compared to CON-Veh fibres. There was a trend for single fibres from ALS-Gin to have a faster decay time compared to those from ALS-Veh mice. Bars represent mean±SEM (CON, n=53; ALS-Veh, n=92; ALS-Gin, n=75). *p<0.05 vs. CON-Veh; # p<0.11 vs. ALS-Veh.

FIG. 27 SERCA1 protein expression in gastrocnemius muscle of control (CON) and G93A*SOD1 (ALS) mice. A) Western blot data showing 4 of the 5 animal sets analyzed: CON-Veh, ALS-Veh and ALS-Gin. The Coomassie blue stained gel is shown to confirm equal loading of total protein in all lanes. B) Average data for all groups is shown: CON (n=5), ALS-Veh (n=5) and ALS-Gin (n=5) for SERCA1 expression. *=p<0.05 vs. CON; #=p<0.05 vs. ALS-Veh.

FIG. 28 shows CHOP protein expression in gastrocnemius muscle of control (CON) and G93A*SOD1 (ALS) mice. A) Western blot data showing 4 of the 5 animal sets analyzed: CON-Veh, ALS-Veh and ALS-Gin. The Coomassie blue stained gel is shown to confirm equal loading of total protein in all lanes. B) Average data for all groups is shown: CON (n=5), ALS-Veh (n=5) and ALS-Gin (n=5) for CHOP expression. *=p<0.05 vs. CON; #=p<0.05 vs. ALS-Veh.

FIG. 29 shows schematic illustration of breeding scheme for genetic proof of concept study for the use of SERCA agonists to treat ALS. Male G93A*SOD1 and female αSkA-SERCA1 Tg mice will be cross-bred to obtain G93A*SOD1 mice that overexpress SERCA1. Pups will be weaned at day 21 (21 d) and genotyped. Beginning at 35 d mice will be evaluated for motor-co-ordination by rotarod running time and muscle function assessed by grip test beginning at 70 d. Symptom onset and lifespan will be evaluated. At end of lifespan (˜120-140 d), tissues will be harvested for evaluation of motoneuron integrity (innervated vs. denervated neuromuscular junctions), cellular mechanisms of contractile function (Ca²⁺ handling, Ca²⁺ clearance) and skeletal muscle cellular function (activation of apoptosis and total cellular redox stress).

FIG. 30 shows in (A) a western blot of PERK, IRE1α, GRP78/BiP and PDI protein levels in wild-type mice administered vehicle (CON-Veh), ALS mice administered vehicle (ALS-Veh), and ALS mice administered 6-gingerol (ALS-Gin), in which GAPDH protein was used as a loading control; (B) a bar graph depicting the average data for PERK protein levels from the western blot in (A); (C) a bar graph depicting the average data for GRP78/BiP protein levels from the western blot in (A); (D) a bar graph depicting the average data for PDI protein levels from the western blot in (A); and (E) a bar graph depicting the average data for IRE1α protein levels from the western blot in (A).

FIG. 31 shows a bar graph plotting mice group versus Ca²⁺ ATPase maximum activity (μmol/g/min)

FIG. 32 shows in (A) a western blot of (β-actin protein from the skeletal muscle of wild-type mice (CON) and ALS mice at 70 days (d), 90 d, and 120 d-140 d, in which total protein was a loading control and (B) a western blot of α-tubulin protein from the skeletal muscle of wild-type mice (CON) and ALS mice at 120 d-140 d.

FIG. 33 shows a bar graph plotting the indicated protein level in human skeletal muscle from disease control (Disease CON) and SOD-1 AV4 sub-type of ALS (ALS SOD1-AV4) versus protein expression (arbitrary units, AU). This bar graph depicts the average data from the western blot of FIG. 34.

FIG. 34 shows a western blot of SERCA1, SERCA2, Akt, PDI, CHOP, β-actin, and α-tubulin proteins in human skeletal muscle from disease control (Disease CON) and SOD-1 AV4 sub-type of ALS (ALS SOD1-AV4).

FIG. 35 shows a schematic illustrating the effects of ALS and 6-gingerol treatment on ALS biomarkers. In each box, the white arrow represented the effects of 6-gingerol treatment on the biomarkers, while the solid black arrow represented the effects of ALS on the biomarkers.

### DETAILED DESCRIPTION

The present invention relates to markers for diagnosing amyotrophic lateral sclerosis (ALS) in a subject in need thereof. The markers can include factors and subfactors. The present invention also relates to a method of identifying factors and subfactors of ALS in the subject. The method includes obtaining a sample from the subject and measuring or detecting a level of the factor in the sample either alone or in combination with one, two, three, or more factors. The method also includes measuring or detecting a level of the subfactor in the sample alone, in combination with the factor, in combination with one, two, three, or more factors, in combination with one, two, three, or more subfactors, or any combination thereof.

The factor can be, for example, SERCA1, SERCA2, or GRP78/BiP. SERCA1 and SERCA2 protein levels can be significantly reduced or decreased in a subject suffering from ALS. BiP protein levels can be increased in the subject suffering from ALS. Accordingly, measurement of SERCA1, SERCA2, and/or BiP protein levels in the sample obtained from the subject can allow for the detection of ALS in the subject both before and after the onset of clinical symptoms of ALS. Detection of ALS can further be indicated by the measurement of one, two, three, or more subfactors in combination with the factor.

The present invention further relates to a method for diagnosing ALS in the subject and to a method for monitoring the efficacy of a treatment of ALS in the subject. Such methods can utilize the method of identifying factors and subfactors described above. The method of diagnosing ALS can compare a level of the factor (e.g., SERCA1, SERCA2, and BiP) measured in the sample obtained from the subject and a level of the factor measured in a control sample to determine if the subject is suffering from ALS. Additionally, the method of diagnosing ALS can compare a level of the subfactor in the sample obtained from the subject and a level of the subfactor in the control sample to further determine if the subject is suffering from ALS. Similar to the method of diagnosing ALS, the method of monitoring can compare levels of the factor before and after treatment to evaluate the efficacy of the treatment in the subject. The method of monitoring can compare levels of the subfactor before and after treatment to further evaluate the efficacy of the treatment.

The present invention relates to a method for treatment of ALS in the subject. The method can include administering a composition comprising a therapeutically effective amount of an agent. The agent may be 6-gingerol. 6-gingerol can significantly restore or increase the level of SERCA1 protein in the subject and increase SR Ca²⁺ ATPase activity. 6-gingerol can also decrease or reduce levels of apoptotic and/or stress factors, for example, CHOP, PERK, BiP, and IRE1α, in the subject. 6-gingerol can further increase or restore muscle mass and function in the subject suffering from ALS.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Nucleic acids” as used herein can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine, and isoguanine. Nucleic acids can be obtained by isolation or extraction methods, by chemical synthesis methods or by recombinant methods.

A “peptide,” “protein,” or “polypeptide” as used herein can mean a linked sequence of amino acids and can be natural, synthetic, or a modification or combination of natural and synthetic.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Variants can be a fragment thereof. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

The term “subject” or “patient” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc)) and a human. In some embodiments, the subject or patient may be a human or a non-human. The subject or patient may be undergoing other forms of treatment. In some embodiments, the subject or patient may be a human subject at risk for developing or already having ALS.

The term “control sample” or “control” as used herein means a sample or specimen taken from a subject, or an actual subject who does not have ALS, or is not at risk of developing ALS.

The term “sample,” “test sample,” “specimen,” “biological sample,” “sample from a subject,” or “subject sample” as used herein interchangeably, means a sample or isolate of blood, tissue, urine, serum, plasma, amniotic fluid, cerebrospinal fluid, placental cells or tissue, endothelial cells, leukocytes, or monocytes, can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

The term also means any biological material being tested for and/or suspected of containing an analyte of interest such as SERCA1, SERCA2, or BiP. The sample may be any tissue sample taken or derived from the subject. In some embodiments, the sample from the subject may comprise protein. In some embodiments, the sample from the subject may comprise nucleic acid. Any cell type, tissue, or bodily fluid may be utilized to obtain a sample. Such cell types, tissues, and fluid may include sections of tissues such as biopsy (such as muscle biopsy) and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include muscle tissue or fibres, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing muscle tissue or fibre, urine, blood, serum and plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

“Treat”, “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The term “effective dosage” as used herein means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual.

### 1. METHOD OF IDENTIFYING FACTORS AND SUBFACTORS OF ALS

Provided herein is a method of identifying factors and subfactors of ALS in a subject in need thereof. The method includes obtaining a sample from the subject and measuring or detecting a level of the factor in the sample either alone or in combination with one, two, three, or more factors. The method also includes measuring or detecting a level of the subfactor in the sample alone, in combination with the factor, in combination with one, two, three, or more factors, in combination with one, two, three, or more subfactors, or any combination thereof. In some embodiments, the level of the factor can be measured or detected in combination with the subfactor. In other embodiments, the level of the factor can be measured or detected in combination with one, two, three, or more subfactors.

A change in the level of the factor in the sample obtained from the subject relative to a control sample identifies the factor of ALS, thereby indicating that the subject is suffering from ALS. The change in the level of the factor can be an increase in the level of or a presence of the factor in the sample obtained from the subject. The change in the level of the factor can be an increase in or an up-regulation of the expression or activity of the factor in the sample obtained from the subject. Alternatively, the change in the level of the factor may be a decrease in the level of or an absence of the factor in the sample obtained from the subject. The change in the level of the factor can be a decrease in or a down-regulation of the expression or activity of the factor in the sample obtained from the subject.

A change in the level of the subfactor in the sample obtained from the subject relative to the control sample identifies the subfactor of ALS, thereby further indicating that the subject I suffering from ALS. The change in the level of the subfactor can be an increase in the level of or a presence of the subfactor in the sample obtained from the subject. The change in the level of the subfactor can be an increase in or an up-regulation of the expression or activity of the subfactor in the sample obtained from the subject. Alternatively, the change in the level of the subfactor may be a decrease in the level of or an absence of the subfactor in the sample obtained from the subject. The change in the level of the subfactor can be a decrease in or a down-regulation of the expression or activity of the subfactor in the sample obtained from the subject.

a. Factor

The method can identify one, two, three, or more factors of ALS alone or in combination in the sample obtained from the subject in need thereof. The method can measure or detect the change in the level of the factor in the sample alone, in combination with one, two, three, or more factors, in combination with one, two, three, or more subfactors, or any combination thereof. The method can also measure or detect the change in the level of the factor in the sample alone or in combination with one, two, three, or more subfactors.

The factor can be a nucleic acid sequence, an amino acid sequence, an ion, or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The ion can be a cation (e.g., Ca²⁺).

(1) SERCA 1 and SERCA 2

The factor can be a Sarcoplasmic Reticulum (SR)/Endoplasmic Reticulum (ER) Ca²⁺ (SERCA) pump or transporter. SERCA pumps hydrolyze ATP to actively transport or pump Ca²⁺ into the lumen of the sarcoplasmic reticulum for storage and to reduce Ca²⁺ levels in the cytoplasm. Cytoplasmic Ca²⁺ levels need to be reduced to maintain cellular function after an influx of Ca²⁺ into the cytoplasm in response to events such as calcium-mediated signal transduction and polarization of the cell membrane.

Three paralogs of SERCA exist in vertebrates, SERCA1, SERCA2, and SERCA3, which are alternatively spliced to produce more than 10 isoforms. SERCA1 isoforms are expressed in fast-twitch skeletal muscle. The SERCA2 gene produces SERCA2a and SERCA2b isoforms. The SERCA2a isoform is found in cardiac and slow-twitch skeletal muscle while the SERCA2b isoform is ubiquitously expressed at various levels across cell types. SERCA3 can be found in multiple cell types, for example, from the hematopoietic system, and exocrine and endocrine glands.

SERCA1 protein levels can be decreased in the sample obtained from the subject relative to the control sample, thereby identifying SERCA1 as a factor of ALS in the subject. In some embodiments, SERCA1 protein levels can be decreased about 40% to about 70% in the sample obtained from the subject. In other embodiments, SERCA1 protein levels can be decreased about 46% to about 66% in the sample obtained from the subject. In still other embodiments, SERCA1 protein levels can be decreased about 46%, 47%, 48%, 49%, 50%, 51%, 52%, 53%, 54%, 55%, 56%, 57%, 58%, 59%, 60%, 61%, 62%, 63%, 64%, 65%, or 66% in the sample obtained from the subject. Accordingly, a decrease in or a down-regulation of SERCA1 protein levels in the sample obtained from the subject relative to the control sample can be an indicator that the subject is suffering from ALS.

SERCA2 protein levels can also be decreased in the sample obtained from the subject relative to the control sample, thereby identifying SERCA2 as a factor of ALS in the subject. In some embodiments, SERCA2 protein levels can be decreased about 65% to about 99% in the sample obtained from the subject. In other embodiments, SERCA2 protein levels can be decreased about 75% to about 99% in the sample obtained from the subject. In still other embodiments, SERCA2 protein levels can be decreased about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 98%, or 99% in the sample obtained from the subject. Accordingly, a decrease in or a down-regulation of SERCA2 protein levels in the sample obtained from the subject relative to the control sample can be an indicator that the subject is suffering from ALS.

SERCA2 mRNA levels can be increased in the sample obtained from the subject relative to the control sample, thereby further identifying SERCA2 as a factor of ALS in the subject. Accordingly, an increase in or an up-regulation of SERCA2 mRNA levels in the sample obtained from the subject relative to the control sample can be an indicator that the subject is suffering from ALS.

(2) GRP78/BiP

The factor can be immunoglobin binding protein (GRP78/BiP). GRP78/BiP is an ER chaperone involved in the unfolded protein response (UPR). GRP78/BiP prevents aggregation of protein kinase RNA-activated-like ER kinase (PERK), inositol-requiring kinase-1 alpha (IRE1α), and activating transcription factor 6 (ATF6). GRP78/BiP, however, preferentially binds to misfolded proteins. Accordingly, in the presence of misfolded proteins, GRP78/BiP no longer prevents aggregation of PERK, IRE1α, and ATF6, which launches or induces the ER stress response. Induction of the ER stress response up-regulates GRP78/BiP expression.

GRP78/BiP protein levels can increased be in the sample obtained from the subject relative to the control sample, thereby identifying GRP78/BiP as a factor of ALS in the subject. GRP78/BiP protein levels can be increased in the sample obtained from the subject before the onset of clinical symptoms of ALS in the subject. Accordingly, an increase in GRP78/BiP expression or protein level in the sample obtained from the subject relative to the control sample can be an early indicator (i.e., before the onset of clinical symptoms) that the subject is suffering from ALS. Additionally, GRP78/BiP protein levels can be increased in the sample obtained from the subject after the onset or appearance of clinical symptoms of ALS in the subject. Accordingly, an increase in GRP78/BiP expression or protein level in the sample obtained from the subject relative to the control sample can be an indicator that the subject is suffering from ALS.

b. Subfactors

The method can identify one, two, three, or more subfactors alone, in combination, or in combination with the factor described above. The method can measure or detect the change in the level of the subfactor alone, in combination with one, two, three, or more subfactors, in combination with one, two, three, or more factors, or any combination thereof.

The subfactor can be a nucleic acid sequence, an amino acid sequence, an ion, or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The ion can be a cation (e.g., Ca²⁺).

(1) CnA Activity

The subfactor can be calcineurin (CnA). CnA is a serine/threonine kinase regulated by Ca²⁺/Calmodulin. CnA is a heterodimer including a calmodulin binding catalytic subunit and a Ca²⁺ binding regulatory subunit. Increases in intracellular calcium levels ([Ca²⁺]ᵢ) allow calmodulin to bind Ca²⁺, and the Ca²⁺/calmodulin complex binds the regulatory subunit of CnA, thereby activating CnA. Activation of CnA causes translocation of NFAT from the cytoplasm to the nucleus, and activation of slow fibre-type-specific and oxidative gene expression programs.

CnA activity, as measured by release of inorganic phosphate (Pᵢ), can be increased in the sample obtained from the subject relative to the control sample, thereby identifying CnA as a subfactor of ALS. In some embodiments, CnA activity can be increased about 0.64 fold to about 20 fold in the sample obtained from the subject. In other embodiments, CnA activity can be increased about 1 fold to about 12 fold in the sample obtained from the subject. In still other embodiments, CnA activity can be increased about 1 fold, 2 fold, 3 fold, 4 fold, 5 fold, 6 fold, 7 fold, 8 fold, 9 fold, 10 fold, 11 fold, or 12 fold in the sample obtained from the subject. Accordingly, an increase in or an up-regulation of CnA activity in the sample obtained from the subject relative to the control sample can be an indicator that the subject is suffering from ALS.

(2) NFAT Localization

The subfactor can be NFAT. NFAT can promote transcription of slow type-specific genes, for example, slow isoforms of myosin heavy chain and troponin I. NFAT moves from the cytoplasm to the nucleus in response to CnA activity, which in turn is activated by an increase in [Ca²⁺]ᵢ.

NFAT levels can be increased in the nuclear fraction of the sample obtained from the subject relative to the control sample, thereby identifying NFAT as a subfactor of ALS in the subject. NFAT levels can be decreased in the cytosolic fraction of the sample obtained from the subject relative to the control sample, thereby further identifying NFAT as a subfactor of ALS in the subject. Accordingly, a change in the cellular localization of NFAT in the sample obtained from the subject relative to the control sample can be an indicator that the subject is suffering from ALS.

(3) Intracellular Calcium Levels

The subfactor can be intracellular calcium levels ([Ca²⁺]ᵢ). [Ca²⁺]ᵢ in a muscle fibre can increase in response to stimulation or tetanus. To maintain cellular function, Ca²⁺ can then be removed from the cytoplasm by transporters or pumps such as the above described SERCA pump to return [Ca²⁺]ᵢ to pre-tetanus levels.

Resting [Ca²⁺]ᵢ can be increased in the sample obtained from the subject relative to the control sample, thereby identifying [Ca²⁺]ᵢ as a subfactor of ALS in the subject. In some embodiments, resting [Ca²⁺]ᵢ can be increased about 0.9 fold to about 18 fold in the sample obtained from the subject. In other embodiments, resting [Ca²⁺]ᵢ can be increased about 1 fold to about 12 fold in the sample obtained from the subject. In still other embodiments, resting [Ca²⁺]ᵢ can be increased about 1 fold, 2 fold, 3 fold, 4 fold, 5 fold, 6 fold, 7 fold, 8 fold, 9 fold, 10 fold, 11 fold, or 12 fold in the sample obtained from the subject. Accordingly, an increase in or an up-regulation of resting [Ca²⁺]ᵢ in the sample obtained from the subject relative to the control sample can be an indicator that the subject is suffering from ALS. Such an increase in resting [Ca²⁺]ᵢ in the sample obtained from the subject can be detected both prior to the onset of clinical symptoms of ALS and after the onset of clinical symptoms of ALS in the subject.

The return of [Ca²⁺]ᵢ to pre-tetanus levels can be delayed in the sample obtained from the subject relative to the control sample, thereby identifying return of [Ca²⁺]ᵢ to pre-tetanus levels as a subfactor of ALS in the subject. In some embodiments, the return to [Ca²⁺]ᵢ to pre-tetanus levels can be delayed about 5% to about 40% in the sample obtained from the subject. In other embodiments, the return of [Ca²⁺]ᵢ to pre-tetanus levels can be delayed about 13% to about 33% in the sample obtained from the subject. In still other embodiments, the return of [Ca²⁺]ᵢ to pre-tetanus levels can be delayed about 13%, 14%, 15%, 16%, 17%, 18%, 19%, 20%, 21%, 22%, 23%, 24%, 25%, 26%, 27%, 28%, 29%, 30%, 31%, 32%, or 33% in the sample obtained from the subject. Accordingly, a decrease in or a down-regulation of the return of [Ca²⁺]ᵢ to pre-tetanus levels in the sample obtained from the subject relative to the control sample can be an indicator that the subject is suffering from ALS.

(4) TnIs

The subfactor can be troponin I slow isoform (TnIs). TnIs can be expressed in slow-type muscle fibres. Slow-type muscle fibre gene expression programs can be induced or up-regulated by increased [Ca²⁺]ᵢ.

TnIs mRNA transcript levels can be increased in the sample obtained from the subject relative to the control sample, thereby identifying TnIs as a subfactor of ALS in the subject. In some embodiments, TnIs mRNA transcript levels can be increased about 2 fold to about 50 fold in sample obtained from the subject. In other embodiments, TnIs mRNA transcript levels can be increased about 9 fold to about 29 fold in the sample obtained from the subject. In still other embodiments, TnIs mRNA transcript levels can be increased about 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, or 29 fold in the sample obtained from the subject. Accordingly, an increase in or an up-regulation of TnIs mRNA transcript levels in the sample obtained from the subject relative to the control sample can be an indicator that the subject is suffering from ALS.

(5) Myoglobin

The subfactor can be myoglobin. Myoglobin can be expressed in oxidative type muscle fibres. Oxidative type muscle fibre gene expression programs can be induced or up-regulated by increased [Ca²⁺]ᵢ.

Myoglobin mRNA transcript levels can be increased in the sample obtained from the subject relative to the control sample, thereby identifying myoglobin as a subfactor of ALS. In some embodiments, myoglobin mRNA transcript levels can be increased about 25% to about 75% in the sample obtained from the subject. In other embodiments, myoglobin mRNA transcript levels can be increased about 40% to about 60% in the sample obtained from the subject. In still other embodiments, myoglobin transcript levels can be increased about 40%, 41%, 42%, 43%, 44%, 45%, 46%, 47%, 48%, 49%, 50%, 51%, 52%, 53%, 54%, 55%, 56%, 57%, 58%, 59%, or 60% in the sample obtained from the subject. Accordingly, an increase in or an up-regulation of myoglobin mRNA transcript levels in the sample obtained from the subject relative to the control sample can be an indicator that the subject is suffering from ALS.

(6) TnIf

The subfactor can be troponin I fast isoform (TnIf). TnIf can be expressed in fast-type muscle fibres. Fast-type muscle fibre gene expression programs can be down-regulated or inhibited by increased [Ca²⁺]ᵢ.

TnIf mRNA transcript levels can be decreased in the sample obtained from the subject relative to the control sample, thereby identifying TnIf as a subfactor of ALS in the subject. In some embodiments, TnIf mRNA transcript levels can be decreased about 45% to about 80% in the sample obtained from the subject. In other embodiments, TnIf mRNA transcript levels can be decreased about 52% to about 72% in the sample obtained from the subject. In still other embodiments, TnIf mRNA transcript levels can be decreased about 52%, 53%, 54%, 55%, 56%, 57%, 58%, 59%, 60%, 61%, 62%, 63%, 64%, 65%, 66%, 67%, 68%, 69%, 70%, 71%, or 72%. Accordingly, an decrease in or a down-regulation of TnIf mRNA transcript levels in the sample obtained from the subject relative to the control sample can be an indicator that the subject is suffering from ALS.

(7) MCK

The subfactor can be muscle creatine kinase (MCK). MCK can be expressed in fast-type muscle fibres. Fast-type muscle fibre gene expression programs can be down-regulated or inhibited by increased [Ca²⁺]ᵢ.

MCK mRNA transcript levels can be decreased in the sample obtained from the subject relative to the control sample, thereby identifying MCK as a subfactor of ALS in the subject. In some embodiments, MCK mRNA transcript levels can be decreased about 25% to about 75% in the sample obtained from the subject. In other embodiments, MCK mRNA transcript levels can be decreased about 40% to about 60% in sample obtained from the subject. In still other embodiments, MCK mRNA transcript levels can be decreased about 40%, 41%, 42%, 43%, 44%, 45%, 46%, 47%, 48%, 49%, 50%, 51%, 52%, 53%, 54%, 55%, 56%, 57%, 58%, 59%, or 60% in the sample obtained from the subject. Accordingly, a decrease in or a down-regulation of MCK mRNA transcript levels in the sample obtained from the subject relative to the control sample can be an indicator that the subject is suffering from ALS.

(8) GAPDH

The subfactor can be glyceraldehyde-3-phosphate dehydrogenase (GAPDH). GAPDH can be expressed in glycolytic type muscle fibres. Glycolytic type muscle fibre gene expression programs can be down-regulated or inhibited by increased [Ca²⁺]ᵢ.

GAPDH mRNA transcript levels can be decreased in the sample obtained from the subject relative to the control sample, thereby identifying GAPDH as a subfactor of ALS in the subject. In some embodiments, GAPDH mRNA transcript levels can be decreased about 25% to about 60% in the sample obtained from the subject. In other embodiments, GAPDH mRNA transcript levels can be decreased about 32% to about 52% in the sample obtained from the subject. In still other embodiments, GAPDH mRNA transcript levels can be decreased about 32%, 33%, 34%, 35%, 36%, 37%, 38%, 39%, 40%, 41%, 42%, 43%, 44%, 45%, 46%, 47%, 48%, 49%, 50%, 51%, or 52% in the sample obtained from the subject. Accordingly, a decrease in or a down-regulation of GAPDH mRNA transcript levels can be an indicator that the subject is suffering from ALS.

(9) Parvalbumin

The subfactor can be parvalbumin (PV). PV can buffer Ca²⁺ levels in muscle by binding Ca²⁺. PV can be more highly expressed in fast-type muscle fibres than slow-type muscle fibres.

PV proteins levels can be decreased in the sample obtained from the subject relative to the control sample, thereby identifying PV as a subfactor of ALS in the subject. In some embodiments, PV protein levels can be decreased about 20% to about 60% in the sample obtained from the subject. In other embodiments, PV protein levels can be decreased about 30% to about 50% in the sample obtained from the subject. In still other embodiments, PV protein levels can be decreased about 30%, 31%, 32%, 33%, 34%, 35%, 36%, 37%, 38%, 39%, 40%, 41%, 42%, 43%, 44%, 45%, 46%, 47%, 48%, 49%, or 50% in the sample obtained from the subject. Accordingly, a decrease in or a down-regulation of PV protein levels in the sample obtained from the subject relative to the control sample can be an indicator that the subject is suffering from ALS. Such a decrease in PV protein levels in the sample obtained from the subject can be detected both prior to the onset of clinical symptoms of ALS in the subject and after the onset of clinical symptoms of ALS in the subject.

For example, PV protein levels can be decreased in the sample obtained from the subject relative to the control sample before the onset of clinical symptoms of ALS in the subject. In some embodiments, PV protein levels can be decreased about 10% to about 50% in the sample obtained from the subject before the onset of clinical symptoms of ALS in the subject. In other embodiments, PV protein levels can be decreased about 20% to about 40% in the sample obtained from the subject before the onset of clinical symptoms of ALS in the subject. In still other embodiments, PV protein levels can be decreased about 20%, 21%, 22%, 23%, 24%, 25%, 26%, 27%, 28%, 29%, 30%, 31%, 32%, 33%, 34%, 35%, 36%, 37%, 38%, 39%, or 40% in the sample obtained from the subject even before the onset of clinical symptoms of ALS in the subject. Accordingly, a decrease or a down-regulation of PV protein levels in the sample obtained from the subject relative to the control sample before the onset of ALS clinical symptoms can be an indicator that the subject is suffering from ALS.

In another example, PV protein levels can be decreased in the sample obtained from the subject relative to the control sample after the onset of one or more clinical symptoms of ALS in the subject. In some embodiments, PV protein levels can be decreased about 30% to about 70% in the sample obtained from the subject after the onset of one or more clinical symptoms of ALS in the subject. In other embodiments, PV protein levels can be decreased about 40% to about 60% in the sample obtained from the subject after the onset of one or more clinical symptoms of ALS in the subject. In still other embodiments, PV protein levels can be decreased about 40%, 41%, 42%, 43%, 44%, 45%, 46%, 47%, 48%, 49%, 50%, 51%, 52%, 53%, 54%, 55%, 56%, 57%, 58%, 59%, or 60% in the sample obtained from the subject after the onset of one or more clinical symptoms of ALS in the subject. Accordingly, a decrease in or a down-regulation of PV protein levels in the sample obtained from the subject relative to the control sample after the onset of one or more clinical symptoms of ALS can be an indicator that the subject is suffering from ALS.

(10) PERK

The subfactor can be PERK. As discussed above, PERK can be an ER stress sensor involved in the unfolded protein response (UPR). PERK can be a transmembrane protein embedded in the ER with its N-terminus in the lumen of the ER and its C-terminus in the cytosol. PERK can aggregate with IRE1α and ATF6 when GRP78/BiP binds misfolded proteins. Aggregation of PERK, IRE1α, and ATF6 can activate the unfolded protein response, thereby causing up-regulation of GRP78/BiP and protein disulfide isomerase (PDI), and down-regulation of protein synthesis.

PERK protein levels can be increased in the sample obtained from the subject relative to the control sample, thereby identifying PERK as a subfactor of ALS. In some embodiments, PERK protein levels can be increased about 0.5 fold to about 15 fold in the sample obtained from the subject. In other embodiments, PERK protein levels can be increased about 1 fold to about 10 fold in the sample obtained from the subject. In still other embodiments, PERK protein levels can be increased about 1 fold, 2 fold, 3 fold, 4 fold, 5 fold, 6 fold, 7 fold, 8 fold, 9 fold, or 10 fold in the sample obtained from the subject. Accordingly, an increase in or an up-regulation of PERK protein levels in the sample obtained from the subject relative to the control sample can be an indicator that the subject is suffering from ALS. Such an increase in PERK protein levels in the sample obtained from the subject can be detected or measured both prior to the onset of clinical symptoms of ALS in the subject and after the onset of one or more clinical symptoms of ALS in the subject.

(11) IRE1α

The subfactor can be IRE1α. As discussed above, IRE1α can be an ER stress sensor involved in the unfolded protein response (UPR). IRE1α can be a transmembrane protein embedded in the ER with its N-terminus in the lumen of the ER and its C-terminus in the cytosol. IRE1α can aggregate with PERK and ATF6 when GRP78/BiP binds misfolded proteins. Aggregation of IRE1α, PERK, and ATF6 can activate the unfolded protein response, thereby causing up-regulation of GRP78/BiP and PDI, and down-regulation of protein synthesis.

IRE1α protein levels can be increased in the sample obtained from the subject relative to the control sample, thereby identifying IRE1α as a subfactor of ALS in the subject. In some embodiments, IRE1α protein levels can be increased about 0.5 fold to about 15 fold in the sample obtained from the subject. In other embodiments, IRE1α protein levels can be increased about 1 fold to about 10 fold in the sample obtained from the subject. In still other embodiments, IRE1α protein levels can be increased about 1 fold, 2 fold, 3 fold, 4 fold, 5 fold, 6 fold, 7 fold, 8 fold, 9 fold, or 10 fold in the sample obtained from the subject. Accordingly, an increase in or an up-regulation of IRE1α protein levels in the sample obtained from the subject relative to the control sample can be an indicator that the subject is suffering from ALS. Such an increase in IRE1α protein levels in the sample obtained from the subject can be detected or measured both prior to the onset of clinical symptoms of ALS in the subject and after the onset of one or more clinical symptoms of ALS in the subject.

(12) PDI

The subfactor can be protein disulfide isomerase (PDI). PDI is an ER chaperone that can be up-regulated in response to activation of the unfolded protein response, which was discussed in more detail above.

PDI protein levels can be increased in the sample obtained from the subject relative to the control sample, thereby identifying PDI as a subfactor of ALS in the subject. In some embodiments, PDI protein levels can be increased about 0.5 fold to about 20 fold in the sample obtained from the subject. In other embodiments, PDI protein levels can be increased about 1 fold to about 10 fold in the sample obtained from the subject. In still other embodiments, PDI protein levels can be increased about 1 fold, 2 fold, 3 fold, 4 fold, 5 fold, 6 fold, 7 fold, 8 fold, 9 fold, or 10 fold in the sample obtained from the subject. Accordingly, an increase in or an up-regulation of PDI protein levels in the sample obtained from the subject relative to the control sample can be an indicator that the subject is suffering from ALS. Such an increase or up-regulation of PDI protein levels can be detected or measured both prior to the onset of clinical symptoms of ALS in the subject and after the onset of one or more clinical symptoms of ALS in the subject.

(13) CHOP

The subfactor can be C/EBP homologous protein (CHOP). CHOP can be a signal of apoptosis that is induced or up-regulated by prolonged ER stress, for example, the unfolded protein response. ER stress can typically be a short term homeostatic mechanism necessary for cell survival, however, prolonged and severe ER stress can trigger apoptosis.

CHOP protein levels can be increased in the sample obtained from the subject relative to the control sample, thereby identifying CHOP as a subfactor of ALS in the subject. In some embodiments, CHOP protein levels can be increased about 0.5 fold to about 30 fold in the sample obtained from the subject. In other embodiments, CHOP protein levels can be increased about 1 fold to about 20 fold in the sample obtained from the subject. In still other embodiments, CHOP protein levels can be increased about 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, or 20 fold in the sample obtained from the subject. Accordingly, an increase in or an up-regulation of CHOP protein levels in the sample obtained from the subject relative to the control sample can be an indicator that the subject is suffering from ALS. Such an increase or up-regulation of CHOP protein levels can be detected or measured both prior to the onset of clinical symptoms of ALS in the subject and after the onset of one or more clinical symptoms of ALS in the subject.

(14) Caspase-12

The subfactor can be caspase-12. Caspase-12, specifically cleavage of caspase-12 into one or more smaller molecular weight proteins or peptides, can be a signal of apoptosis that is induced or up-regulated by prolonged ER stress (e.g., the unfolded protein response). ER stress can typically be a short term homeostatic mechanism necessary for cell survival, however, prolonged and severe ER stress can trigger apoptosis.

Caspase-12 cleavage can be increased in the sample obtained from the subject relative to the control sample, thereby identifying caspase-12 as a subfactor of ALS in the subject. Accordingly, an increase in or an up-regulation of caspase-12 cleavage in the sample obtained from the subject relative to the control sample can be an indicator that the subject is suffering from ALS. Such an increase or up-regulation of caspase-12 cleavage can be detected or measured both prior to the onset of clinical symptoms of ALS in the subject and after the onset of one or more clinical symptoms of ALS in the subject.

(15) p-eIF2α

The subfactor can be p-eIF2α. p-eIF2α can be a form of eIF2 in which the α subunit is phosphorylated, thereby preventing nucleotide exchange by the guanine exchange factor eIF2B. Nucleotide exchange by eIF2B is need for protein synthesis to continue. Accordingly, p-eIF2α effectively sequesters at least a portion of the pool of eIF2B in a cell, and thus, p-eIF2α causes a decrease in protein synthesis in the cell.

p-eIF2α protein levels can be increased in the sample obtained from the subject relative to a control sample, thereby identifying p-eIF2α as a subfactor of ALS in the subject. Accordingly, an increase in or up-regulation of p-eIF2α in the sample obtained from the subject relative to the control sample can be an indicator that the subject is suffering from ALS. Such an increase or up-regulation of p-eIF2α can be detected or measured both prior to the onset of clinical symptoms of ALS in the subject and after the onset of one or more clinical symptoms of ALS in the subject.

(16) β-Actin

The subfactor can be β-actin. Actin proteins may be involved in cell motility, structure, and integrity. Specifically, β-actin may be a major constituent of the contractile apparatus.

β-actin can form aggregates in the sample obtained from the subject relative to a control sample, thereby identifying β-actin aggregates as a subfactor of ALS in the subject. Accordingly, an increase in β-actin aggregates in the sample obtained from the subject relative to the control sample can be an indicator that the subject is suffering from ALS. Such an increase of β-actin aggregates can be detected or measured both prior to the onset of clinical symptoms of ALS in the subject and after the onset of one or more clinical symptoms of ALS in the subject.

Additionally, the presence and/or level of β-actin aggregates in the sample obtained from the subject can be an indicator of the severity of ALS in the subject. β-actin aggregates can increase with the severity of ALS.

(17) α-Tubulin

The subfactor can be α-tubulin. The tubulin family of proteins includes alpha-, beta-, gamma-, delta-, epsilon-, and zetα-tubulins. Alpha- and betα-tubulins form dimers that bind to GTP and in this GTP-bound state, assemble onto the positive (+)-end of microtubules. Upon hydrolysis of GTP to GDP, the dimer becomes less stable within the microtubule and may separate. Accordingly, the GTP-GDP cycle provides for the dynamic instability of microtubules.

α-tubulin can form aggregates (due to decreased solubility) in the sample obtained from the subject relative to a control sample, thereby identifying α-tubulin aggregates (i.e., decreased α-tubulin solubility) as a subfactor of ALS in the subject. Accordingly, an increase in α-tubulin aggregates (due to decreased α-tubulin solubility) in the sample obtained from the subject relative to the control sample can be an indicator that the subject is suffering from ALS. Such an increase in α-tubulin aggregates (due to the decrease in α-tubulin solubility) can be detected or measured both prior to the onset of clinical symptoms of ALS in the subject and after the onset of one or more clinical symptoms of ALS in the subject.

Additionally, the presence and/or level of α-tubulin aggregates (i.e., decreased a-tubulin solubility) in the sample obtained from the subject can be an indicator of the severity of ALS in the subject. α-tubulin solubility decreases with the severity of ALS, and thus, a-tubulin aggregates increase with the severity of ALS.

(18) Other Subfactors

The subfactor can be p70S6K (i.e., phosphorylated, unphosphorylated, or the combination thereof. The subfactor can also be Akt (i.e., phosphorylated, unphosphorylated, or the combination thereof). The subfactor can further be, but is not limited to, fructose-biphosphate aldolase A, isoform 3 of coiled-coil domain-containing protein 91, fatty acid-binding protein, cDNA FLJ54108, isoform 2 of ankyrin repeat domain-containing protein 2, isoform 2 of regucalcin, cDNA FLJ54106, a phophorylase, BTB/POZ domain-containing protein KCTD11, leucine-rich repeat-containing protein 14, isoform 1 of transmembrane protein 132D, prothrombin, cDNA FLJ53099, creatine kinase M-type, creatine kinase, isoform 1 of coiled-coil domain-containing protein C6orf199, cytochrome c oxidase subunit 4/isoform 1, ACTA2 protein, ATPase, protein tyrosine phosphatase, L-lactate dehydrogenase, uncharacterized protein GPKOW, and/or L-lactate dehydrogenase.

### 2. METHOD OF DIAGNOSING ALS

Also provided herein is a method of diagnosing ALS in a subject in need thereof. The method of diagnosing can apply the method of identifying factors and subfactors of ALS described above to determine if the subject is suffering from ALS. The method of diagnosing can include obtaining a sample from the subject, and measuring or detecting a level of one or more factors in the sample. The method of diagnosing can also include comparing the measured level of the one or more factors to a level of the factor in a control to determine if the subject is suffering from ALS. The method of diagnosing can further include measuring or detecting a level of one or more subfactors, and comparing the measured level of the one or more subfactors to a level of the subfactor in the control to determine if the subject is suffering from ALS.

### 3. METHOD OF DIAGNOSING THE SEVERITY OF ALS

Further provided herein is a method of diagnosing the severity of ALS in the subject in need thereof. The method of diagnosing the severity of ALS can apply the method of identifying factors and subfactors of ALS described above to determine the severity of ALS in the subject. The method of diagnosing the severity of ALS can include obtaining a sample from the subject, and measuring or detecting a level of one or more factors in the sample. The method of diagnosing the severity of ALS can also include comparing the measured level of the one or more factors to a level of the factor in a control to determine the severity of ALS in the subject.

The method of diagnosing the severity of ALS can further include measuring or detecting a level of one or more subfactors, and comparing the measured level of the one or more subfactors to a level of the subfactor in the control to determine the severity of ALS in the subject. In some embodiments, the one or more subfactors may be β-actin and/or α-tubulin, which are described above in more detail. As described above, β-actin aggregates may increase with the severity of ALS while α-tubulin solubility may decrease with the severity of ALS as evidenced by increased α-tubulin aggregates. Accordingly, the detected levels (i.e., the relative increase to the control) and/or presence of β-actin aggregates and/or α-tubulin aggregates may indicate the severity of ALS in the subject.

### 4. METHOD OF MONITORING EFFICACY OF TREATMENT OF ALS

Provided herein is a method of monitoring efficacy of treatment of ALS in a subject undergoing treatment of ALS in any form. The method of monitoring can apply the method of identifying factors and subfactors of ALS described above to determine if the treatment of ALS has a therapeutic effect in the subject. The method of monitoring can include obtaining a first sample from the subject before treatment has begun, and obtaining a second sample from the subject after treatment has begun. The levels of one or more factors can be measured or detected in the first and second samples to determine a first level and a second level of the one or more factors, respectively. The first and second levels of the one or more factors can be compared to determine if the second level is different or changed (e.g., higher or lower) from the first level, in which the difference indicates whether the ALS treatment has had a therapeutic effect in the subject.

The method of monitoring can also include measuring or detecting first and second levels of one or more subfactors in the first and second samples, respectively, and comparing the first and second levels of the one or more subfactors. If the second level of the one or more subfactors is different or changed (e.g., higher or lower) from the first level, the difference then further indicates whether the ALS treatment has had a therapeutic effect in the subject.

In some embodiments, a method for monitoring the efficacy of a treatment for amyotrophic lateral sclerosis (ALS) in a subject, the method comprising obtaining a first sample from the subject before the treatment and a second sample from the subject during or after treatment; measuring a first level of a protein in the first sample and a second level of the protein in the second sample, wherein the protein is selected from the group consisting of SERCA1 and PV; or the protein is selected from the group consisting of CHOP, Caspase-12, PERK, BiP, IRE1α, and PDI; and comparing the first level of the protein and the second level of the protein, wherein a second level of the protein during or after treatment of (b)(i) is higher than the first level of the protein of (b)(i) before treatment and is indicative of a therapeutic effect of the treatment in the subject; or a second level of the protein during or after treatment of (b)(ii) is lower than the first level of the protein of (b)(ii) before treatment and is indicative of a therapeutic effect of the treatment in the subject.

### 5. KITS

Also provided herein are kits for use with the methods disclosed herein. The kits can include reagents for detecting the factors and subfactors either alone or in any combination thereof. The reagents can be any of those reagents known in the art for immunoassays (e.g., ELISA, western blotting, immunoprecipitation (IP), immunohistochemistry, etc.) to detect the factors and subfactors. The reagents can also be any of those reagents known in the art for detecting nucleic acids, for example, polymerase chain reaction (PCR), reverse transcriptase-PCT (RT-PCR), northern blotting, quantitative RT-PCT (qRT-PCR), and so forth. The kits also include controls and instructions for how to use the kit.

### 6. METHOD OF TREATING ALS

Provided herein is a method for treating ALS in a subject in need thereof. The method includes administering a composition comprising a therapeutically effective amount of an agent.

a. Agent

In a subject suffering from ALS, the agent can alter the level or activity of one or more of the factors discussed above in the subject such that the level or activity of the one or more factors in a sample obtained from subject after treatment has begun is substantially the same as a level or activity of the one or more factors in a control sample. The agent can also alter the level or activity of one or more subfactors discussed above in the subject such that the level or activity of the one or more subfactors in the sample obtained from the subject after treatment has begun is substantially the same as a level or activity of the one or more subfactors in the control sample.

In some embodiments, the agent can increase SERCA1 protein levels in the subject. In other embodiments, the agent can decrease CHOP, PDI, PERK, BiP, and/or IRE1α protein levels in the subject. In still other embodiments, the agent can increase skeletal muscle function in the subject. In some embodiments, the agent can increase sarcoplasmic reticulum (SR) Ca²⁺ ATPase activity.

(1) 6-gingerol

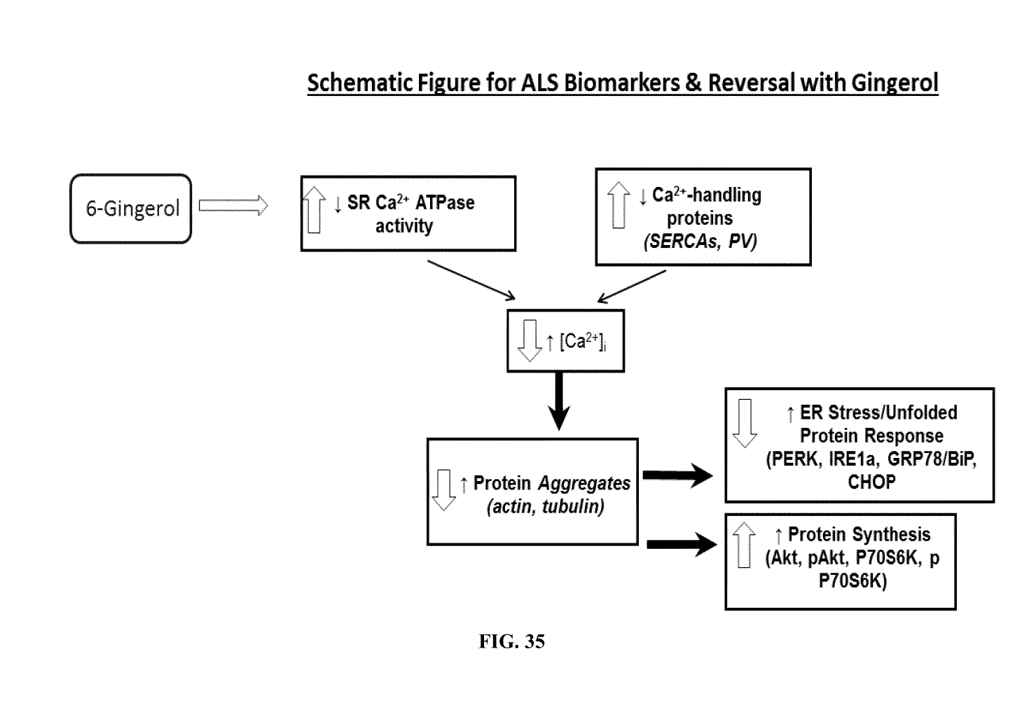

The agent can be 6-gingerol. The effects of 6-gingerol are summarized in FIG. 35. In the subject, 6-gingerol can increase SR Ca²⁺ ATPase activity, increase levels of Ca²⁺ handling proteins, decrease [Ca²⁺]ᵢ, decrease levels of protein aggregates (e.g., β-actin aggregates and α-tubulin aggregates), decrease levels of proteins involved in ER stress and/or the unfolded protein response (e.g., PERK, IRE1α, PDI, BiP, and CHOP), and increase the levels of proteins involved in protein synthesis (e.g., Akt, pAkt, P70S6K, and pP70S6K) (FIG. 35).

6-gingerol can increase SERCA Ca²⁺ ATPase activity, thereby causing increased reuptake of Ca²⁺ into the sarcoplasmic reticulum. 6-gingerol can increase SERCA1 protein levels in the subject. 6-gingerol can decrease CHOP protein levels in the subject. 6-gingerol can also decrease PDI, PERK, BiP, and IRE1α, protein levels in the subject.

6-gingerol can improve skeletal motor function in the subject. In some embodiments, 6-gingerol can improve muscle mass about 5% to about 40% in the subject. In other embodiments, 6-gingerol can improve muscle mass about 10% to about 30% in the subject. In still other embodiments, 6-gingerol can improve muscle mass about 10%, 11%, 12%, 13%, 14%, 15%, 16%, 17%, 18%, 19%, 20%, 21%, 22%, 23%, 24%, 25%, 26%, 27%, 28%, 29%, or 30%.

The present invention has multiple aspects, illustrated by the following non-limiting examples.

### 7. EXAMPLES

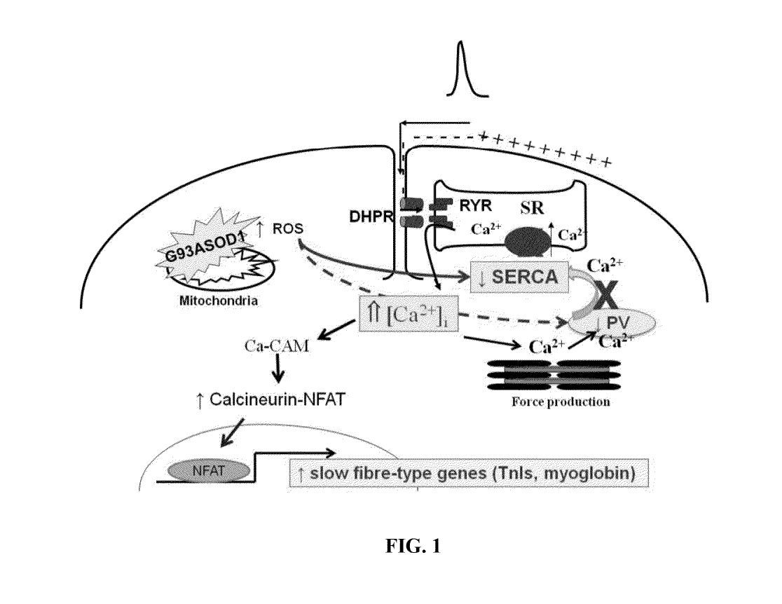

As illustrated in FIG. 1 and shown in the Examples below, excitation of skeletal muscle by the motoneuron can lead to depolarization of the muscle sarcolemmal and transverse tubule membranes, activation of the voltage-sensing dihydropyridine receptor (DHPR) and Ca²⁺ release from the sarcoplasmic reticulum (SR) via the ryanodine receptors (RYR). This release of Ca²⁺ can elevate intracellular free Ca²⁺ concentration ([Ca²⁺]ᵢ) and activate muscle cross-bridges to produce force. Ca²⁺ removal and thus muscle relaxation can be due to buffering by the high affinity Ca²⁺ binding protein parvalbumin (PV) and Ca²⁺ removal by the SR/Endoplasmic Reticulum (ER) Ca²⁺ pump (SERCA). Neural activation can lead to repetitive Ca²⁺ transients, which both activate contraction and gene expression pathways. In the G93A*SOD1 mice, there can be a reduction in PV and SERCA pump expression leading to decreased Ca²⁺ removal following neural activation, increased resting and peak tetanic [Ca²⁺]ᵢ at low stimulation frequencies (i.e. during slow motoneuron input). This low amplitude but sustained increase in [Ca²⁺]ᵢ can activate the slow muscle fibre type and oxidative gene expression pathways, leading to a shift to slow and oxidative (red) fibres in fast muscles.

### Example 1

### Materials and Methods for Examples 2-6

Animals.

Control (C57BL/6 SJL hybrid) female and ALS (C57BL/6 SJL-Tg SOD1*G93A) male mice were obtained from Jax laboratories. Control (CON) and G93A*SOD1 ALS heterozygote mice were bred to establish a colony. Mice were weaned at 21 d and genotyped to determine whether they were wild-type (CON) or G93A*SOD1 transgenic (Tg) mice. Male and female Tg mice along with CON littermates were investigated at the pre-symptomatic ages of 70 d and 90 d and in symptomatic mice (i.e., mice having visible muscle weakness, hindlimb paralysis, and reduced mobility) at 120-140 d (see Table 1). Within these age-groups, 70 d represented an early pre-symptomatic and 90 d a late pre-symptomatic phase just prior to onset of overt symptoms. These age-groups were chosen because functional muscle deficits exist in 60 d old pre-symptomatic mice and in 3-4 mos. old symptomatic mice. At time of use, animals were euthanized by CO₂ inhalation followed by cervical dislocation. Tissues were harvested for immediate dissection to obtain single fibres from the flexor digitorum brevis (FDB) muscle or were quick frozen in liquid nitrogen for later analysis of muscle transcript and protein levels.

TABLE 1
Age and body weight of wild-type and G93A*SOD1
ALS transgenic mice at time of use

| | | Age at time of use | Body Weight |
|---|---|---|---|
| Age group | | (days) | (g) |
| 70 d | Wild-type | 69 ± 3 (n = 3) | 25.0 ± 4.9 |
| | G93A*SOD1 Tg | 70 ± 3 (n = 3) | 20.8 ± 3.0 |
| 90 d | Wild-type | 94 ± 3 (n = 3) | 21.8 ± 3.2 |
| | G93A*SOD1 Tg | 95 ± 4 (n = 3) | 19.3 ± 1.6 |
| 120- | Wild-type | 134 ± 6 (n = 5) | 29.4 ± 2.8 |
| 140 d | G93A*SOD1 Tg | 134 ± 6 (n = 5) | 17.7 ± 1.3 ** |

** p < 0.01 vs. wild-type

Single Muscle Fibre Isolation and E-C Coupling Measurements.

Intact single muscle fibres were obtained from the FDB by collagenase digestion. Briefly, strips of FDB muscle were placed in 0.2% collagenase (Worthington, Type 2) in Minimal Essential Media with 10% Fetal Bovine Serum and 1% penicillin/streptomycin (MEM/FBS media) and allowed to digest 4 hrs in a tissue culture incubator (37 degrees Celsius, 95% 0₂, 5% CO₂). After 4 hrs, fibres were triturated in MEM/FBS media and then left in the incubator overnight. Fibres were assessed for changes in e-c coupling within 24 hrs of isolation.

For e-c coupling measurements, fibres were loaded with 1 μM Fura-2 AM in MEM/FBS media for 15 min at room temperature. Fura-2 AM media was then removed by quick centrifugation and fibres were resuspended in fresh MEM/FBS media. Fibres loaded with Fura-2 were placed in a culture/stimulation chamber (Cell MicroControls) containing parallel electrodes on top of a Nikon TiU microscope. Once in the chamber, fibres were continuously perfused with stimulating tyrode (121 mM NaCl, 5 mM KCl, 1.8 mM CaCl₂, 0.5 mM MgCl₂, 0.4 mM NaH₂PO₄, 24 mM NaHCO₃, and 5.5 mM glucose) pH 7.3 when continuously bubbled with 95% O2/5% CO₂ using a Gilson Minipuls 3 peristaltic pump and vacuum pump. Intracellular Ca²⁺ levels were assessed by Fura-2 fluorescence ratio (ratio of excitation at 340 and 380 nm; emission at 510 nm) using the IonOptix Hyperswitch system, with the Hyperswitch enabling collection of ratiometric data at a frequency of 250 Hz. Ratios were converted to intracellular free Ca²⁺ concentration ([Ca²⁺]ᵢ). The Fura-2 ratio was calibrated in vivo using the Ca²⁺ ionophore A23187 and 10 mM EGTA or 1 mM CaCl₂, with Rₘᵢₙ and Rₘₐₓ determined to be 0.33 and 3.50, respectively. The K_{d} for Ca²⁺ for Fura-2 was 224 nM.

Fura-2 loaded single fibres were stimulated using 350 ms tetani, 0.5 ms pulse duration at 10, 30, 50, 70 and 100, 120 and 150 Hz stimulation frequencies (S48 Square Pulse Stimulator, GRASS Technologies) with one minute rest between frequencies. All single fibre data was collected at room temperature (23 degrees Celsius). Due to the variability in Fura-2 ratios between fibres, all other sources of variability (i.e., day to day variability) were reduced by obtaining fibres from one CON and one ALS Tg mouse on the same day and subsequently analyzing single fibre e-c coupling on the same day.

Assessment of Calcineurin Activity.

Activation of the CnA/NFAT pathway was assessed by measuring calcineurin enzyme activity using the Enzo Life Science Calcineurin Cellular Assay kit. Briefly, quadriceps muscle tissue was homogenized in lysis buffer (50 mM Tris pH 7.5, 0.1 mM EDTA, 0.1 mM EGTA, 1 mM DTT, 0.2% NP-40 with protease inhibitor cocktail (Enzo Life Sciences BML-KI103). Homogenates were desalted twice on Sephadex G25 columns (Roche Quick Spin Protein Columns G-25) and the desalted protein concentration assessed using the BCA protein kit (Thermo Scientific). Calcineurin activity was assayed according to the manufacturer's protocol with 5 μl homogenate in 25 μl 2× assay buffer (100 mM Tris pH7.5, 200 mM NaCl, 12 mM MgCl₂, 1 mM DTT, 0.05% NP-40, 1 mM CaCl₂ and 0.5 μM calmodulin), 10 μl RII phosphopeptide substrate and either 5 μl H₂O, 5 μl Vehicle (ETOH) or 5 μl Cyclosporin A. Additional background (no substrate) and positive control (human recombinant calcineurin) samples were assessed. A phosphate (Pi) standard curve (0.031-2 nmol Pi) was run in 1× assay buffer. The Rh peptide was used to initiate the reaction. After 5 min incubation, 100 μl Biomol Green was added to terminate the reaction and to allow colorimetric assessment of free Pi released by CnA activity. Calcineurin activity was calculated as the difference in Pi released per minute per mg protein in the absence versus the presence of Cyclosporin A. Assays were run in duplicate on 2 separate occasions to confirm differences between CON and ALS genotypes.

Muscle Protein and Transcript Analyses.

The superficial (SP) and deep (DP) portions of the gastrocnemius muscle (SP GAS and DP GAS, respectively) were used for analysis of Ca²⁺ regulatory protein levels by western blot. Anatomically, the gastrocnemius muscle is composed of a lateral and medial head, with a similar mixture of fibres types in the two heads: lateral gastrocnemius is 69% fast glycolytic (FG), 30% fast oxidative and glycolytic (FOG) and 1% slow oxidative (SO) fibres and medial gastrocnemius is 55% FG, 32% FOG and 8% SO. The muscle was divided by superficial vs. deep portions due to their glycolytic (white) vs. oxidative (red) differences, respectively. For muscle protein analysis, SP GAS and DP GAS samples were homogenized in lysis buffer (20 mM Hepes, pH 7.5, 100 mM NaCl, 1.5 mM MgCl2, 0.1% Triton X-100, 20% Glycerol) containing 1 mM DTT and a protease inhibitor cocktail (Complete mini EDTA-free Protease Inhibitor Cocktail, Roche). Protein levels were determined using a BCA protein kit (Thermo Scientific). Samples were then solubilized in loading buffer and denatured (5 min at 100 degrees Celsius). For assessment of changes in sarcoplasmic reticulum (SR)/endoplasmic reticulum (ER) Ca²⁺ (SERCA) pump, SERCA1 and SERCA2 protein levels were measured. Specifically, 15 μg protein was loaded on 8% gels and analyzed by polyacrylamide gel electrophoresis (PAGE). Levels of parvalbumin (PV) were assessed by loading 2.5 μg protein on 15% gels. Proteins were transferred to PVDF membrane and probed with antibodies for SERCA1 (Thermo Scientific; 1:1000), SERCA2 (Santa Cruz; 1:1000) and PV (Swant; 1:1000). Level of each respective protein was quantified by chemiluminescence using SuperSignal West Dura Chemiluminescent Substrate (Thermo Scientific) and imaged using a chemiluminescence imaging system (GeneGnome, Syngene). Protein expression levels are expressed as Arbitrary Units (AU).

Changes in the expression of fast and slow fibre type-specific genes were assessed in the mixed fast fibre type Tibialis Anterior (TA) muscle. The TA was chosen for determining changes in fibre type-specific gene expression patterns since it has 35% FG and 65% FOG fibres and undergoes fibre type shifts in response to physiological stimuli including functional overload. For gene transcript analyses, frozen TA muscle was homogenized in TriPure Reagent (Roche) and mRNA isolated as per manufacturers protocol. mRNA quantity and purity were assessed using a Nanodrop spectrophotometer and mRNA was diluted to 10 ng/μl. Transcript levels were analyzed using Reverse Transcriptase Polymerase Chain Reaction (RT-PCR) to assess changes in fibre type-specific gene expression. For SERCA2 only, gene expression was assessed using semi-quantitative RT-PCR with the following primers and conditions: Forward primer: TGC CTG GTG GAG AAG ATG AAT G (SEQ ID NO: 1); Reverse primer: CTG TTT GAC ACC AGG AGT CAT G (SEQ ID NO: 2); PCR cycle 95° C. 3 min, [94° C. 30 sec, 54° C. 1 min, 72° C. 1 min]×25 cycles, 72° C. 3 min. For all other genes, quantitative PCR was completed using validated TaqMan primer probe pairs (Applied Biosystems) and conditions optimized to ensure linearity of each gene through a 10-fold range. The transcripts used to assess fibre-type specific gene expression were: i) Troponin I slow (TnIs; Mm01295955_m1) as a marker of slow fibre-type specific genes; ii) Myoglobin (Mb; Mm00442969_m1) as a marker of oxidative genes; iii) Troponin I fast (TnIf; Mm01268884_g1) as a marker of fast fibre-type genes; iv) Muscle Creatine Kinase (MCK; Mm00432556_m1) as a marker of fast fibre-type and also anaerobic metabolism; v) Glyceraldehyde-3-phosphate dehydrogenase (GAPDH; Mm99999915_g1) as a marker of fast glycolytic fibre type gene. All Taqman target genes were run in a multiplexed assay with primer-probes for 18S. Each sample was analyzed in triplicate and average cycle threshold (Ct) used to calculate changes in target genes relative to 18S (internal control) and then changes in Tg muscle to the first CON muscle (CON1) on the 96 well plate using the ΔΔCt method. Fold changes in gene expression were calculated as 2^{ΔΔCt}.

Study Design and Statistical Analysis.

To assess the changes in e-c coupling in G93A*SOD1 muscle, 10-15 fibres were analyzed per mouse for a total of 30-50 fibres for each genotype at each time point. Values are expressed as mean±standard error (SE). To evaluate differences between CON and ALS Tg mice, data were analyzed using a 1-way ANOVA with between subject design and genotype as the independent variable. Comparisons were made for each age group separately and data expressed for ALS Tg mice relative to the CON mice at each time point. Tukey post-hoc analyses were used for all significant ANOVAs and p<0.05 used to determine statistical significance.

### Example 2

### Alterations in e-c Coupling in Intact Single Muscle Fibres of ALS Mice

E-C coupling facilitates communication between nerve and muscle. E-C coupling in the muscle of ALS mice was investigated to determine if e-c coupling is altered in ALS mice as compared to wild-type mice.

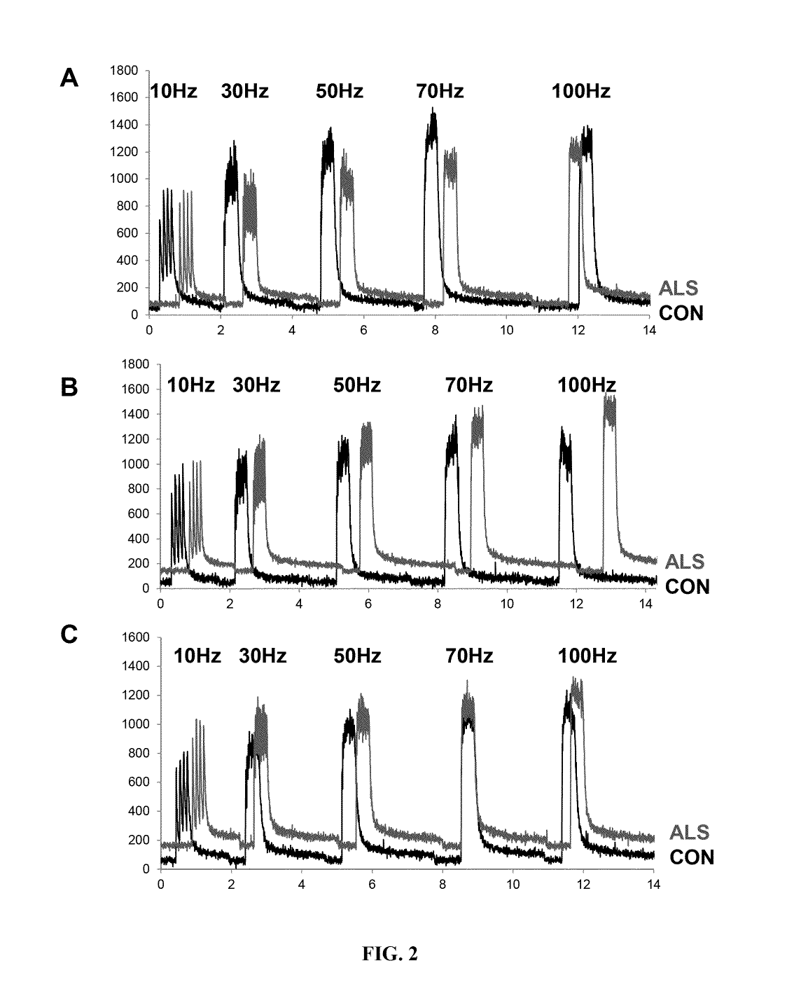

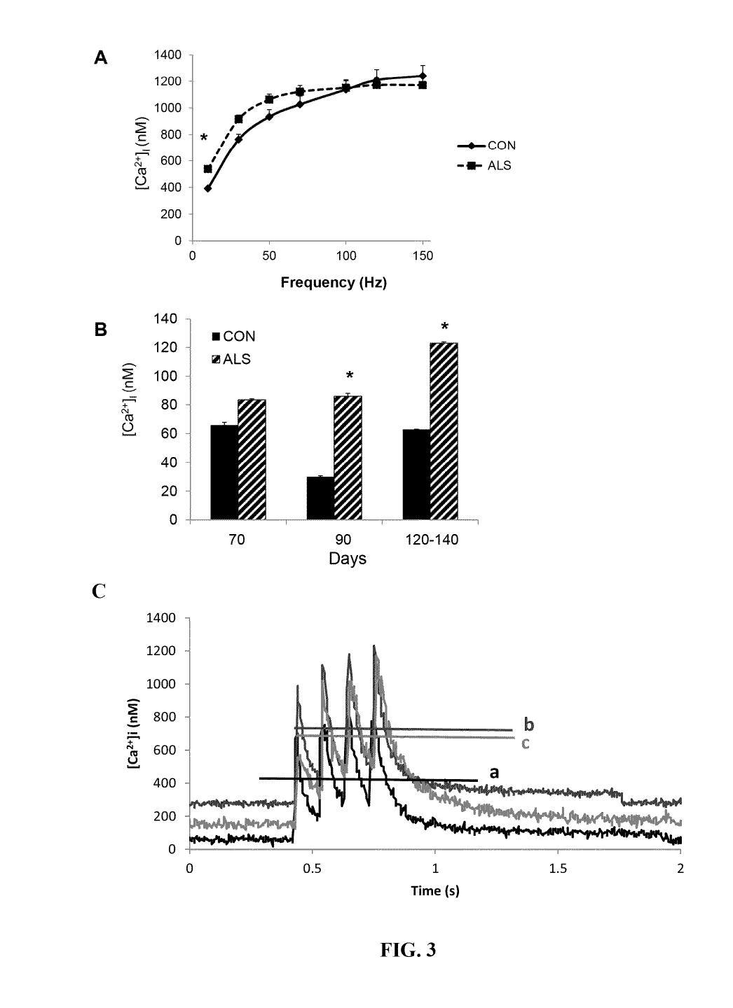

Specifically, single muscle fibres isolated by collagenase digestion retain an intact, polarized sarcolemmal membrane which can be depolarized by electrical field stimulation to activate the normal physiological process of e-c coupling and muscle contraction. These fibres were stimulated with a range of stimulation frequencies to cover the physiological firing frequencies for slow motor units with type I fibres (10, 30 Hz), fast fatigue-resistant motor units with type IIa fibres (50, 70 Hz), and fast fatiguable motor units with type IIb fibres (100, 120 and 150 Hz) (24). The [Ca²⁺]ᵢ during tetanic stimulation at each of these frequency ranges, are shown for representative fibres at 70 d (FIG. 2A), 91 d (FIG. 2B), and 134 d (FIG. 2C). At 70 d there were no differences in [Ca²⁺]ᵢ between single fibres isolated from CON and ALS Tg mice at any of the frequencies tested. However, at 91 d and 134 d, there were increases in [Ca²⁺]ᵢ at 10 Hz (FIGS. 2B and 2C). Average data for all fibres from 120-140 d old mice are shown in FIG. 3. The average steady state [Ca²⁺]ᵢ was significantly higher in ALS fibres at the lowest frequency assessed (10 Hz: 480±20 vs. 380±16 nM and 538±16 vs. 392±13 nM for ALS Tg vs. CON, at 90 d and 120-140 d, respectively; p<0.05) with the difference at 30 Hz not quite reaching statistical significance (915±32 vs. 762±40) for ALS Tg vs. CON at 120-140 d; p=0.135). These data indicate that ALS myofibres would experience a higher time-averaged [Ca²⁺]ᵢ at lower activation frequencies. Furthermore, assuming there are no changes in myofibrillar protein Ca²⁺ sensitivity or myofibrillar protein damage, these data indicate that force output at lower stimulation frequencies would be augmented.

While most fibres were able to respond to electrical stimulation, an increase in the percent of fibres that failed to maintain a plateau in the Fura-2 peak at higher stimulation frequencies was observed. This phenomenon is observed in a fraction of all muscle fibre preparations, but was greater in ALS compared to CON fibres. Out of a subset of fibres analyzed, 4/18 (23%) and 9/21 (43%) failed to maintain a peak at 100 Hz stimulation in CON compared to ALS, respectively. As a result, greater variability in peak [Ca²⁺]ᵢ in fibres from ALS mice was observed, especially at the 120-140 d time point. This variability may be related to disease severity or the magnitude of oxidative stress-induced modifications of proteins that regulate Ca²⁺ leak, Ca²⁺ release and Ca²⁺ removal. However, overall, there were no changes in peak tetanic [Ca²⁺]ᵢ at stimulation frequencies >10 Hz.

In contrast to the varied response in peak tetanic [Ca²⁺]ᵢ there was a consistent increase in resting [Ca²⁺]ᵢ in muscle fibres from ALS compared to CON mice. Significant differences were observed as early as 90 d and persisted at 120-140 d of age (FIG. 3B). At the late pre-symptomatic 90 d timepoint, resting [Ca²⁺]ᵢ increased 2.9-fold from 30±0.7 nM (CON) to 86±2.0 nM (ALS Tg) (p<0.05). At the symptomatic 120-140 d timepoint [Ca²⁺]ᵢ, increased 2.0-fold, from 63±0.2 nM (CON) to 123±0.9 nM (ALS) (p<0.05. Overall, these data show a 2-3-fold increase in resting [Ca²⁺]ᵢ in ALS fibres, indicating impaired Ca²⁺ handling and impaired intracellular Ca²⁺ regulation beginning at a pre-symptomatic phase of ALS disease.

In order to assess whether the rise in resting [Ca²⁺]ᵢ was due to an impairment of the SR Ca²⁺ pump, the time for the fall in [Ca²⁺]ᵢ during the return to baseline at the end of the tetanus for 50 and 100 Hz was examined. FIG. 3C shows raw data traces of 100 Hz Ca²⁺ transients on an expanded timescale, with the slower return to baseline in ALS vs. CON muscle fibre. In 120-140 d old mice, the time for [Ca²⁺]ᵢ to return to 75% of the pre-tetanus baseline was significantly longer in fibres from ALS mice (85.1±4.0 msec vs. 69.5±5.4 msec for ALS vs. CON fibres, p<0.05). Thus, it took ˜23% longer for Ca²⁺ to be removed from the cytoplasm, indicating an impairment of Ca²⁺ clearance mechanisms such as SERCA and mitochondrial Ca²⁺ pump proteins.

The above results showed that at late pre-symptomatic (i.e., 91 d) and symptomatic (i.e., 134 d) stages of ALS, [Ca²⁺]ᵢ was increased at 10 Hz in ALS fibres as compared to CON fibres. Additionally, the average steady state [Ca²⁺]ᵢ was significantly higher in ALS fibres at 10 Hz as compared to CON fibres. Accordingly, ALS fibres experience higher time-averaged [Ca²⁺]ᵢ at lower activation frequencies.

The above results also showed an increase in resting [Ca²⁺]ᵢ in ALS fibres as compared to CON fibres. As early as the late pre-symptomatic (i.e., 91 d) stage of ALS, an 2.9-fold increase in resting [Ca²⁺]ᵢ was observed in ALS fibres relative to CON fibres. Additionally, at the symptomatic (i.e., 120-140 d) stage of ALS, an 2.0-fold increase in resting [Ca²⁺]ᵢ was observed in ALS fibres relative to CON fibres. Accordingly, these data showed a 2-3-fold increase in [Ca²⁺]ᵢ in ALS fibres, which indicates impaired Ca²⁺ handling and regulation in the pre-symptomatic stage of ALS.

The above results further showed a significantly longer time for [Ca²⁺]ᵢ to return to pre-tetanus baseline in ALS fibres. Specifically, Ca²⁺ removal from the cytoplasm in ALS fibres took about 23% longer, indicating impairment in Ca²⁺ removal mechanisms (e.g., SERCA).

### Example 3

### Activation of the Calcineurin-NFAT-Dependent Transcriptional Pathway

The calcineurin-NFAT (CnA/NFAT) pathway is involved in calcium dependent signaling in muscle. The CnA/NFAT pathway was investigated in ALS mice by examining whether CnA enzyme activity and NFAT cellular localization (i.e., nucleus vs. cytoplasm) was altered in the skeletal muscle of ALS mice.

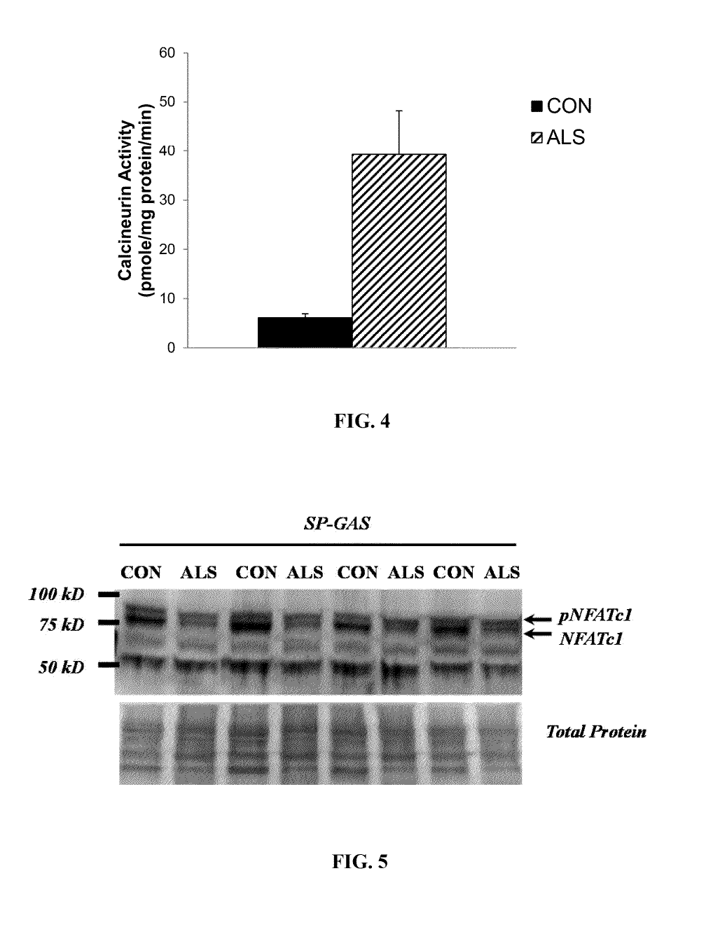

Specifically, to assess whether the above described increase in resting and low frequency intracellular [Ca²⁺]ᵢ resulted in activation of Ca²⁺-dependent signaling through the CnA/NFAT pathway, CnA enzyme activity was measured in skeletal muscle of 120-140 d old CON and ALS mice. Calcineurin activity increased 6.4-fold from 6.1±0.9 to 39.3±8.9 pmol/mg protein/min (p<0.05) in CON vs. ALS quadriceps muscle (FIG. 4). Consistent with this marked increase in CnA activity, higher levels of NFATc1 were observed in cytoplasmic fractions of CON mice compared to ALS mice (FIG. 5) indicating that NFAT was activated and underwent nuclear localization. Thus, increased oxidative stress in skeletal muscle combined with denervation of fast and reinervation by slow motoneurons results in increased CnA activity during muscle atrophy in ALS.

The above results showed that CnA activity was increased in the skeletal muscle of symptomatic ALS mice (i.e., 120 d-140 d) by 6.4-fold as compared to CON mice. Additionally, NFAT was activated and localized to the nucleus in ALS mice. Together, these data demonstrated that the CnA/NFAT pathway is activated during muscle atrophy in ALS mice.

### Example 4

### Alterations in Gene Expression in Skeletal Muscle of ALS Mice

Different gene expression programs are used in slow vs. fast muscle fibres and oxidative vs. glycolytic muscle fibres. Expression of TnIs, myoglobin, TnIf, MCK, and GAPDH were examined to determine if changes were occurring in the gene expression program of muscle in ALS mice.

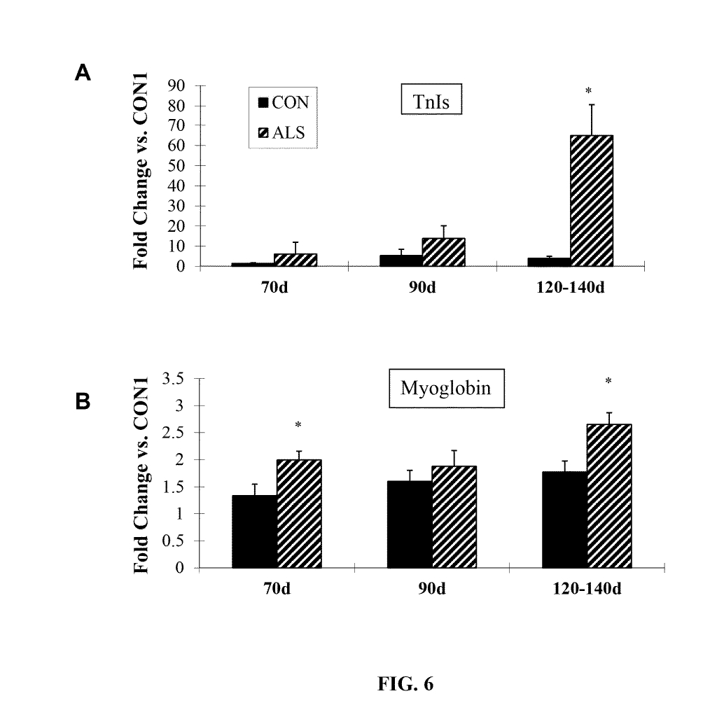

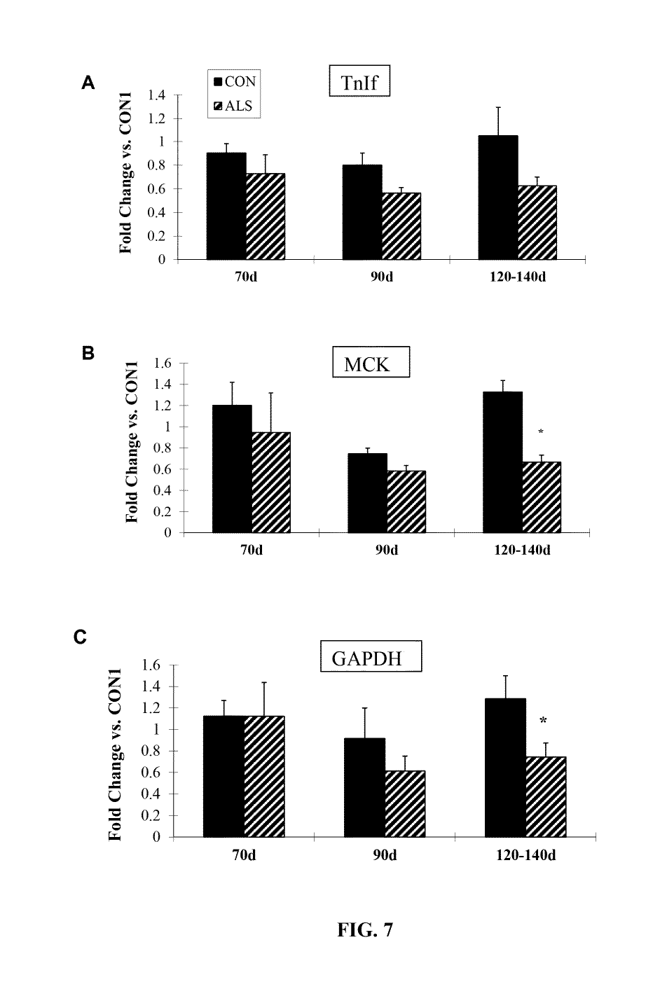

Dissection of skeletal muscles from ALS Tg mice showed that the superficial, white portion of the gastrocnemius muscles was red in appearance. Based on the change in color, the increase in resting [Ca²⁺]ᵢ and the increased CnA activity in fast muscle, increased Ca²⁺-dependent transcriptional signaling and activation of the slow fibre and oxidative gene expression programs may be occurring in the muscle of ALS mice. Accordingly, the gene expression of a set of slow/fast and oxidative/glycolytic markers in the mixed fast fibre type TA muscle was analyzed in ALS and CON mice. There was a progressive increase in the slow fibre type marker TnIs in TA muscle from the G93A*SOD1 mice, with a dramatic increase of 19-fold relative to the 120-140 d CON (p<0.05) (FIG. 6A). A significant increase in Myoglobin expression was observed in ALS mice at 70 d and 120-140 d (49 and 50% increase compared to CON, respectively; p<0.05) (FIG. 6B). There was a significant decrease in TnIf, MCK and GAPDH gene expression at 120-140 d (62%, 50% and 42% in ALS relative to CON, respectively; p<0.05) (FIG. 7). Together these data indicate an increase in the slow fibre and oxidative gene expression programs and a decrease in fast/glycolytic gene expression programs consistent with the observable shift in muscle color from white to red. The latter is consistent with activation of the slow muscle gene expression program and inhibition of the fast program by the elevations in resting Ca²⁺ and/or the elevated Ca²⁺ levels during low frequency muscle activation in ALS mice.

The above results showed significant increases in TnIs and Myoglobin gene expression in ALS mice as compared to CON mice. TnIs expression increased 19-fold and Myoglobin expression increased by about 50%. Additionally, significant decreases in TnIf, MCK, and GAPDH expression occurred in ALS mice as compared to CON mice. TnIf expression decreased by 62%, MCK expression decreased by 50%, and GAPDH expression decreased by 42%. These data indicate that the skeletal muscle in ALS mice is switching from a slow fibre and oxidative gene expression program to a fast and glycolytic gene expression program.

### Example 5

### Alterations in Skeletal Muscle Ca²⁺ Handling Proteins

Resting intracellular calcium levels in muscle are affected by reuptake of calcium, which is mediated by the SR Ca²⁺ ATPase (i.e., SERCA1 and SERCA2). Accordingly, the levels of SERCA1 and SERCA2 were examined in ALS and CON mice as discussed in more detail below.

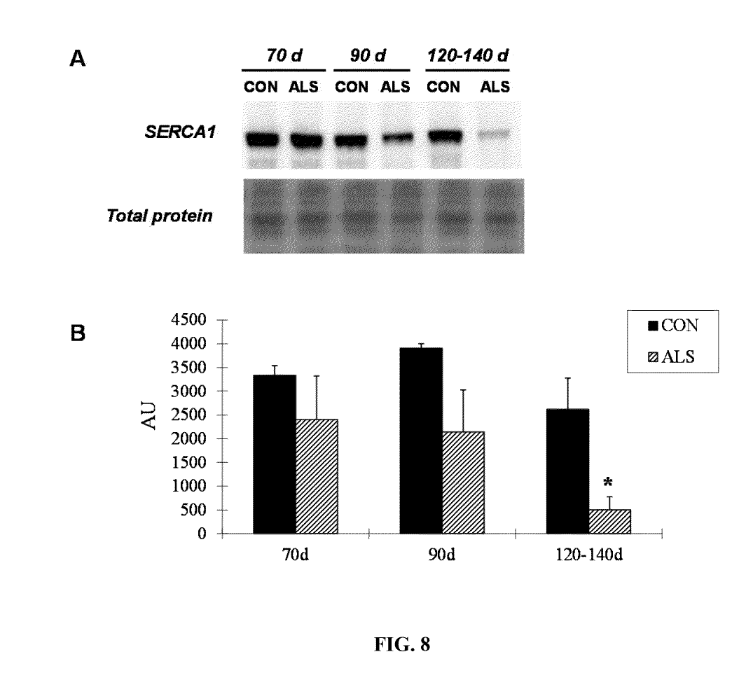

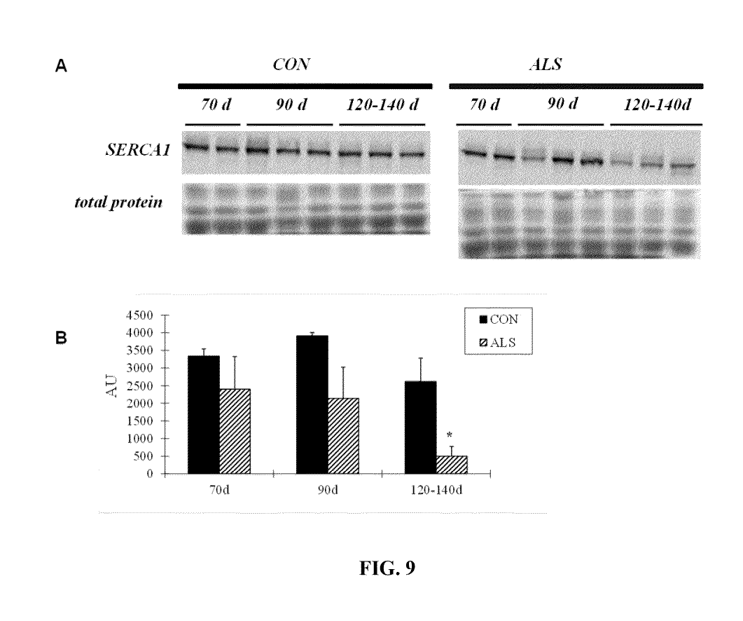

Specifically, the increase in resting intracellular Ca²⁺ and the increased time taken for [Ca²⁺]ᵢ to return to baseline after a tetanus indicated that Ca²⁺ reuptake by the SR Ca²⁺ ATPase could be impaired in muscle of ALS mice. To determine whether reduced SR Ca²⁺ pumping function was due to changes in SR protein content, protein levels of the two isoforms of SERCA (i.e., SERCA1 and SERCA2) were analyzed. SERCA1 is co-expressed with fast type II myosin heavy chain (MHC) and SERCA2 with slow type I MHC. SERCA1 protein levels were dramatically reduced in SP GAS and DP GAS muscles of ALS compared to CON mice by 120-140 d (FIG. 8, SP GAS; and FIG. 9, DP-GAS). In SP GAS from ALS mice, SERCA1 levels were reduced to 44% of CON levels (p<0.05) at 120-140 d. SERCA1 levels were not different at 70 d or 90 d, although there was considerable variability in SERCA1 levels in ALS mice at 90 d.

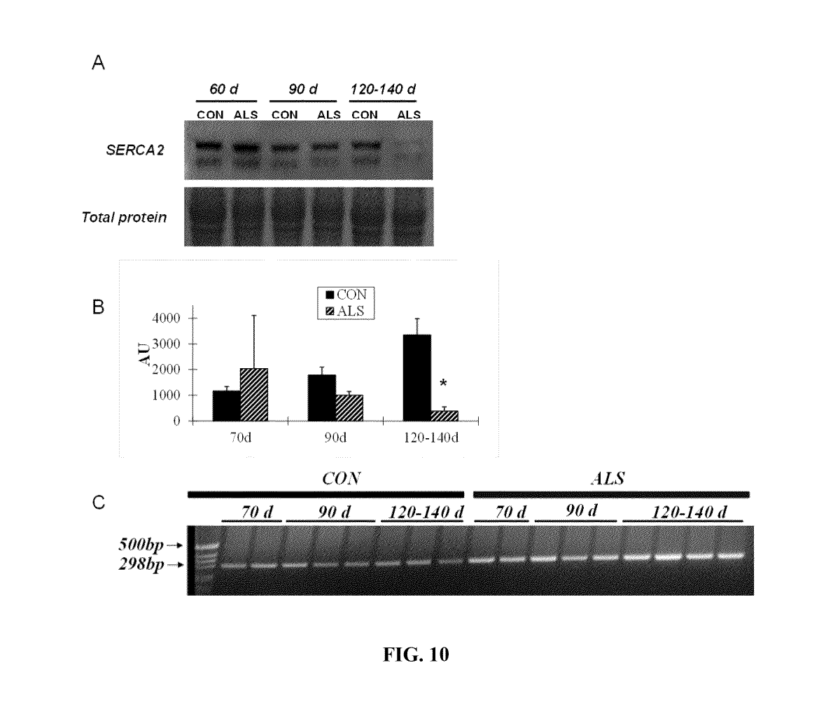

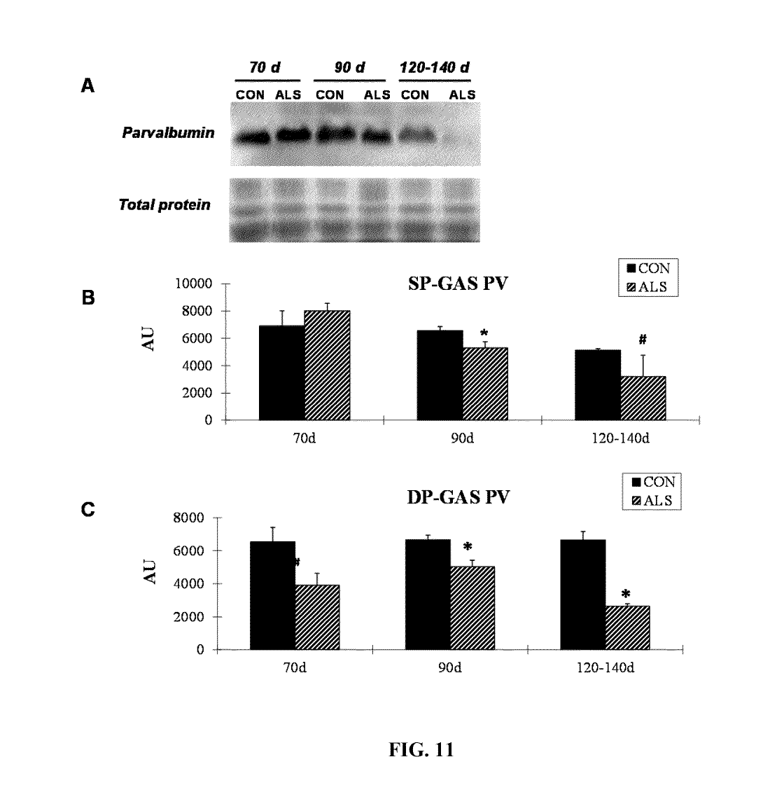

SERCA1 is the primary isoform in fast IIb fibres, and therefore, an adaptive increase in SERCA2 isoform expression in SP GAS and DP GAS muscles may occur. Accordingly, SERCA2 protein level was analyzed. Analysis, however, showed that SERCA2 was not increased but rather that it also was significantly reduced at 120-140 d, to 11% of CON levels (p<0.05) (FIGS. 10A and 10B). There was no difference in SERCA2 level in ALS mice at 70 d but at 90 d there was a tendency to be lower (59%; p=0.061). Interestingly, although there was a marked decrease in protein level, there was an increase in SERCA2 mRNA levels at 120-140 d (FIG. 10C), indicating a compensatory transcriptional adaptation for SERCA2. Overall these data showed decreased SERCA1 and SERCA2 protein levels, consistent with reduced Ca²⁺ clearance observed in single muscle fibres. These changes in SERCA level occur consistently by 120-140 d, after the increase in resting [Ca²⁺]ᵢ and the increase in low frequency peak Ca²⁺ levels, indicating that the changes in SERCA level are downstream of, and activated by the oxidative stress and the rise in intracellular Ca²⁺ levels.

The above results showed that SERCA1 and SERCA2 levels are altered in ALS mice as compared to CON mice. Specifically, SERCA1 protein levels are reduced by 44% in ALS mice. SERCA2 protein levels in ALS mice are reduced to 11% of SERCA2 levels in CON mice, however, SERCA2 mRNA levels are increased in ALS mice despite the significant decrease in SERCA2 protein levels in ALS mice. Together, these data indicate that calcium reuptake is altered in ALS mice as demonstrated by the significantly reduced levels of SERCA1 and SERCA2 protein levels in ALS mice.

### Example 6

### Parvalbumin (PV) Levels in ALS Mice

Besides calcium reuptake, cytoplasmic free calcium levels are regulated by the calcium binding proteins such as the calcium buffering protein parvalbumin (PV). PV is a calcium binding protein in the muscle, specifically, PV is expressed in a fibre-type specific manner with expression only in fast fibres (type IIb>>type IIa) and is non-existent in slow type I fibres. As discussed in more detail below, PV levels were examined in ALS and CON mice to determine if PV levels are changed in ALS mice.

In the G93A*SOD1 mice, PV protein content was significantly reduced in SP GAS at 90 d and 120-140 d to 80% and 62% of CON levels (p<0.05), respectively (FIGS. 11A and 11B). In the DP GAS, PV content was reduced as early as 70 d and persisted across all time points (61%, 75% and 40% of CON DP GAS levels at 70 d, 90 d and 120-140 d, respectively; p<0.05) (FIG. 11C). Thus, the early reduction in PV is associated temporally and coincided with the increase in resting [Ca²⁺]ᵢ.

The above results showed that PV protein levels are significantly reduced in ALS mice as compared to CON mice. Specifically, PV protein levels are reduced in pre-symptomatic (i.e., 90 d) and symptomatic (120 d-140 d) ALS mice. In the SP GAS of pre-symptomatic ALS mice, PV protein level was reduced to 80% of the level of PV protein in CON mice, while in the SP GAS of symptomatic ALS mice, PV protein level was reduced to 62% of the level of PV protein in CON mice. Furthermore, significant reductions in PV protein level were observed in DP GAS of ALS mice at 70 d, 90 d, and 120 d-140 d (i.e., 61%, 75%, and 40%, respectively). Together, these data indicated that a decrease of PV protein level occurs in ALS mice, which is detectable in pre-symptomatic ALS mice.

### Example 7

### Measurement of DHPR in ALS Mice

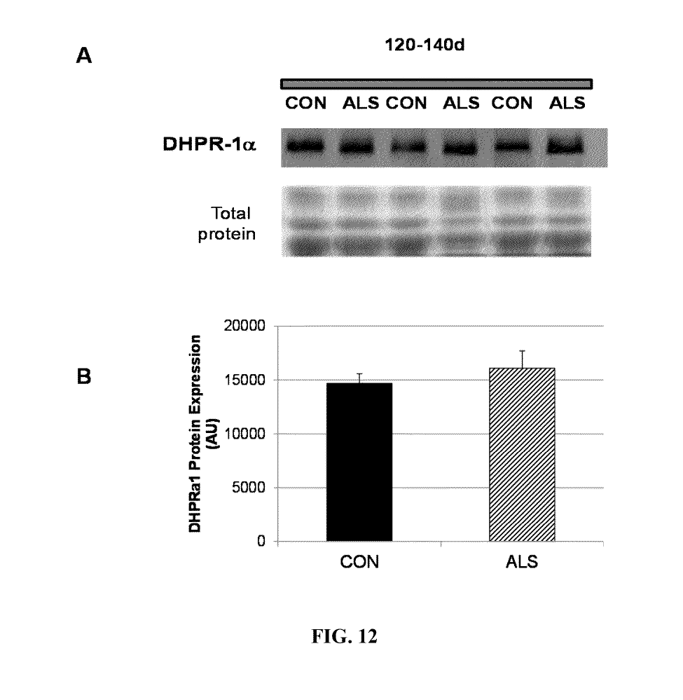

Based upon the above described changes in intracellular calcium levels at rest and during low frequency stimulatjion, the voltage sensing L-type Ca²⁺ channels (dihydropyridine receptor; DHPR) was analyzed in ALS mice (i.e., G93A*SOD1 mice) and control mice. Specifically, western blotting was utilized to examine DHPR protein levels and total protein was used as a loading control. As shown in FIG. 12, no change was observed in DHPR protein levels in ALS mice as compared to control mice, even in ALS mice that were highly symptomatic with severe muscle atrophy. DHPR, however, can be decreased in other conditions of muscle atrophy such as age-related sarcopenia. Accordingly, the above data indicated that DHPR proteins levels are unchanged in the muscle of ALS mice, even when the mice are highly symptomatic with severe muscle atrophy. Such data indicated a difference between the muscle of ALS mice suffering from muscle atrophy and other conditions in which muscle atrophy can be observed.

### Example 8

### Materials and Methods for Examples 9-11

Animals.

Control C57BL/6 SJL hybrid female and transgenic ALS B6SJL-Tg(SOD1-G93A)1Gur/J (G93A*SOD1) male mice were obtained from Jackson laboratories. Control (CON) and transgenic G93A*SOD1 heterozygote (ALS) mice were bred to establish a colony. Mice were weaned at postnatal day 21 and genotyped. Male and female ALS mice along with their wild-type littermates were investigated at a range of ages from the pre-symptomatic to the symptomatic stages of the disease: i) early pre-symptomatic at postnatal day 70 (70 d); ii) late pre-symptomatic at postnatal day 90 (90 d); and iii) symptomatic stage at postnatal days 120-140 (120-140 d) (Table 2). Early signs of disease such as muscle tremors can be detected between 65 and 90 d but overt muscle weakness and limitations in mobility do not occur until 100-120 d. At time of use, animals were euthanized by CO₂ inhalation followed by cervical dislocation. Tissues were harvested and quick frozen in liquid nitrogen for later analysis of muscle protein levels.

TABLE 2
Age and body weight of control (CON) and
transgenic G93A*SOD1 mice at time of use

| | | Age at time of use | Body weight |
|---|---|---|---|
| Age group | | (days) | (g) |
| Postnatal 70 days | CON | 69 ± 3 (n = 3) | 25.0 ± 4.9 |
| (70 d) | G93A*SOD1 | 70 ± 3 (n = 3) | 20.8 ± 3.0 |
| Postnatal 90 days | CON | 94 ± 3 (n = 5) | 21.8 ± 3.2 |
| (90 d) | G93A*SOD1 | 95 ± 3 (n = 5) | 19.3 ± 1.6 |
| Postnatal 120- | CON | 134 ± 6 (n = 3) | 29.4 ± 2.8 |
| 140 days | G93A*SOD1 | 134 ± 6 (n = 3) | 17.7 ± 1.3 |
| (120-140 d) | | | |

Protein Extraction.

The superficial gastrocnemius, diaphragm and cardiac muscle were used for assessment of proteins involved in ER stress by western blot analysis. Muscle samples were homogenized in lysis buffer (20 mM Hepes, pH 7.5, 100 mM NaCl, 1.5 mM MgCl2, 0.1% Triton X-100, 20% Glycerol) containing 1 mM DTT and protease inhibitor cocktail (Complete mini EDTA-free Protease Inhibitor Cocktail, Roche). After 20 min of incubation at 4° C. followed by centrifugation for 5 min at 20,000×g, the supernatant was collected and frozen at −80° C. until required.

Immunoblotting.

Total protein was determined using a BCA protein assay kit (Thermo Scientific). Samples were then solubilized in loading buffer and denatured (5 min at 100° C.). For assessment of changes in protein expression levels, 15-30 μg total protein were loaded on bis-acrylamide gels and analyzed by polyacrylamide gel electrophoresis (PAGE). The amount of protein loaded for each endpoint was determined by separate analysis of the linear range for each antibody. Samples were then transferred to PVDF membrane (Millipore) and blocked with 5% (w/v) non-fat dry milk powder in Tris-buffered saline (pH 8.0) for 1 hr. The appropriate primary antibodies were added (PERK, IRE1α, PDI, CHOP, and caspase-12; 1:1000, Cell Signaling Technology) and membranes were incubated for 1 hr at room temperature and subsequently probed with HRP-linked anti-rabbit IgG or anti-mouse IgG (1:1000, Cell Signaling Technology) 1 hr at room temperature. Secondary antibodies were detected using HRP-linked chemiluminescence with SuperSignal West Dura Chemiluminescence Substrate (Thermo Scientific) and imaged using a chemiluminescence imaging system (GeneGnome, Syngene). The signal for the target protein of each sample was quantified and expressed in arbitrary unit (AU) and then values for each ALS and CON mouse expressed as a ratio relative to the first CON mouse (CON1) at each age. Images for total proteins are shown to confirm equal loading across samples.

Data Analysis.

Data were analyzed using a one-way ANOVA. Tukey's post hoc analysis were used for all significant ANOVAs and p<0.05 used to determine statistical significance.

### Example 9

### ER Stress Pathway is Induced in Skeletal Muscle of ALS Mice by 70 d

PERK and IRE1α are involved in sensing ER stress and are upregulated when ER stress is induced. PDI is an ER chaperone that is induced during ER stress. Accordingly, PERK, IRE1α, and PDI protein levels were analyzed to determine if the ER stress pathway is induced in the skeletal muscle of ALS mice relative to CON mice.

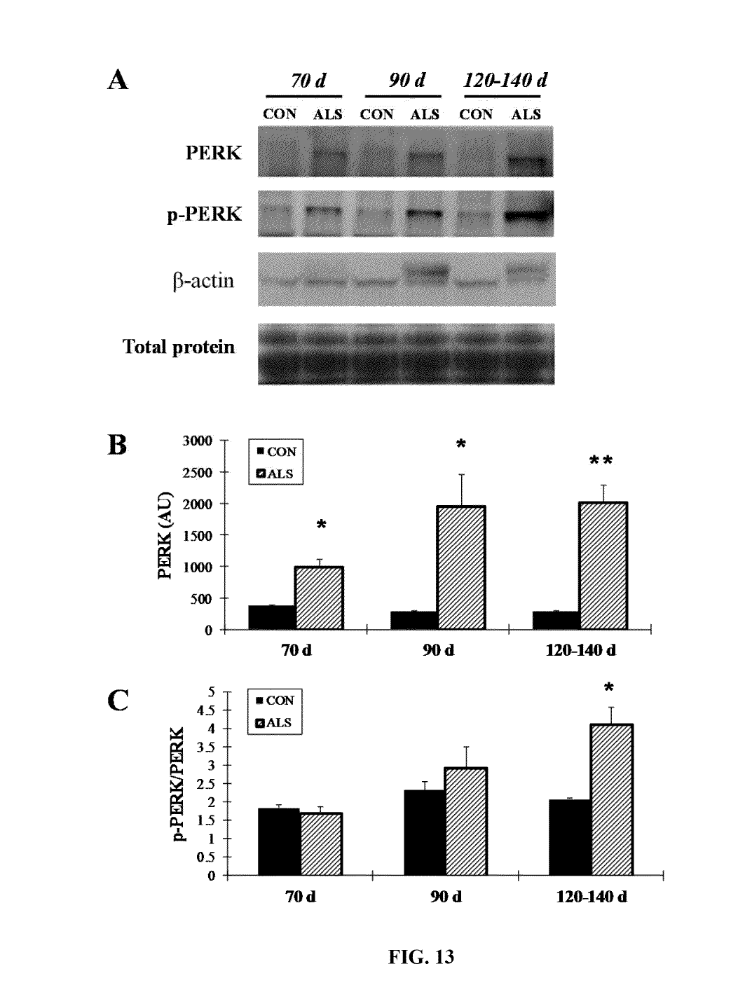

Specifically, up-regulation of PERK was observed in superficial gastrocnemius muscle of ALS mice at 70 d (2.7±0.2-fold), 90 d (5.4±0.6-fold), and 120-140 d (5.2±0.9-fold, p<0.05; FIG. 13). Up-regulation of phosphorylated PERK (i.e., p-PERK) was also observed in ALS mice (FIGS. 13A and 13C).

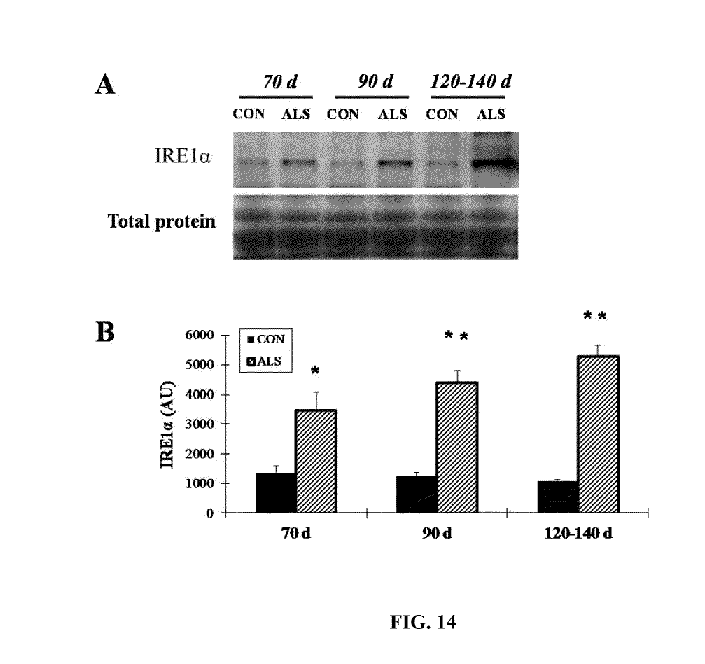

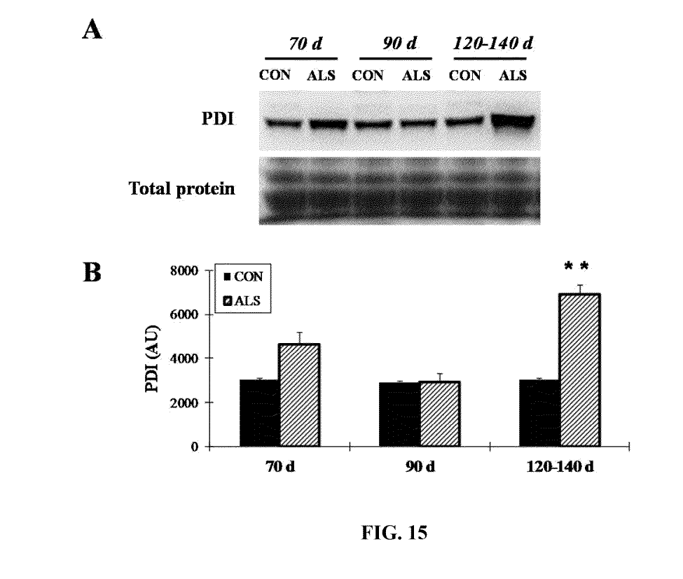

Up-regulation of IRE1α was also observed at 70 d (2.5±0.2-fold, p<0.05), 90 d (3.5±0.1-fold, p<0.01) and 120-140 d (4.9±0.1-fold, p<0.01) in superficial gastrocnemius muscle (FIG. 14). Furthermore, ER chaperone PDI was significantly up-regulated at 120-140 d (2.3±0.1-fold, p<0.01; FIG. 15), in superficial gastrocnemius muscle (p=0.1).

The above results showed that in superficial gastrocnemius muscle of ALS mice, ER stress was initiated at an early or pre-symptomatic stage of ALS because of the significantly higher levels of PERK, IRE1α, and PDI at 70 d and/or 90 d in ALS mice as compared to CON mice. The above results also showed that ER stress continued into the symptomatic stage (i.e., 120 d-140 d) stage of ALS because of the significantly higher levels of PERK, IRE1α, and PDI at in ALS mice as compared to CON mice. These data indicated that ER stress sensors and ER chaperone proteins are up regulated in both the pre-symptomatic (i.e., 70 d and 90 d) and symptomatic (i.e., 120-140 d) stages of ALS.

### Example 10

### ER Stress Specific Cell Death Signals are Induced in Skeletal Muscle but not Cardiac Muscle of ALS Mice

ER stress can lead to cell death via the activation of CHOP and caspase-12. Caspase-12 is activated by apoptotic signals including an ER stress component, but not by those apoptotic signals that do not induce ER stress. Caspase activation, including caspase-12 activation, is detected by cleavage of the caspase into smaller molecular weight subunits. Additionally, atrophy of the diaphragm muscle can result in respiratory failure and death in ALS mice. As such, the levels of CHOP protein and caspase-12 cleavage were examined in the skeletal, diaphragm, and cardiac muscle of ALS mice.

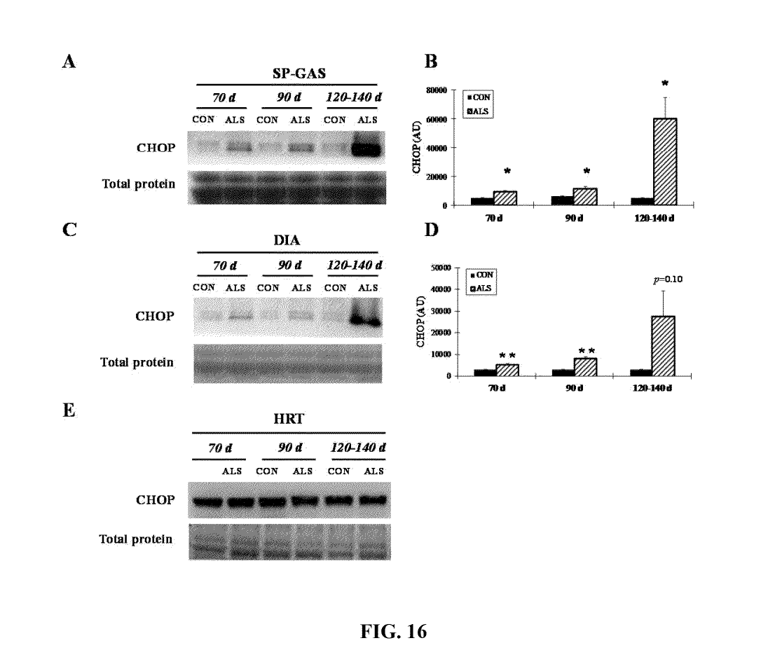

Specifically, quantitative Western blot analyses revealed that CHOP was up-regulated in superficial gastrocnemius at 70 d (1.9±0.1-fold, p<0.05), 90 d (2.4±0.2-fold, p<0.05), and quite dramatically at 120-140 d (13.3±1.7-fold, p<0.05, FIGS. 16A and 16B). CHOP was also up-regulated in diaphragm muscle (FIGS. 16C and 16D), but not in the cardiac muscle (FIG. 16E) of assessed ALS mice.

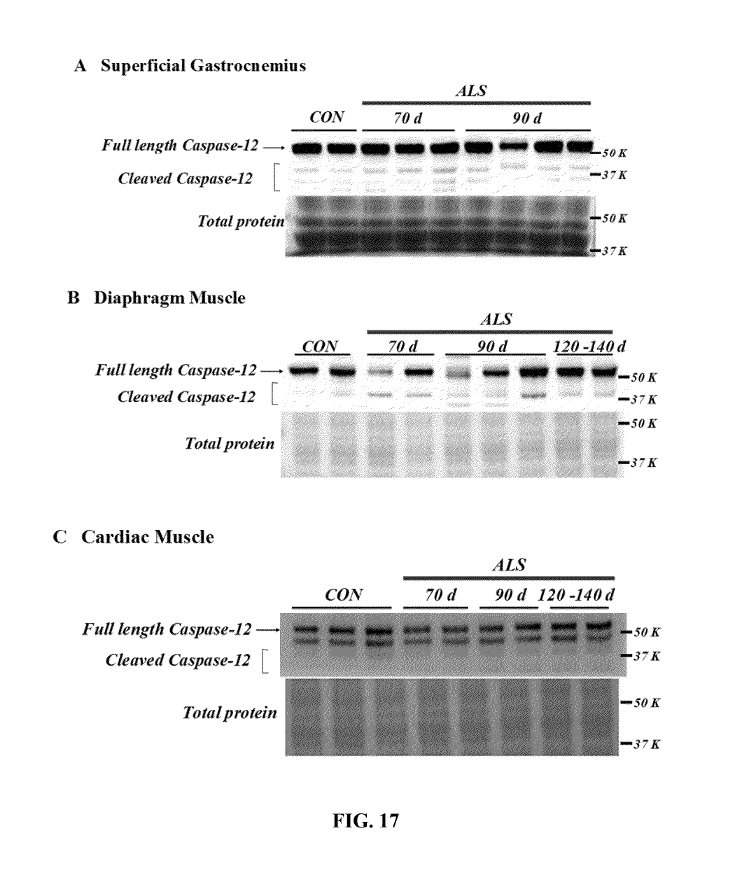

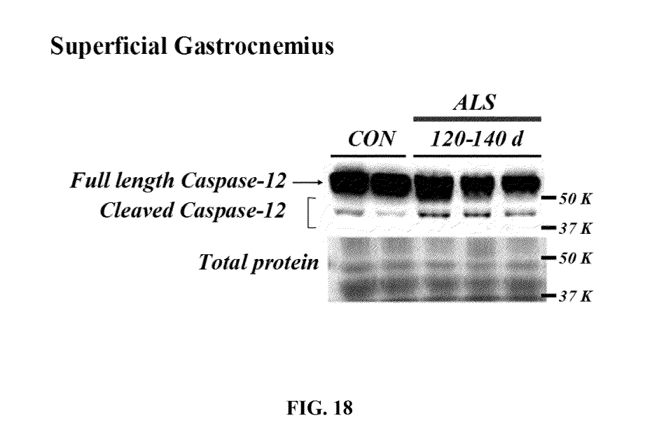

Western blot analyses also revealed that caspase-12 was cleaved more extensively in superficial gastrocnemius muscle of ALS compared to CON mice at all disease stages (FIGS. 17A and 18), indicating activation of apoptosis in skeletal muscle. Furthermore, increased levels of CHOP protein and cleaved caspase-12 were observed in the diaphragm muscle of ALS mice at all disease stages (FIG. 17B). However, in cardiac muscle, CHOP and cleaved caspase-12 levels did not differ between ALS mice and age-matched CON mice (FIGS. 16E and 17C). These data indicated that apoptosis is upregulated in a skeletal and diaphragm muscle-specific manner.

The above results indicated that CHOP protein levels are significantly increased in the pre-symptomatic (i.e., 70 d and 90 d) and symptomatic (i.e., 120-140 d) stages of ALS in both the skeletal and diaphragm muscles of ALS mice. The above results also indicated that caspase-12 cleavage was significantly increased in the pre-symptomatic (i.e., 70 d and 90 d) and symptomatic (i.e., 120-140 d) stages of ALS in both the skeletal and diaphragm muscles of ALS mice. CHOP protein levels and capase-12 cleavage were unaltered in the cardiac muscle of ALS mice at both pre-symptomatic and symptomatic stages of ALS. Together, these data showed that apoptosis is activated or upregulated in both skeletal and diaphragm muscle of ALS mice. Furthermore, such upregulation of apoptosis occurred at both the pre-symptomatic and symptomatic stages of ALS.

### Example 11

### Measurement of Factors Involved in Protein Synthesis in ALS Mice

As shown above, ER stress is induced in ALS mice, leading to apoptosis. Another consequence of ER stress can be a decrease in or a down-regulation of protein synthesis. Phosphorylated eIF2α (i.e., p-eIF2α), phosphorylated p70S6K (i.e., phospho p70S6K), and phosphorylated Akt (i.e., phospho Akt) protein levels can be a read out of the amount of protein synthesis occurring in a cell. Elevated levels of p-eIF2α can be correlated with a decrease or a down-regulation of protein synthesis while elevated levels of phospho p70S6K and phosphor Akt can be correlated with an increase or an up-regulation of protein synthesis. Accordingly, the levels of p-eIF2α, phospho p70S6K, and phospho Akt were analyzed in ALS mice (i.e., G93A*SOD1 mice) via western blotting.

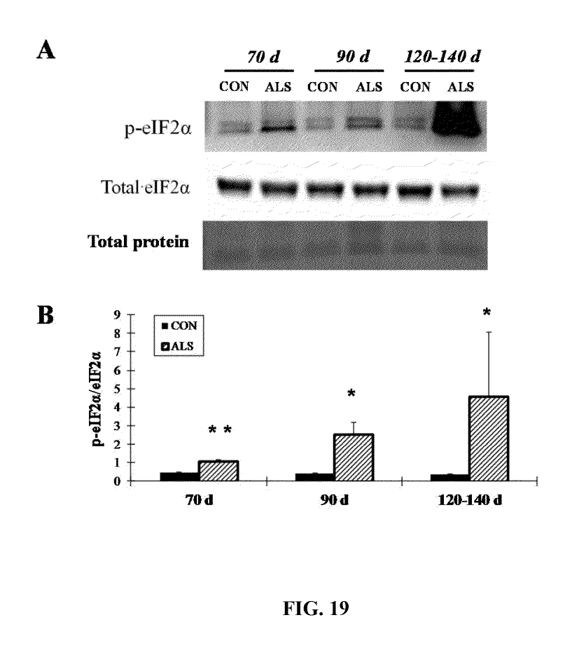

FIG. 19 showed that p-eIF2α protein levels were increased, elevated, or raised at 70 d, 90 d, and 120-140 d in ALS mice as compared to control mice. Such data indicated that p-eIF2α protein levels are elevated, and thus protein synthesis is down-regulated, at both the pre-symptomatic and symptomatic stages of ALS in the mice.

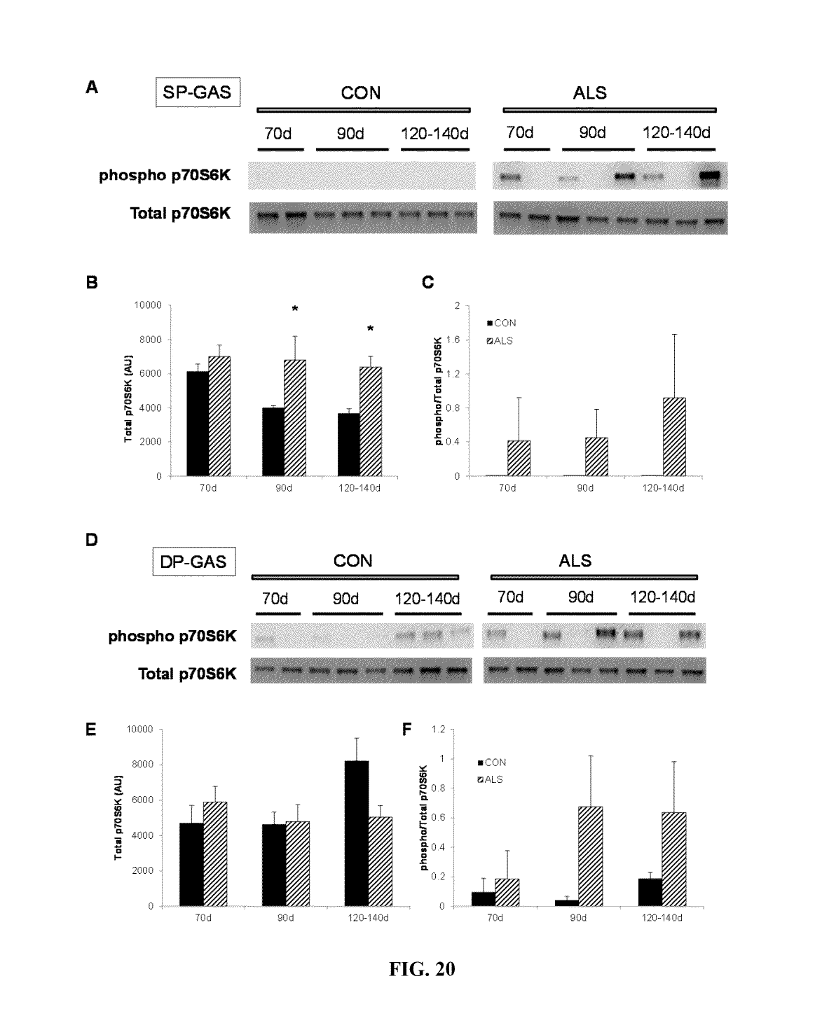

FIG. 20 showed that total p70S6K protein levels were elevated at 90 d and 120-140 d in ALS mice as compared to control mice in SP-GAS muscle. Total p70S6K protein levels, however, were not elevated in DP-GAS muscle of ALS mice as compared to control mice (FIGS. 20D and 20E). Phospho p70S6K was detected in ALS mice in both SP-GAS and DP-GAS muscle, and the ratio of phospho p70S6K:total p70S6K indicated a trend towards increased phosphorylation of the pool of p70S6K in SP-GAS and DP-GAS muscle of ALS mice (FIG. 20).

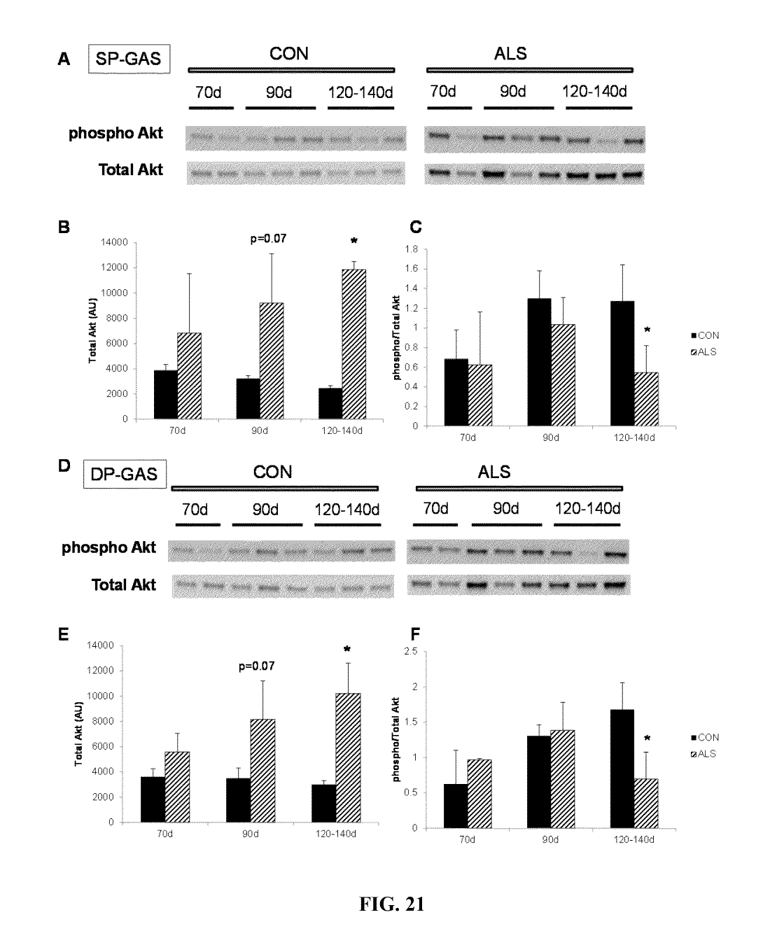

FIG. 21 showed that total Akt protein levels were elevated in the SP-GAS and DP-GAS of ALS mice as compared to control mice, particularly at 120-140 d. Phospho Akt protein levels in SP-GAS and DP-GAS of ALS mice, however, were unchanged as compared to control mice when expressed as a ratio of phospho Akt:total AKT, except at 120-140 d when the ratio of phospho Akt:total Akt was significantly decreased in SP-GAS and DP-GAS of ALS mice as compared to control mice.

The above data indicated that protein synthesis can be down-regulated or decreased in ALS mice given the increase in p-eIF2α levels at both pre-symptomatic and symptomatic stages of ALS. Total p70S6K and total Akt levels were elevated in the skeletal muscle of ALS mice at symptomatic stage of ALS (i.e., 120-140 d). The ratio of phospho Akt:total Akt was significantly decreased at the symptomatic stage of ALS (i.e., 120-140 d), thereby indicating that protein synthesis can be down-regulated in the skeletal muscle of ALS mice experiencing symptoms of ALS.

### Example 12

### Proteomic Analysis of ALS Skeletal Muscle

Materials and Methods.

Briefly, muscle lysates were digested with trypsin then peptide fragments were labeled with isobaric tags using the iTRAQ system (Applied Biosystems) and analyzed by nano Liquid Chromatography tandem Mass Spectrometry (nanoLC MSMS) (60). This tandem mass tag labeling systems provides relative quantitation of peptide abundance between samples. One quadriceps muscles was analyzed from 6, 18 and 30 mos. old mice along with a soleus muscle (slow oxidative) muscle from a 6 mos. old mouse. The International Protein Index (IPI) mouse database was used to analyze the data with Mascot and Sequest search engines. Search engines look for modified peptides and compare ratios of the reporting groups to determine the relative quantity of the peptides. Identified proteins are reported as—fold increase relative to the 6 mos. old quadriceps muscle. Data shown in Table 1 include all proteins identified that increased>1.5-fold or decreased>50% or were of interest based on their cellular function.

Quadriceps muscles were used for proteomic analysis. Samples were homogenized, and supernatants obtained by centrifugation and then concentrated with 10 kDa mw cut off spin cartridge. The concentrated samples were recovered, reduced, alkylated and digested with trypsin overnight. Tryptic digests were then desalted and labeled using isobaric mass tags. For quantitative comparisons between ALS and CON mice and across the lifespan in control mice (6, 12 and 18 mos.), tryptic peptides were labeled after digestion using the iTRAQ system. The tryptic digests will react with reagents where mass tags will covalently bind to the amino termini lysine residues. All samples were treated with different tags to allow simultaneous determination of both identity and relative abundances of peptide pairs across the samples. Labeled peptides were transferred to autosampler for analysis by nanoLC MS/MS. The same peptide from different samples elute from HPLC at the same time with the same mass. Search engines identified the modified peptides and compared ratios of the reporting groups to determine the relative quantity of the peptides across samples.

The International Protein Index (IPI) mouse database was used to analyze the data with Mascot and Sequest search engines. Search results were merged for protein identification with Scaffold Distiller. Multiple peptides from the same protein were used to determine the relative quantity of the samples. Proteomics data were triaged based on the following criteria: i) >2 peptides used to identify the protein; ii) relative change in protein is >50% (increase or decrease); iii) proteins of interest based on cellular function were analyzed.

Results.

These proteins were identified by isotope labeling methods combined with liquid chromatography and tandem mass spectroscopy (LC-MS/MS). Quadriceps muscle lysates for 70 d old CON and ALS mice and 6, 18 and 30 mos. old C57BL/6 (Control) mice were analyzed using an iTRAQ 8-plex. Using this proteomics approach, 3293 proteins were identified. The relative abundance of each protein, was determined by spectral count and then the ratio of these quantified spectra were used to calculate a ratio for the change in ALS compared to CON muscle (ALS/CON) for each protein. Table 1 shows the proteins that were either increased by >1.5-fold or decreased by more than 50% (i.e. 0.5-fold or less). The patterns identified for changes in ALS vs. CON proteins were: 1) increased in ALS vs. CON; 2) decreased in ALS vs. CON; 3) present/identified in CON but not in ALS (i.e. proteins whose peptide fragments were below detection limits in ALS samples). To understand ALS-specific muscle atrophy, these proteins were also compared to changes in protein abundance in muscle from 30 mos. vs. 6 mos. old mice (i.e. mice with age-related atrophy or sarcopenia). The proteins which were altered in ALS but not with sarcopenia, represent ALS-disease specific changes (ALS/Aging). The ALS-specific atrophy proteins identified include 1 that is upregulated and 12 that are decreased or not-detectable.

TABLE 3
Proteins Altered in ALS mice.

| | MW | | | ALS/ | | 30 mos. | ALS/ |
|---|---|---|---|---|---|---|---|
| Description | [kDa] | CON | ALS | CON | Pattern | vs 6 mos. | Aging |
| Fructose-bisphosphate aldolase A | 39.4 | 0.821 | 1.690 | 2.058 | 1 | 0.719 | 2.862 |
| Isoform 3 of Coiled-coil domain- | 46.0 | 1.420 | 2.895 | 2.039 | 1 | 1.985 | 1.028 |
| containing protein 91 | | | | | | | |
| Fatty acid-binding protein, epidermal | 15.2 | 0.725 | 1.436 | 1.981 | 1 | | |
| cDNA FLJ54108, highly similar to Homo sapiens | 36.3 | 0.800 | 1.543 | 1.929 | 1 | 2.033 | 0.949 |
| smooth muscle cell associated | | | | | | | |
| Isoform 2 of Ankyrin repeat domain- | 36.2 | 1.422 | 0.715 | 0.503 | 2 | 0.193 | 2.607 |
| containing protein 2 | | | | | | | |
| Isoform 2 of Regucalcin | 25.0 | 1.176 | 0.563 | 0.479 | 2 | 0.677 | 0.708 |
| cDNA FLJ54106, moderately similar to Synaptic | 34.9 | 0.748 | 0.318 | 0.425 | 2 | 0.989 | 0.430 |
| vesicle membrane protein VAT-1 homolog | | | | | | | |
| Phosphorylase | 87.3 | 0.630 | 0.265 | 0.420 | 2 | 0.822 | 0.511 |
| BTB/POZ domain-containing protein KCTD11 | 25.9 | 0.685 | 0.282 | 0.411 | 2 | 1.492 | 0.275 |
| Leucine-rich repeat-containing protein 14 | 54.5 | 1.083 | 0.341 | 0.315 | 2 | 0.937 | 0.336 |
| Isoform 1 of Transmembrane protein 132D | 122.2 | 1.061 | 0.327 | 0.308 | 2 | 0.897 | 0.344 |
| Prothrombin (Fragment) | 70.0 | 0.998 | 0.304 | 0.305 | 2 | 0.949 | 0.321 |
| cDNA FLJ53099, highly similar to Beta-enolase | 29.5 | 1.084 | 0.299 | 0.276 | 2 | 0.947 | 0.291 |
| Creatine kinase M-type | 43.1 | 1.051 | 0.288 | 0.274 | 2 | 0.834 | 0.328 |
| Creatine kinase, sarcomeric mitochondrial | 47.5 | 1.140 | 0.285 | 0.250 | 2 | 0.463 | 0.540 |
| Isoform 1 of Coiled-coil domain-containing | 86.7 | 0.637 | 0.144 | 0.227 | 2 | 1.613 | 0.141 |
| protein C6orf199 | | | | | | | |
| Cytochrome c oxidase subunit 4 isoform 1, | 19.6 | 1.150 | 0.215 | 0.187 | 2 | 1.090 | 0.171 |
| mitochondrial | | | | | | | |
| Similar to ATPase, Ca++ transporting, | 92.3 | 0.476 | | 0.000 | 4 | 1.456 | 0.000 |
| cardiac muscle, fast twitch 1 (Fragment) | | | | | | | |
| ACTA2 protein (Fragment) | 36.8 | 0.655 | | 0.000 | 4 | 2.896 | 0.000 |
| protein tyrosine phosphatase, receptor type, | 212.4 | 0.979 | | 0.000 | 4 | 0.877 | 0.000 |
| sigma isoform 2 precursor | | | | | | | |
| L-lactate dehydrogenase | 26.7 | 0.757 | | 0.000 | 4 | 0.888 | 0.000 |
| Putative uncharacterized protein GPKOW | 39.7 | 0.598 | | 0.000 | 4 | 0.961 | 0.000 |
| L-lactate dehydrogenase | 25.2 | 0.484 | | 0.000 | 4 | 0.879 | 0.000 |

Summary of Results.

Proteins increased or upregulated in ALS mice included fructose-bisphosphatase Aldolase A. Proteins that were decreased or downregulated in ALS mice included: a cDNA FLJ53099, highly similar to Beta-enolase, muscle-type creatine kinase, mitochondrial CK and mitochondrial cytochrome oxidase subunit 4 (proteins regulating metabolism) and isoform 1 of coiled-coil domain protein C6orf199. Also notable was the decrease to undetectable levels of SR Ca²⁺ ATPase, ACTA2, a protein phosphatase receptor and LDH in the ALS muscle. Overall, the shift in muscle proteome in pre-symptomatic 9 wks old ALS mice indicated a decrease in mitochondrial proteins, glycolytic enzymes, SR Ca²⁺ regulatory protein as well as structural and transcriptional regulators.

### Example 13

### Materials and Methods for Examples 15-18

Study Parameters.

The study used 12 mice. At 35 d, mice were assigned to treatment groups. The 3 groups in the study were: i) wild-type control mice treated with vehicle (CON-Veh; n=4; 3 female (F) and 1 male (M)); ii) G93A*SOD1 ALS mice treated with vehicle (ALS-Veh; n=4; 3 F and 1 M; and iii) G93A*SOD1 mice treated with 6-Gingerol (ALS-Gin; n=4; 3 F and 1 M). There were no 6-gingerol treated control mice due to cost of the drug.

Drug Treatment.

6-gingerol was purchased from Carbosynth Limited, UK. Mice were dosed daily with intraperitoneal (ip) injection of vehicle (0.4% ethanol in PBS) or 6-gingerol at a dose of 10 mg/kg. Treatment began when mice were 35 d old and terminated at 115 d. The volume dosed was adjusted weekly according to changes in body weight.

Grip Function Test.

Briefly, mice are placed on a metal grid (20 cm wide, 40 cm long; grid placed 40 cm above table) and allowed to grip with fore- and hind-limb paws prior to inverting the grid. Timing begins once the grid is inverted and stops once the mouse can no longer hold the lid.

Stride Length Test.

Briefly, fore- and hind-limb paws are dipped in non-toxic paint and mice walk across a 120 cm table covered in white paper. A dark box with food is placed at the far end to encourage walking across the table. The distance between the fore- and hind-limb ink marks are then measured. Four strides per mouse are quantified and the average stride length determined

Measurement of Intracellular Calcium Handling.

After conclusion of the grip function and stride function tests, mice were then sacrificed and single muscle fibres were obtained immediately from the flexor digitorum brevis (FDB) muscle and intracellular Ca²⁺ handling assessed using Fura-2 as discussed above in Example 1. Changes in Fura-2 ratio, representing changes in [Ca²⁺]i, were measured under resting conditions and in response to electrical stimulation at a range of physiological frequencies (10-150 Hz).

Measurement of Muscle Protein Content.

After conclusion of the grip function and stride function tests, mice were then sacrificed and skeletal muscle tissue was quick frozen in liquid nitrogen for subsequent analyses of muscle protein content. SERCA1 and SERCA 2 protein levels were analyzed by western blotting as described in Example 1.

### Example 14

### 6-Gingerol Improves Muscle Function in ALS Mice

Ginger has anti-inflammatory effects and one of the components of ginger is the compound 6-gingerol. 6-gingerol has anti-oxidant, anti-apoptotic, and anti-inflammatory properties. As discussed above, SERCA (i.e., SERCA1 and SERCA2) protein levels are significantly decreased in ALS mice, resulting in increased resting [Ca²⁺]ᵢ levels. SERCA has cysteine residues involved in the calcium binding and transport function of SERCA. Cysteine residues, however, can be affected by reduction/oxidation (redox) mechanisms, and as such, redox mechanisms could alter SERCA activity. Accordingly, 6-gingerol was administered to ALS mice to determine if 6-gingerol could alter SERCA activity and therefore, disease progression in ALS mice.

Specifically, muscle function was assessed using a grip test and a walking or stride test at the termination of the study. After conclusion of the grip function and stride function tests, the mice were then sacrificed and skeletal muscle tissues weighed to determine muscle mass.

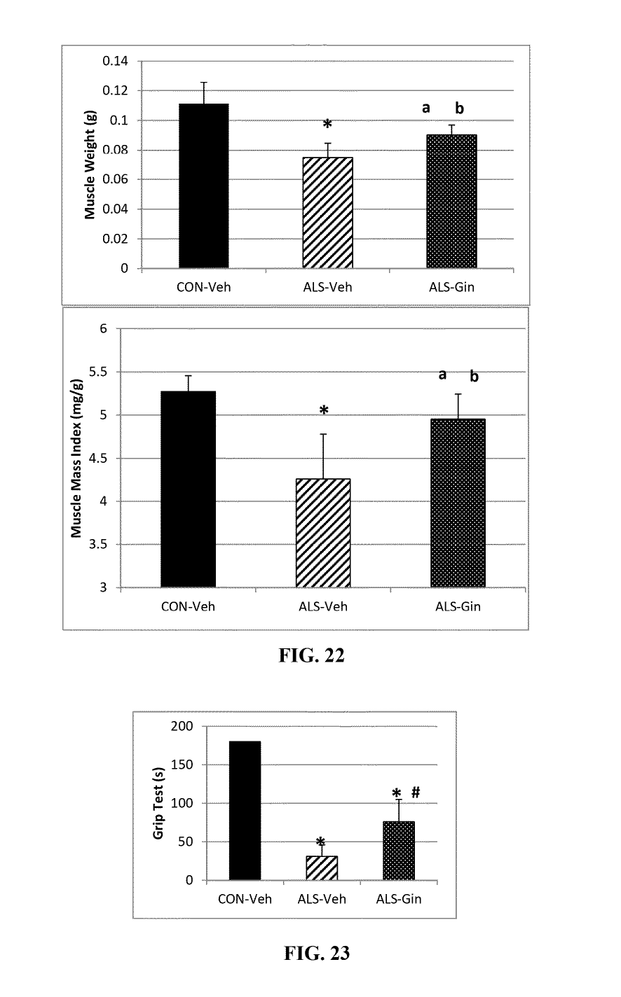

G93A*SOD1 ALS mice begin to exhibit early signs of motor dysfunction at ˜75 d and symptoms progress to paralysis by ˜125 d. In this study, mice were evaluated at an average age of 115±5 d (for CON-Veh, ALS-Veh and ALS-Gin), just prior to severe symptom onset. There was a significant reduction in body weight in ALS compared to CON mice, but no significant improvement with 6-gingerol treatment (CON-Veh: 21.0±2.7 g; ALS-Veh: 17.8±1.4 g; ALS-Gin 18.2±0.7 g) (Table 4). There was also a significant reduction in muscle mass in ALS vs. CON mice and a 21% improvement in muscle mass with 6-gingerol (CON-Veh: 0.111±0.015 g; ALS-Veh: 0.075±0.010 g; ALS-Gin 0.090±0.007 g) (FIG. 22, left panel, and Table 4). The muscle mass index (mg muscle per g body weight) was also reduced in ALS-Veh vs. CON-Veh and attenuated in ALS-Gin (FIG. 22, right panel, and Table 3).

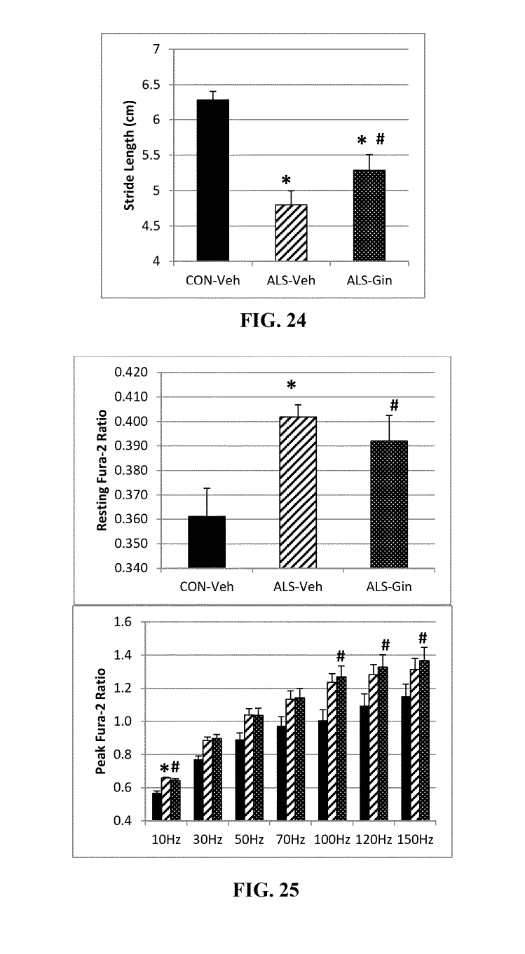

Skeletal muscle function was assessed using a grip test where the amount of time that mice could grip a wire grid was measured. Grip function was reduced in ALS-Veh to 17% of CON-Veh level (p<0.05) and showed a tendency to improve (to 42% of CON-Veh; p=0.08) with 6-gingerol treatment (FIG. 23 and Table 4). Mobility of the mice was assessed using a walking test where paws were marked with ink, mice walked across a flat surface and stride length was assessed by the distance between paw marks. Stride length was shorter in ALS-Veh compared to CON-Veh (*p<0.05) and showed a tendency to improve in ALS-Gin compared to ALS-Veh (p=0.13) (FIG. 24 and Table 4).

TABLE 4
Summary of changes is G93A*SOD1
mice with 6-gingerol treatment.

| | Δ in ALS-Veh* | Δ in ALS-Gin* | Effect of 6-gingerol |
|---|---|---|---|
| Body weight | ↓ 16% | ↓ 13% | no improvement |
| Gastrocnemius | ↓ 33% | ↓ 19% | # p = 0.09 |
| weight (mg) | | | |
| Gastrocnemius | ↓ 19% | ↓ 6% | # p = 0.11 |
| weight (mg/g) | | | |
| Grip test (s) | ↓ 83% | ↓ 58% | p = 0.08 |
| Stride length (cm) | ↓ 24% | ↓ 16% | # p = 0.13 |

*Percent change (Δ) is expressed relative to CON-Veh.

The above results showed that ALS mice administered 6-gingerol did not have an improved or increased body weight as compared to ALS mice administered the vehicle alone. The above results, however, showed that ALS mice administered 6-gingerol did have a 21% improvement in muscle mass. Additionally, skeletal muscle function was improved in ALS mice administered 6-gingerol as evidenced by improved grip function and stride function.

### Example 15

### 6-Ginerol Improves Intracellular Calcium Clearance in ALS Mice

As discussed above, 6-gingerol has anti-oxidant, anti-apoptotic, and anti-inflammatory properties and administration of 6-gingerol improved muscle function in ALS mice. As discussed in Example 2, ALS mice have increased resting [Ca²⁺]ᵢ levels. Accordingly, the mice of Example 11 were examined to determine if 6-gingerol administration improves intracellular calcium handling in ALS mice.

Specifically, changes in intracellular Ca²⁺ handling were measured in skeletal muscle using isolated single muscle fibres. Consistent with Example 2, resting Fura-2 ratio was significantly increased in ALS-Veh vs. CON-Veh. An increase in Fura-2 ratio with low (10 Hz) but not high frequency stimulation was also observed. A significant increase in the Fura-2 ratio in ALS-Veh compared to CON-Veh at 10 Hz was observed, consistent with Example 2.

The Fura-2 ratio was lower but did not reach statistical significance (p=0.13) in ALS-Gin vs. ALS-Veh. There was also a tendency for the Fura-2 ratio to be increased at 10 Hz and higher stimulation frequencies in fibres from ALS-Gin mice. Interestingly, there was no attenuation of the increase in peak Fura-2 ratio with 6-gingerol treatment, indicating that there would still be a Ca²⁺ overload during muscle activation in vivo (FIG. 25).

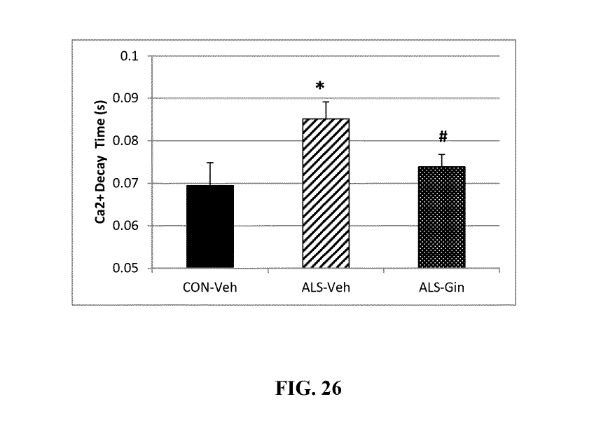

In order to assess SR Ca²⁺ pump function, intracellular Ca²⁺ clearance following stimulation was determined by the time taken for Fura-2 ratio to return to 25% of its baseline level. This Ca²⁺ decay time was measured following 50 and 100 Hz tetani. There was a significant increase in Ca²⁺ decay time in ALS-Veh compared to CON-Veh and there was a tendency for improvement in ALS-Gin vs. ALS-Veh (FIG. 26).

TABLE 5
Summary of changes in G93A*SOD1
mice with 6-gingerol treatment

| | Δ in ALS-Veh* | Δ in ALS-Gin* | Effect of 6-gingerol |
|---|---|---|---|
| Resting Fura-2 | ↑ 11% | ↑ 8.5% | # p = 0.13 |
| ratio | | | |
| Peak Fura-2 ratio | ↑ 16% | ↑ 13% | no improvement |
| (10 Hz) | | | |
| Ca²⁺ decay | ↓ 22% | ↓ 6% | # p = 0.11 |

*Percent change (Δ) is expressed relative to CON-Veh.

The above data showed that 6-gingerol treatment improved intracellular calcium clearance after stimulation even though the peak Fura-2 ratio remained elevated in ALS mice receiving 6-gingerol treatment. The above data also showed that 6-gingerol treatment of ALS mice reduced the increase in resting [Ca²⁺]ᵢ observed in ALS mice.

### Example 16

### SERCA1 Protein Levels Increased in the Skeletal Muscle of ALS Mice Administered 6-Gingerol

As shown in Example 5, SERCA1 and SERCA2 protein levels are decreased in ALS mice, thereby causing decreased or reduced intracellular calcium clearance in ALS mice. 6-gingerol treatment, however, improved intracellular calcium clearance in ALS mice as discussed above. Accordingly, SERCA1 protein levels were examined in ALS mice receiving 6-gingerol treatment.

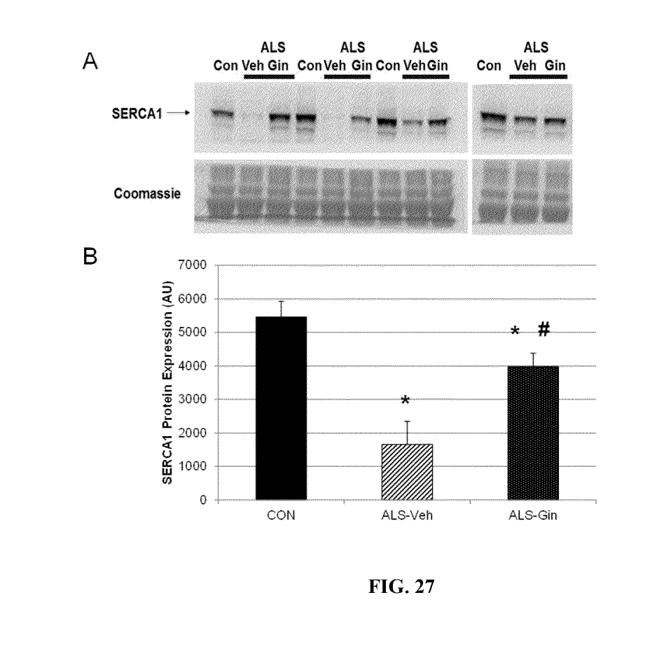

A dramatic increase in SERCA1 was observed in gastrocnemius muscle of ALS-Gin compared to ALS-Veh with protein levels nearing those found in CON muscle (FIG. 27). There was minimal rescue of the decrease in SERCA2 with 6-gingerol treatment.

The above results showed that 6-gingerol treatment of ALS mice restored SERCA1 protein levels, thereby allowing for reuptake of intracellular calcium and improved clearance of intracellular calcium.

### Example 17

### CHOP Protein Levels are Decreased in ALS Mice Administered 6-Gingerol

The response of skeletal muscle to the ER stress markers was also assessed in response to gingerol treatment, namely by examining the levels of the apoptotic factor CHOP.

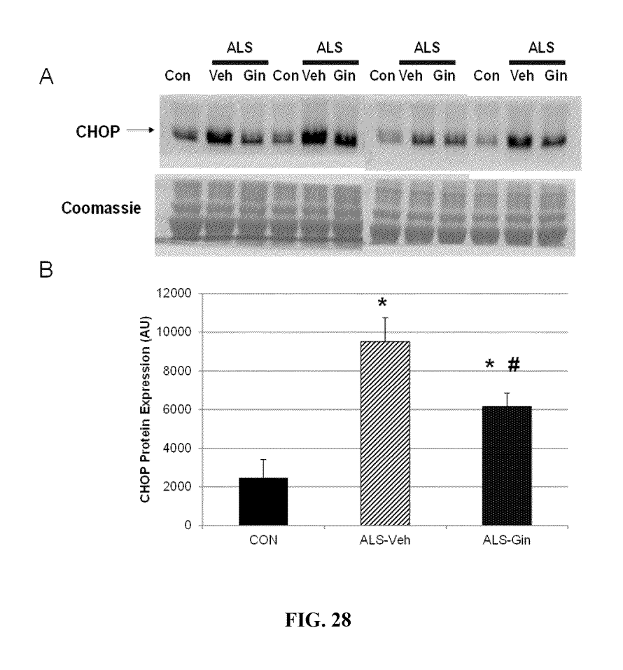

FIG. 28 showed that CHOP protein was upregulated in skeletal muscle of ALS-Veh compared to CON-Veh but was significantly attenuated in ALS-Gin mice. Coomassie blue staining of membrane is shown for a loading control.

These data showed that CHOP levels are reversed by administration of 6-gingerol to ALS mice. These data also showed that CHOP levels are significantly attenuated, which coincides with gains in muscle mass and function observed in ALS mice treated with 6-gingerol.

### Example 18

### Determination of Dose Response for 6-Gingerol in ALS Mice

Experimental Design.

Both wild-type control (CON) and G93A*SOD1 (ALS) mice are used in the study. Control and ALS mice are obtained from colonies established with male breeders of the B6SJL-Tg(SOD1-G93A)1Gur/J strain and wild-type C57BL/6xSJL females obtained from Jax laboratories. After weaning and genotyping mice at 21 d, mice are weighed weekly. Mice are randomized based on body weight at 35 d to one of the 5 treatment groups shown in Table 6.

TABLE 6
Genotype and drug treatment groups for dose-response study.

| | |
|---|---|
| CON | G93A*SOD1 |

| | | | |
|---|---|---|---|
| Veh | n = 48 | Veh | n = 48 |
| 6-gingerol 1 mg/kg | n = 48 | 6-gingerol 1 mg/kg | n = 48 |
| 6-gingerol 3 mg/kg | n = 48 | 6-gingerol 3 mg/kg | n = 48 |
| 6-gingerol 10 mg/kg | n = 48 | 6-gingerol 10 mg/kg | n = 48 |
| 6-gingerol 30 mg/kg | n = 48 | 6-gingerol 30 mg/kg | n = 48 |

Forty-eight mice (24 male and 24 female) are used in each group. In addition to 24 mice per group, the following are adhered to in the study: i) the use of litter matched control and treatment groups; ii) determination of gene copy number for all mice in therapeutic trials; and iii) censoring data of littermates from mice lost from the study due to non-ALS related events. Based on the data in Examples 11-14, a sample size (n) of 11 is required to detect an improvement in grip test, the primary outcome for assessing muscle function, and n=8 and n=10 to detect differences in stride length and Ca²⁺ decay time, two key secondary outcomes, at 80% power for detecting significance at the p<0.05 level. Drug treatment group sizes of 24 provide adequate power for our primary and secondary endpoints.

Drug Treatment.

6-gingerol is purchased from Carbosynth Limited, UK. The dose selection of 1, 3, 10 and 30 mg/kg is determined based on half-log increments above and below the study dose of 10 mg/kg in Examples 11-14. At 35 d, mice are assigned to treatment groups as indicated above and daily dosing is carried out from 35 d until mice show signs of paralysis (˜120-140 d). Mice are dosed by ip injection with vehicle (0.4% ethanol in PBS) or 6-gingerol (dissolved in ethanol and brought to volume in PBS). Volume dosed is adjusted weekly according to changes in body weight. Over this timeframe of dosing, mice progress from early symptom onset (75 d) to substantial distress (˜125 d) to paralysis (˜140 d). For mice exhibiting signs of muscle paralysis, death is scored as the inability of a mouse to right itself 30 s after being placed on its side. At the end of the lifespan of the mice (˜120-140 d), tissues are harvested either for immediate analysis (intracellular Ca²⁺ measurements in single fibres) or quick frozen in liquid nitrogen for subsequent analyses.

Endpoint Assessment.

Based on the primary focus of improving muscle function, the primary outcome in this study is grip test. Key secondary outcomes are stride length and Ca²⁺ decay time. Stride length provides an additional index of muscle function and Ca²⁺ decay time provides insight into a key mechanism (i.e., SERCA activity) by which the 6-gingerol drug is improves intracellular Ca²⁺ handling and thus, muscle health and function. Based on the data in Example 14, SERCA protein expression in muscle is also assessed as a biomarker of 6-gingerol activity. Skeletal muscle function is the primary outcome in these studies based on the overall strategy of trying to identify a therapeutic that improves muscle function, and translates to increased mobility and respiratory function which are clinical endpoints scored in the ALS Functional Rating Scale (ALS-FRS). Additional outcomes that are assessed are outlined in Table 7, along with a brief rationale for these assessments.

TABLE 7
Summary of endpoints assessed in 6-gingerol dose-response study

| Category | Endpoint | Rationale |
|---|---|---|
| ALS disease progression | Symptom onset | Assessment of overall animal health |
| | Survival | Assessment of life extension |
| Motor function | Grip test | Assessment of muscle strength |
| | Stride length | Assessment of animal mobility |
| | Rotarod running time | Early detectable change in motor |
| | | function (as early as 60 d; (35)) |
| Motoneuron integrity | Neuromuscular junction | Assessment of effects of 6-gingerol on |
| | innervation | maintenance of neuromuscular junction |
| Cellular mechanisms of | Single muscle fibre [Ca²⁺]ᵢ | Assessment of muscle Ca²⁺ handling |
| muscle contractile function | (resting; electrically- | properties |
| | evoked) | |
| | SERCA1/2 expression | |
| Skeletal muscle cellular | Caspase 3 and caspase 12 | Assessment of effects of 6-gingerol on |
| function | activation | possible mechanisms of action for |
| | Protein carbonyl content | improving skeletal muscle function |
| | | including an attenuation of apoptosis and |
| | | oxidative stress |

Motor Function Analysis via Grip Test.

Starting at 70 d, mice are assessed for motor function using a standard grip test. Briefly, mice are placed on a metal grid (20 cm wide, 40 cm long; grid placed 40 cm above table) and allowed to grip with fore- and hind-limb paws prior to inverting the grid. Timing begins once the grid is inverted and stops once the mouse can no longer hold the lid. The grip test is carried out once per week from 70-98 d and then twice a week from 99 d until termination of study.

Motor Function Analysis Via Stride Length Test.

Fore- and hind-limb paws are dipped in non-toxic paint and mice walk across a 120 cm table covered in white paper. A dark box with food is placed at the far end to encourage walking across the table. The distance between the fore- and hind-limb ink marks is then measured. Four strides per mouse are quantified and the average stride length determined

Motor Function Analysis Via Rotarod Running Time.

To assess motor co-ordination, mice run on the EzRod Rotarod apparatus (Accuscan Instruments) using a ramp increase protocol (0 to 60 rpm over 6 min) Mice are tested starting at 35 d. On the first day, mice are familiarized with the Rotarod and then tested on the second day. Thereafter mice are tested once per week from 35-98 d and twice per week from 99 d until study termination. Each test session involves 3 trials 20 min apart and are performed at approximately the same time of day. Latency to fall is recorded in seconds for each trial and the average of the 3 trials is used for analyses. This endpoint is a sensitive and early indicator of motor function in dystrophic mice.

Assessment of Motoneuron Integrity.

Skeletal muscle innervation is assessed using immunofluorescence staining of gastrocnemius muscle with α-Bungarotoxin and anti-neurofilament antibodies to stain motor endplates, and AcetylCholine Receptor (AChR) to stain motoneurons. Number of intact neuromuscular junction (NMJ) are quantified by counting the innervated fibres (end-plates that show overlap of neurofilament and AChR staining)

Analysis of Single Muscle Fibre Calcium Handling.

To assess intracellular Ca²⁺ handling in skeletal muscle, intact single muscle fibres are obtained from the FDB by collagenase. Fibres are loaded with Fura-2 AM, placed in a culture/stimulation chamber containing parallel electrodes on top of a Nikon TiU microscope and continuously perfused. Intracellular Ca²⁺ levels are assessed by the Fura-2 fluorescence ratio (ratio of excitation at 340 and 380 nm; emission at 510 nm) using the IonOptix Hyperswitch system. Resting Fura-2 as well as peak Fura-2 ratios in response to electrical stimulation are measured. Fibres are stimulated using 350 ms tetani, 0.5 ms pulse duration at 10, 30, 50, 70 and 100, 120 and 150 Hz stimulation frequencies with one min rest between frequencies. Peak Fura-2 at each frequency are determined by the average ratio in the last 100 ms of the 350 ms tetanus.

Assessment of SERCA activity is based on the Ca²⁺ decay time: the amount of time required for the Fura-2 peak to return back to 25% of baseline value at the end of a tetanus. This method assesses intracellular Ca²⁺ removal by the SR Ca²⁺ pump. Data from one CON and one ALS mouse (same drug treatment group) are collected on the same day to minimize variability between fibres. Due to the lengthy duration of this technique, data are collected on only 10 of the 24 mice per group and 10-12 fibres will be analyzed per mouse.

Measurement of SERCA1 and SERCA2 Protein Expression.

Skeletal muscles removed and quick frozen at the termination of each experiment are utilized to measure the level of SERCA1 and SERCA2 expression by western blot analyses.

Caspase 3 and Caspase 12 Activation.

To assess the level of apoptosis in skeletal muscle, western blot analyses of caspase 3 and caspase 12 are completed. Under conditions of apoptosis, caspase 3 and 12 are cleaved and a characteristic banding pattern is observed. Changes in both caspase 3 and caspase 12 cleavage in response to 6-gingerol treatment are also examined

Measurement of Protein Carbonyl Content.

To assess the level of total oxidative stress in skeletal muscle, protein carbonyl content is measured using the Protein Carbonyl Colorimetric Assay Kit (Cayman Chemical).

Pharmacokinetics (PK) and Pharmacodynamics (PD) Assessment.

To evaluate the PK/PD relationship of 6-gingerol, plasma is collected from a subset of 6 mice in each of the G93A*SOD1 6-gingerol drug treatment groups (all doses). Both plasma and muscle tissue content of 6-gingerol is measured by liquid chromatography tandem mass spectrometry (LC-MS/MS).

Data Analysis.

Data are analyzed using a two-way ANOVA for differences between genotype (CON vs. ALS) and between drug treatment groups. Tukey post hoc analyses is used for all significant ANOVAs and p<0.05 used to determine statistical significance. The dose-response relationship for key primary and secondary outcomes are analyzed by Hill-plot to determine the maximum effect and EC50 using SigmaStat software package. For PK/PD modeling, the SimBiology pharmacokinetics software is used.

### Example 19

### Effect of Increasing SERCA Protein Content in ALS Mice

Experimental Design.

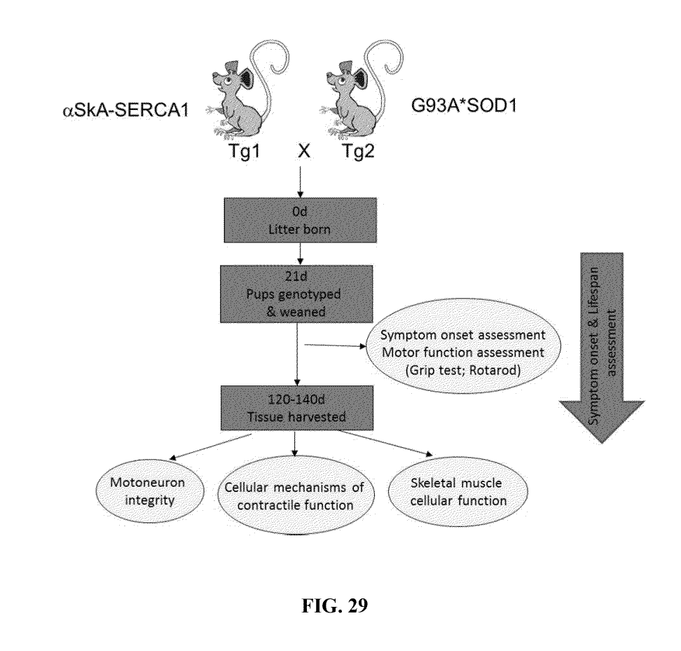

In order to evaluate the effect of increasing SERCA1 content in attenuating the pathological changes in skeletal muscle in ALS, the G93A*SOD1 mice is crossed or breed with the αSketelal Actinin (SkA)-SERCA1 Tg mice to obtain the double transgenic G93A*SOD1×SERCA1 mice. A flow chart to illustrate the study design for the transgenic mice is shown in FIG. 29. The genotype groups generated by the breeding scheme include: i) wild-type CON (25%); ii) G93A*SOD1 (25%); iii) SERCA1 (25%); and iv) G93A*SOD1×SERCA1 (25%). An n=24 mice (male and female, each) in each of the genotypes are assessed in the assays or endpoints described below.

Endpoint Assessment.

The endpoints assessed in this genetic study are the same as those assessed in the 6-gingerol dose response study outlined above and shown in Table 6.

An Alternative Approach to Non-Mendelian Ratios.

Experience with the G93A*SOD1 colony has shown that the number of transgenic mice expected by Mendelian ratios are generated. However, the number of αSkA-SERCA1 Tg mice per litter has been lower than expected. Thus a larger number of breeding cages are required to generate the required n=24 for G93A*SOD1×SERCA1 mice. Alternatively, an adenoviral construct can be used to overexpress SERCA1 (i.e., adeno-SERCA1 constructs) and injected into the G93A*SOD1 mice.

Analysis of Pharmacological Treatment vs. Genetic Study.

A comparison of outcomes from the pharmacological study with the small molecule SERCA activator (i.e., 6-gingerol) to those of the genetic study designed to increase SERCA1 protein level in skeletal muscle allows for an evaluation of the non-muscle specific and the off-target effects of 6-gingerol. Since the SERCA1 Tg mice only overexpress the SR Ca²⁺ pump in skeletal muscle, any efficacy observed with 6-gingerol on muscle function, motor co-ordination, or motoneuron integrity, but not in the G93A*SOD1×SERCA1 mice indicates either non-muscle (i.e. direct motoneuron) benefits of 6-gingerol treatment or ii) mechanisms of action of 6-gingerol beyond their effects on SR Ca²⁺ ATPase activity and SR Ca²⁺ clearance function. A role of 6-gingerol beyond SERCA activation is a modulation of the redox state of skeletal muscle or motoneurons based on the anti-oxidant and/or anti-inflammatory effects of 6-gingerol.

### Example 20

### Determination of SERCA Protein Content and Activity in Human ALS Subjects and Effects of 6-Gingerol in the Same

Experimental Design.

In view of the above data from the G93A*SOD1 mice, a decrease in SERCA protein level and SERCA Ca²⁺ pump function in skeletal muscle of ALS patients compared to healthy age-matched controls is examined. Muscle biopsies are obtained from ALS patients with known and unknown genetic mutations. The study evaluates human muscle biopsy samples from ALS patients with known SOD1 mutations (n=6), sporadic ALS patients (n=6), and healthy control subjects (n=6).

Endpoint Assessment.

The human skeletal muscle biopsy samples are assessed for SERCA content by western blot analysis and SERCA Ca²⁺ pump and ATPase activity is evaluated using human muscle homogenate assays. Methods for SERCA1/2 protein content are similar to that used for mouse SERCA1/2 content by western blot analyses as described in Example 1. Conditions are re-optimized for human muscle samples.

The SR Ca²⁺ ATPase and Ca²⁺ uptake assays are used to analyze human muscle homogenate samples. This study also evaluates the potential efficacy and the dose-responsiveness of 6-gingerol in increasing SR Ca²⁺ ATPase activity and Ca²⁺ uptake in human muscles in these in vitro assay systems. These in vitro SERCA assays provide an evaluation of 6-gingerol or other SERCA modulators as a novel therapeutic strategy for ALS.

### Example 21

### PERK, GRP78/BiP, PDI, and IRE1α Levels are Decreased in ALS Mice Administered 6-Gingerol

As described above in Examples 15-18, ALS mice administered 6-gingerol had improved muscle function, improved intracellular calcium clearance, increased SERCA1 protein levels, and decreased CHOP protein levels. To further examine the response of skeletal muscle to 6-gingerol treatment, the levels of ER stress proteins PERK, GRP78/BiP, PDI, and IRE1α were measured in the gastrocnemius muscle of control (CON) and G93A*SOD1 (ALS) mice treated with vehicle (Veh) or 6-gingerol (Gin). Specifically, the protein levels were measured by western blotting. Each group had five mice, i.e., CON (n=5), ALS-Veh (n=5), and ALS-Gin (n=5).

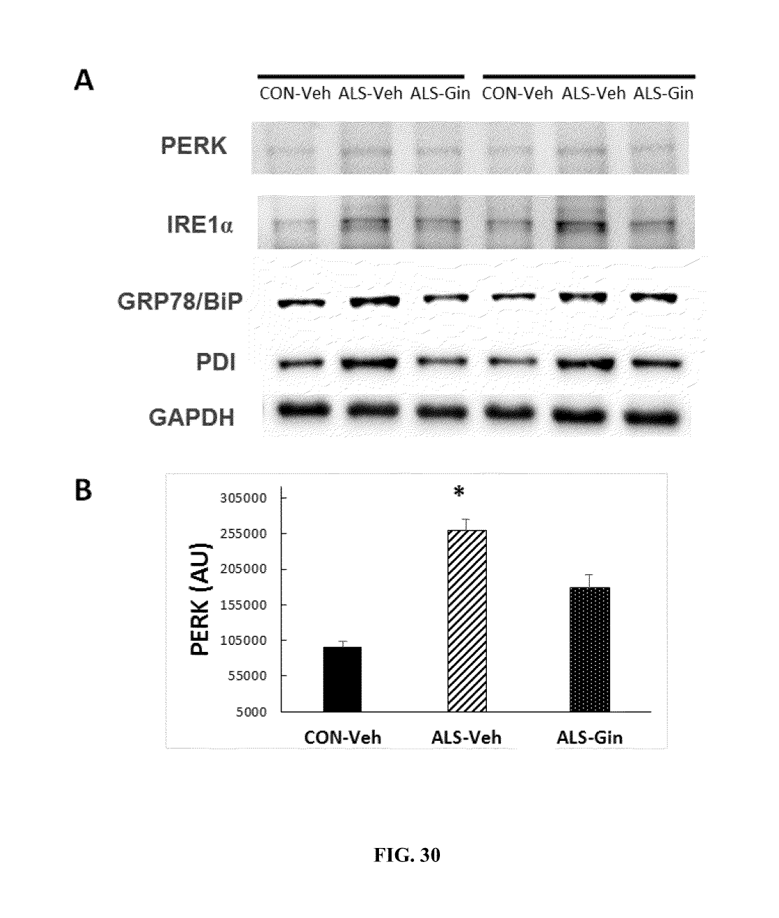

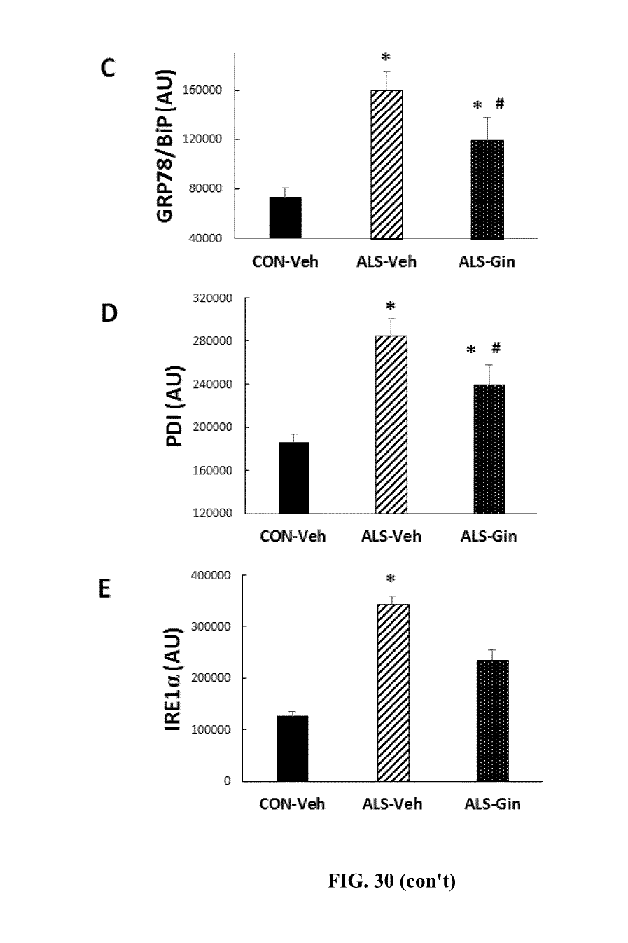

FIG. 30A depicts the western blot, which contained two representative data sets for each of PERK, GRP78/BiP, PDI, and IRE1α protein levels in the three groups of mice. GAPDH protein was used as a loading control. FIGS. 30B-32E depict the average data for each group of mice. In FIGS. 30B-30D, *=p<0.05 vs. CON-Veh and #=p<0.05 vs. ALS-Veh.

These results showed that PERK, GRP78/BiP, PDI, and IRE1α protein levels were significantly increased in the ALS mice (i.e., ALS-Veh group in FIGS. 30B-30D). These results also showed that PERK, GRP78/BiP, PDI, and IRE1α protein levels were decreased in ALS mice receiving the 6-gingerol treatment (i.e., ALS-Gin group) as compared to ALS mice that received the vehicle (i.e., ALS-Veh). As described above, ALS mice receiving 6-gingerol had improved grip function and stride function as well as improved muscle mass and intracellular calcium clearance. Accordingly, the protein levels of PERK, GRP78/BiP, PDI, and IRE 1α respond to ALS, i.e., go up in ALS disease and down in response to treatment of ALS.

### Example 22

### Maximum Ca²⁺ ATPase Activity is Increased in ALS Mice Administered 6-Gingerol

To further examine the effects of 6-gingerol treatment, maximum Ca²⁺ ATPase activity was measured in control (CON) and G93A*SOD1 (ALS) mice treated with vehicle (Veh) or 6-gingerol (Gingerol). Specifically, ALS mice received 10 mg/kg 6-gingerol for 10 weeks. The vehicle was administered in parallel to the CON-Veh and ALS-Veh groups of mice.

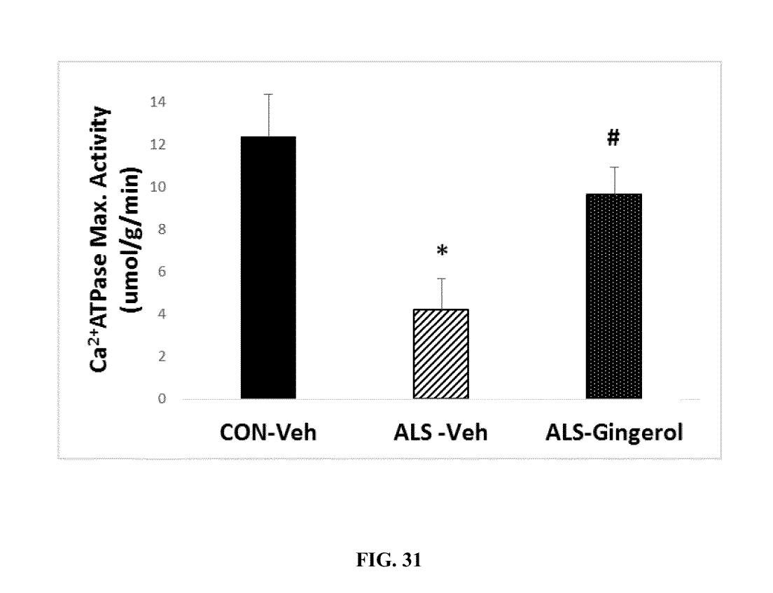

SR Ca²⁺ ATPase (SERCA) activity in skeletal muscle homogenates obtained from the mice was then measured and the average data for all groups is shown in FIG. 31, in which CON (n=5), ALS-Veh (n=5), and ALS-Gin (n=5). In FIG. 31, *=p<0.05 vs. CON-Veh and #=p<0.05 vs. ALS-Veh. These data demonstrated that 6-gingerol treatment of ALS mice resulted in increased SERCA activity relative to ALS mice that received the vehicle. Additionally, the 6-gingerol treatment brought SERCA activity back to the levels found in the CON mice, and thus, restored Ca²⁺ ATPase maximum activity.

### Example 23

### B-Actin Aggregates and α-Tubulin Solubility in the Skeletal Muscle of ALS Mice

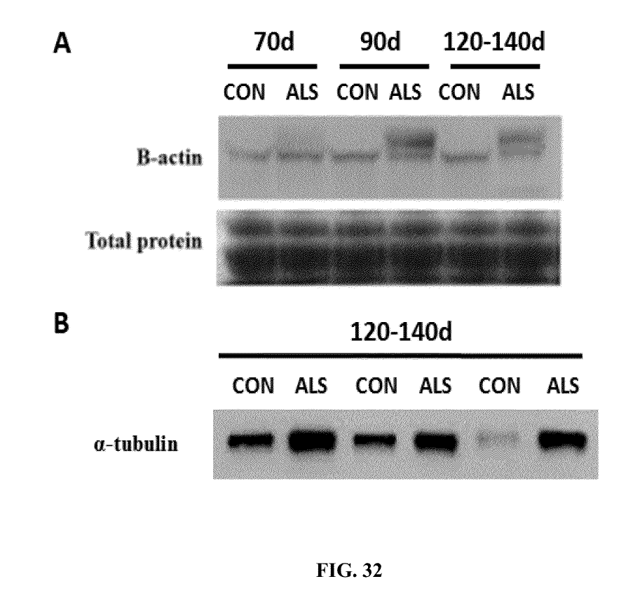

The proteins β-actin and α-tubulin were examined over time in the skeletal muscle of wild-type (CON) and G93A*SOD1 (ALS) mice. In particular, protein was isolated from white gastrocnemius muscle of CON and ALS mice at different ages (i.e., 70 days (d), 90 days, and 120-140 days). Total protein was obtained using the reversible protein stain kit (MEMCODE, Thermo Scientific) and used as a loading control. Western blotting was employed in combination with antibodies specific for α-tubulin or β-actin to detect these proteins. The blots and loading control are shown in FIGS. 32A and 32B.

For α-tubulin, solubility decreased with age as indicated by increased amounts of α-tubulin aggregates in the older mice (i.e., 120 d-140 d; FIG. 32B). The severity of ALS increased with age in the ALS mice. Accordingly, α-tubulin solubility decreased as the severity of ALS increased and thus, α-tubulin aggregates increased as ALS became more severe.

For β-actin, a doublet appeared in the 90 d age ALS mice, but was not observed in the 90 d age CON mice (FIG. 32A). This β-actin doublet was also observed in the 120 d-140 d age ALS mice and not in the 120 d-140 d age CON mice. This doublet represented the formation of aggregates of β-actin, which form in the presence of misfolded β-actin protein. Accordingly, these data demonstrated that as the severity of ALS increased (i.e., disease severity increased with age in ALS mice), β-actin protein became misfolded and formed β-actin aggregates. Thus, the formation and detection of β-actin aggregates tracked with the severity of ALS.

FIG. 35 summarizes the effects of ALS and 6-gingerol treatment on the proteins examined in the present and above examples. The white arrows in each box indicated the effect of 6-gingerol treatment while the solid black arrow in each box indicated the effect of ALS. As shown and discussed above, 6-gingerol treatment counter-acted the effects of ALS with regards to SR Ca²⁺ ATPase activity, Ca²⁺-handling proteins (e.g., SERCAs, PV), [Ca²⁺]ᵢ, levels, protein aggregates (e.g., β-actin and α-tubulin), protein synthesis (e.g., Akt, pAkt, P70S6K, pP70S6K), and the ER stress/unfolded protein response (e.g., PERK, IRE1α, GRP78/BiP, and CHOP).

### Example 24

### Detection of ALS in Humans

As described in the examples above, several proteins were altered (e.g., level, aggregate formation, and solubility) in the skeletal muscle of ALS mice as compared to wild-type mice. Accordingly, these proteins were examined in human skeletal muscle. Specifically, the levels of the following proteins were examined in human skeletal muscle from disease control and SOD1 AV4 sub-type of ALS: SERCA1, SERCA2, Akt, PDI, CHOP, β-actin, and α-tubulin. Protein expression was measured using primary antibodies against each respective protein, horse radish peroxidase (HRP)-linked secondary antibody, chemiluminescence, and quantification by densitometry.

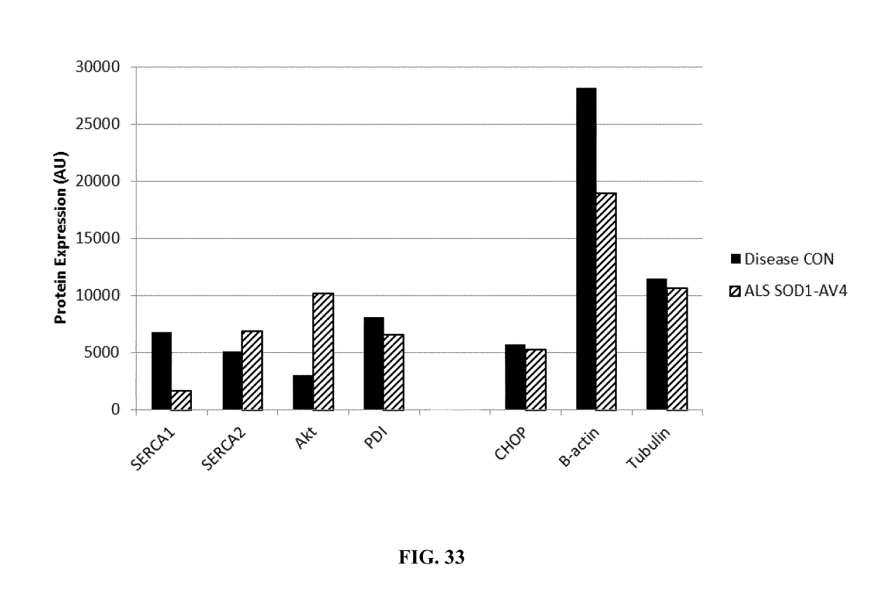

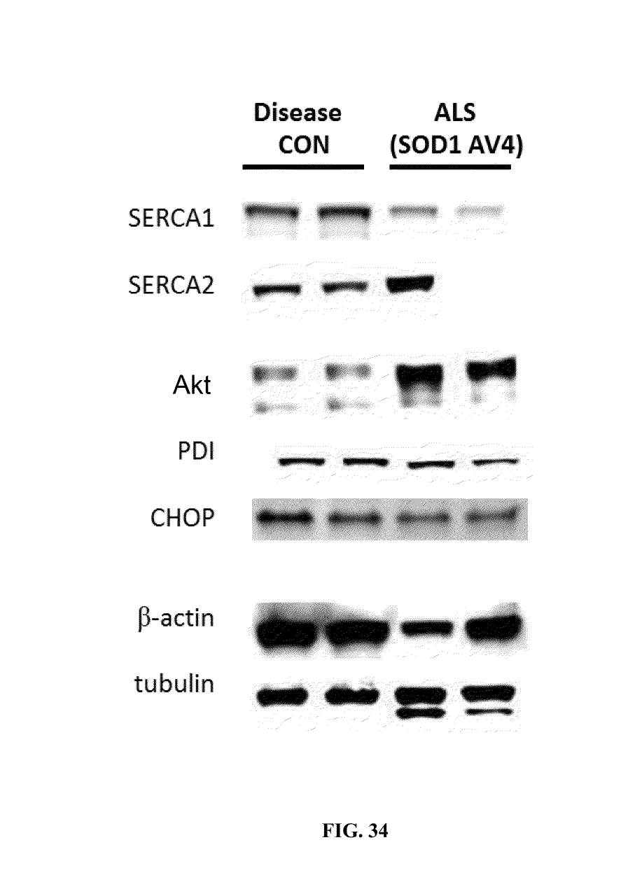

The results of this analysis are shown in FIGS. 33 and 34. Specifically, FIG. 34 shows the western blot images while FIG. 33 shows the average data for disease control (n=2) and SOD1 AV4 subtype of ALS (n=2). These data demonstrated decreased SERCA1 protein levels in the SOD1 AV4 subtype of ALS as compared to the disease control. These data also showed increased Akt protein levels in the SOD1 AV4 subtype of ALS as compared to the disease control. These data further showed alterations in the aggregation of β-actin and solubility of α-tubulin in the SOD1 AV4 subtype of ALS as compared to the disease control. Accordingly, decreased SERCA1 protein levels, increased Akt protein levels, and alterations in β-actin aggregates and α-tubulin solubility coincided with the presence of ALS in humans.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the present disclosure are set out in the following numbered clauses:

Clause 1. A method for diagnosing amyotrophic lateral sclerosis (ALS) in a subject in need thereof, the method comprising: (a) obtaining a sample from the subject; (b) measuring levels of sarcoplasmic reticulum endoplasmic reticulum 1 (SERCA 1) and SERCA 2 proteins in the sample; and (c) comparing the levels measured in step (b) with levels of SERCA 1 and SERCA 2 proteins in a control, wherein a decrease in the levels of SERCA 1 and SERCA 2 proteins as compared to the control indicate that the subject is suffering from ALS.

Clause 2. The method of claim 1, wherein the sample includes at least one of a plasma sample, a serum sample, and a skeletal muscle tissue sample.

Clause 3. The method of claim 1, further comprising measuring a Ca²⁺ level in the sample, and comparing the Ca²⁺ level to a Ca²⁺ level in the control, wherein an increase in the Ca²⁺ level as compared to the control further indicates that the subject is suffering from ALS.

Clause 4. The method of claim 3, wherein the Ca²⁺ level is an intracellular Ca²⁺ concentration.

Clause 5. The method of claim 3, further comprising measuring a level of parvalbumin (PV) protein in the sample, and comparing the level of PV protein to a level of PV protein in the control, wherein a decrease in the level of PV protein as compared to the control further indicates that the subject is suffering from ALS.

Clause 6. The method of claim 1, further comprising measuring a level of an mRNA selected from a group consisting of SERCA 2 mRNA, TnIs mRNA, and Myoglobin mRNA, and comparing the measured level to a level of a corresponding mRNA in the control, wherein an increase in the level of SERCA 2, TnIs, or Myoglobin mRNA as compared to the control further indicates that the subject is suffering from ALS.

Clause 7. The method of claim 1, further comprising measuring a level of an mRNA selected from a group consisting of TnIf mRNA, GAPDH mRNA, and MCK mRNA, and comparing the measured level to a level of a corresponding mRNA in the control, wherein a decrease in the level of TnIf, GAPDH, or MCK mRNA as compared to the control further indicates that the subject is suffering from ALS.

Clause 8. The method of claim 1, further comprising measuring a level of endoplasmic reticulum (ER) chaperone immunoglobin binding protein (BiP), and comparing the measured level to a level of BiP protein in the control, wherein an increase in the level of BiP protein as compared to the control further indicates that the subject is suffering from ALS.

Clause 9. The method of claim 8, further comprising measuring a level of a protein selected from a group consisting of PERK, IRE1α, PDI, CHOP, and Caspase-12, and comparing the measured level of the protein to a level of a corresponding protein in the control, wherein an increase in the level of PERK, IRE1α, PDI, CHOP, or Caspase-12 protein further indicates that the subject is suffering from ALS.

Clause 10. The method of claim 1, further comprising measuring a level of an aggregate selected from the group consisting of β-actin aggregate, α-tubulin aggregate, and a combination thereof, and comparing the measured level of the aggregate to a level of a corresponding aggregate in the control, wherein an increase in the level of β-actin aggregate or α-tubulin aggregate further indicates that the subject is suffering from ALS.

Clause 11. The method of claim 10, wherein the measured level of the aggregate indicates a severity of ALS.

Clause 12. A method for diagnosing amyotrophic lateral sclerosis (ALS) in a subject in need thereof, the method comprising: (a) obtaining a sample from a subject; (b) measuring a level of endoplasmic reticulum (ER) chaperone immunoglobin binding protein (BiP) in the sample; and (c) comparing the level measured in step (b) with a level of BiP protein in a control, wherein an increase in the level of BiP protein as compared to the control indicates that the subject is suffering from ALS.

Clause 13. The method of claim 12, wherein the sample includes at least one of a plasma sample, a serum sample, and a skeletal muscle sample.

Clause 14. The method of claim 12, further comprising measuring a level of a protein selected from a group consisting of PERK, IRE1α, and PDI, and comparing the measured level of the protein to a level of a corresponding protein in the control, wherein an increase in the level of PERK, IRE1α, or PDI protein further indicates that the subject is suffering from ALS.

Clause 15. The method of claim 12, further comprising measuring a level of a protein selected from a group consisting of CHOP and Caspase-12, and comparing the measured level of the protein to a level of a corresponding protein in the control, wherein an increase in the level of CHOP or Caspase-12 protein further indicates that the subject is suffering from ALS.

Clause 16. The method of claim 12, further comprising measuring a level of a protein selected from the group consisting of sarcoplasmic reticulum endoplasmic reticulum 1 (SERCA 1) and SERCA 2, and comparing the measured level of the protein to a level of the corresponding protein in the control, wherein a decrease in the level of SERCA1 or SERCA2 protein further indicates that the subject is suffering from ALS.

Clause 17. The method of claim 16, further comprising measuring a level of parvalbumin (PV) protein in the sample, and comparing the level of PV protein to a level of PV protein in the control, wherein a decrease in the level of PV protein as compared to the control further indicates that the subject is suffering from ALS.

Clause 18. The method of claim 16, further comprising measuring a level of an mRNA selected from a group consisting of SERCA 2 mRNA, TnIs mRNA, and Myoglobin mRNA, and comparing the measured level to a level of a corresponding mRNA in the control, wherein an increase in the level of SERCA 2, TnIs, or Myoglobin mRNA as compared to the control further indicates that the subject is suffering from ALS.

Clause 19. The method of claim 16, further comprising measuring a level of an mRNA selected from a group consisting of TnIf mRNA, GAPDH mRNA, and MCK mRNA, and comparing the measured level to a level of a corresponding mRNA in the control, wherein a decrease in the level of TnIf, GAPDH, or MCK mRNA as compared to the control further indicates that the subject is suffering from ALS.

Clause 20. The method of claim 12, further comprising measuring a level of an aggregate selected from the group consisting of β-actin aggregate, α-tubulin aggregate, and a combination thereof, and comparing the measured level of the aggregate to a level of a corresponding aggregate in the control, wherein an increase in the level of β-actin aggregate or α-tubulin aggregate further indicates that the subject is suffering from ALS.

Clause 21. The method of claim 20, wherein the measured level of the aggregate indicates a severity of ALS.

Clause 22. A kit for early diagnosis of amyotrophic lateral sclerosis (ALS) in a subject, the kit comprising agents that bind and identify SERCA 1, SERCA 2, BiP, or a combination thereof.

Clause 23. The kit of claim 22, wherein the agents include antibodies.

Clause 24. The kit of claim 22, further comprising agents that detect a change in an mRNA selected from a group consisting of SERCA 2 mRNA, TnIs mRNA, Myoglobin mRNA, TnIf mRNA, GAPDH mRNA, MCK mRNA, and any combination thereof.

Clause 25. The kit of claim 22, further comprising agents that detect a change in an intracellular Ca²⁺ concentration.

Clause 26. The kit of claim 22, further comprising agents that bind and identify PERK, IRE1α, PDI, CHOP, Caspase-12, β-actin, α-tubulin, or a combination thereof.

Clause 27. A method for monitoring the efficacy of a treatment for amyotrophic lateral sclerosis (ALS) in a subject, the method comprising: (a) obtaining a first sample from the subject before the treatment and a second sample from the subject during or after treatment; (b) measuring a first level of a protein in the first sample and a second level of the protein in the second sample, wherein (i) the protein is selected from the group consisting of SERCA1 and PV; or (ii) the protein is selected from the group consisting of CHOP, Caspase-12, PERK, IRE1α, BiP, and PDI; and (c) comparing the first level of the protein and the second level of the protein, wherein (i) a second level of the protein during or after treatment of (b)(i) is higher than the first level of the protein of (b)(i) before treatment and is indicative of a therapeutic effect of the treatment in the subject; or (ii) a second level of the protein during or after treatment of (b)(ii) is lower than the first level of the protein of (b)(ii) before treatment and is indicative of a therapeutic effect of the treatment in the subject.

Clause 28. The method of claim 27, wherein the protein of (b)(i) is SERCA1.

Clause 29. The method of claim 27, wherein the protein of (b)(ii) is CHOP, PERK, IRE1α, BiP or PDI.

Clause 30. A method for treatment of amyotrophic lateral sclerosis (ALS) in a subject in need thereof, the method comprising administering a composition comprising a therapeutically effective amount of an agent, wherein the agent is 6-gingerol.

Clause 31. A method for treatment of amyotrophic lateral sclerosis (ALS) in a subject in need thereof, the method comprising administering a composition comprising a therapeutically effective amount of an agent that increases a level of SERCA1 protein.

Clause 32. The method of claim 25, wherein the agent decreases a level of CHOP protein, PERK protein, IRE1α protein, BiP protein, PDI protein, or any combination thereof

What is claimed is:

1. A method for diagnosing amyotrophic lateral sclerosis (ALS) in a subject in need thereof, the method comprising:
(a) obtaining a sample from the subject;
(b) measuring levels of sarcoplasmic reticulum endoplasmic reticulum 1 (SERCA 1) and SERCA 2 proteins in the sample; and
(c) comparing the levels measured in step (b) with levels of SERCA 1 and SERCA 2 proteins in a control,
wherein a decrease in the levels of SERCA 1 and SERCA 2 proteins as compared to the control indicate that the subject is suffering from ALS.

2. The method of claim 1, wherein the sample includes at least one of a plasma sample, a serum sample, and a skeletal muscle tissue sample.

3. The method of claim 1, further comprising measuring a Ca²⁺ level in the sample, and comparing the Ca²⁺ level to a Ca²⁺ level in the control, wherein an increase in the Ca²⁺ level as compared to the control further indicates that the subject is suffering from ALS.

4. The method of claim 3, wherein the Ca²⁺ level is an intracellular Ca²⁺ concentration.

5. The method of claim 3, further comprising measuring a level of parvalbumin (PV) protein in the sample, and comparing the level of PV protein to a level of PV protein in the control, wherein a decrease in the level of PV protein as compared to the control further indicates that the subject is suffering from ALS.

6. The method of claim 1, further comprising measuring a level of an mRNA selected from a group consisting of SERCA 2 mRNA, TnIs mRNA, and Myoglobin mRNA, and comparing the measured level to a level of a corresponding mRNA in the control, wherein an increase in the level of SERCA 2, TnIs, or Myoglobin mRNA as compared to the control further indicates that the subject is suffering from ALS.

7. The method of claim 1, further comprising measuring a level of an mRNA selected from a group consisting of TnIf mRNA, GAPDH mRNA, and MCK mRNA, and comparing the measured level to a level of a corresponding mRNA in the control, wherein a decrease in the level of TnIf, GAPDH, or MCK mRNA as compared to the control further indicates that the subject is suffering from ALS.

8. The method of claim 1, further comprising measuring a level of endoplasmic reticulum (ER) chaperone immunoglobin binding protein (BiP), and comparing the measured level to a level of BiP protein in the control, wherein an increase in the level of BiP protein as compared to the control further indicates that the subject is suffering from ALS.

9. The method of claim 8, further comprising measuring a level of a protein selected from a group consisting of PERK, IRE1α, PDI, CHOP, and Caspase-12, and comparing the measured level of the protein to a level of a corresponding protein in the control, wherein an increase in the level of PERK, IRE1α, PDI, CHOP, or Caspase-12 protein further indicates that the subject is suffering from ALS.

10. The method of claim 1, further comprising measuring a level of an aggregate selected from the group consisting of β-actin aggregate, α-tubulin aggregate, and a combination thereof, and comparing the measured level of the aggregate to a level of a corresponding aggregate in the control, wherein an increase in the level of β-actin aggregate or α-tubulin aggregate further indicates that the subject is suffering from ALS.

11. The method of claim 10, wherein the measured level of the aggregate indicates a severity of ALS.

12. A method for diagnosing amyotrophic lateral sclerosis (ALS) in a subject in need thereof, the method comprising:
(a) obtaining a sample from a subject;
(b) measuring a level of endoplasmic reticulum (ER) chaperone immunoglobin binding protein (BiP) in the sample; and
(c) comparing the level measured in step (b) with a level of BiP protein in a control,
wherein an increase in the level of BiP protein as compared to the control indicates that the subject is suffering from ALS.

13. The method of claim 12, wherein the sample includes at least one of a plasma sample, a serum sample, and a skeletal muscle sample.

14. The method of claim 12, further comprising measuring a level of a protein selected from a group consisting of PERK, IRE1α, and PDI, and comparing the measured level of the protein to a level of a corresponding protein in the control, wherein an increase in the level of PERK, IRE1α, or PDI protein further indicates that the subject is suffering from ALS.

15. The method of claim 12, further comprising measuring a level of a protein selected from a group consisting of CHOP and Caspase-12, and comparing the measured level of the protein to a level of a corresponding protein in the control, wherein an increase in the level of CHOP or Caspase-12 protein further indicates that the subject is suffering from ALS.

16. The method of claim 12, further comprising measuring a level of a protein selected from the group consisting of sarcoplasmic reticulum endoplasmic reticulum 1 (SERCA 1) and SERCA 2, and comparing the measured level of the protein to a level of the corresponding protein in the control, wherein a decrease in the level of SERCA1 or SERCA2 protein further indicates that the subject is suffering from ALS.

17. The method of claim 16, further comprising measuring a level of parvalbumin (PV) protein in the sample, and comparing the level of PV protein to a level of PV protein in the control, wherein a decrease in the level of PV protein as compared to the control further indicates that the subject is suffering from ALS.

18. The method of claim 16, further comprising measuring a level of an mRNA selected from a group consisting of SERCA 2 mRNA, TnIs mRNA, and Myoglobin mRNA, and comparing the measured level to a level of a corresponding mRNA in the control, wherein an increase in the level of SERCA 2, TnIs, or Myoglobin mRNA as compared to the control further indicates that the subject is suffering from ALS.

19. The method of claim 16, further comprising measuring a level of an mRNA selected from a group consisting of TnIf mRNA, GAPDH mRNA, and MCK mRNA, and comparing the measured level to a level of a corresponding mRNA in the control, wherein a decrease in the level of TnIf, GAPDH, or MCK mRNA as compared to the control further indicates that the subject is suffering from ALS.

20. The method of claim 12, further comprising measuring a level of an aggregate selected from the group consisting of β-actin aggregate, α-tubulin aggregate, and a combination thereof, and comparing the measured level of the aggregate to a level of a corresponding aggregate in the control, wherein an increase in the level of β-actin aggregate or α-tubulin aggregate further indicates that the subject is suffering from ALS.

21. The method of claim 20, wherein the measured level of the aggregate indicates a severity of ALS.


SEQUENCE LISTING (2 sequences)

SEQ ID NO: 1 - DNA, 22 bp, Artificial Sequence
  Synthetic polynucleotide
tgcctggtggagaagatgaatg

SEQ ID NO: 2 - DNA, 22 bp, Artificial Sequence
  Synthetic polynucleotide
ctgtttgacaccaggagtcatg